(19) United States
(12) Patent Application Publication
(10) Pub. No.: US 2026/0038673 A1
(43) Pub. Date: Feb. 5, 2026

(54) COMPUTER SYSTEMS FOR CHECK POINT IDENTIFICATION

(71) Applicant: Tempus AI, Inc., Chicago, IL (US)

(72) Inventors: Logan Xavier Brigman, Milwaukee, WI (US); Cara Marie Feind, Hartford, WI (US); Chris Michael Rogers, Oconomowoc, WI (US); Noah Zimmerman, Redwood City, CA (US)

(21) Appl. No.: 18/789,091

(22) Filed: Jul. 30, 2024

Related U.S. Application Data
(60) Provisional application No. 63/676,986, filed on Jul. 30, 2024.

(51) Int. Cl. G16H 40/20; G06N 5/01

(52) U.S. Cl. CPC G16H 40/20; G06N 5/01

(57) ABSTRACT

Computer systems for check point identification for a subject through in silico modeling are provided. One or more conditions and a plurality of contacts associated with the subject are identified in a data repository. A subset of decision rules is discovered from among a plurality of decision rules through alignment of a condition in the one or more conditions against the decision rules. The subset of decision rules models a response to the first condition at a first entity. A first decision rule in the subset of decision rules is activated using an evaluation module when a corresponding triggering condition for the decision rule arises in the data repository. A notification rule actionable upon the activating of the first decision rule is identified. A notification is communicated using a computer network to a notification path, consisting of a subset of the plurality of contacts, in accordance with the notification rule.

### RELATED APPLICATIONS

This application claims priority to U.S. Provisional Application No. 63/676,986, filed on Jul. 30, 2024, and entitled “COMPUTER SYSTEMS FOR CHECK POINT IDENTIFICATION,” which is hereby incorporated by reference in its entirety.

### TECHNICAL FIELD

Disclosed are technologies generally relating to check point identification.

### BACKGROUND

With the advent of increasingly sophisticated medical care, such as increasingly detailed clinical guidelines, biomarker informed personalized medicine, increasingly complex medical devices, and increasingly detailed medical procedures such as imaging techniques, there is a need in the art to ensure that subjects are receiving suitable medical care. For many medical treatments, there is an applicable standard of care promulgated by the medical community. However, determining whether such standards of care are being satisfied at all times is difficult to determine.

First, determination of what standard of care a subject is receiving requires evaluation of the medical data associated with the subject's medical treatment. But such medical data comes in numerous different forms and sources (modalities) such as, to name a few sources, electronic health records, medical test results, physician notes, type of equipment used, patient questionnaire results, etc. Beyond the fact that there are multiple sources for such data, the data itself does not adhere to a common form. For instance, while electronic health records often have a structured form, physician notes are free form and do not.

Second, applicable standards of care are embodied in many different ways, and are updated frequently. For instance, with the advent of biomarker informed personalized medical care, applicable standards of care have many different branch points each informed by subject specific conditions, such as past treatment, age, ethnicity and other covariates, biomarkers, etc. Further still, applicable standards of care often include the type of medical equipment used, the version of software used in such medical equipment, and the certifications of the medical practitioners using such equipment. Further still, applicable standards of care are often not static, and include a temporal component as well. Examples of this temporal component include whether follow-up medical consultations after a medical procedure have been made, whether laboratory procedures to measure diagnostic indicators of a disease condition have been made on an ongoing basis, etc. One specific example of this temporal component is checking to make sure a subject that medical guidelines indicate should have a colonoscopy every five years, has in fact, gotten one in the past five years. Another specific example of this temporal component is checking to make sure a subject that has a pacemaker is having the pacemaker checked by medical practitioners on at least an annual basis.

A check point can arise when the available medical data for a subject indicates that some aspect of the subject's medical care is not compliant with an applicable standard of care. A check point can also arise when outdated equipment or processes are being used in imaging or diagnostics are detected, scheduling and follow-up need to be verified, applicable standards of care indicate that medical procedure to identify new tumors or nodules in the subject are warranted, etc.

A check point can also arise when the available medical data for a subject indicates that the subject may be eligible for a particular treatment that the subject is not currently receiving. This particular treatment is not necessarily part of an applicable standard of care. For example, a subject, based on their unique characteristics, such as age, sex, location, stage of disease, biomarker results, etc., may qualify for a particular clinical trial. In such an instance, a check point can arise when the subject is eligible for this clinical trial but is not currently enrolled in the clinical trial, even though the clinical trial is not part of an applicable standard of care.

Given the above background, systems and methods for improved check point identification are needed in the art.

### SUMMARY

The present disclosure addresses the above-identified shortcomings by providing computer systems for check point identification.

A computer system for check point identification for a subject through in silico modeling is provided. The computer system comprises a memory and a processor. The memory stores a plurality of processor executable instructions executable by the processor.

The plurality of processor executable instructions comprises instructions for identifying one or more first conditions associated with a subject, and a plurality of contacts associated with the subject, in a data repository comprising a plurality of treatment information units associated with the subject.

In some embodiments, the plurality of processor executable instructions further comprises instructions for updating the data repository using multimodal treatment data for the subject.

In some embodiments, the one or more first conditions comprises a first diagnosis associated with the subject.

In some embodiments, the plurality of contacts associated with the subject comprises a third party sponsoring a clinical trial.

In some embodiments, the plurality of contacts associated with the subject includes the subject.

In some embodiments, the plurality of contacts associated with the subject includes a plurality of caregivers that treat the subject at a first institution.

The plurality of processor executable instructions further comprises instructions for discovering a first subset of decision rules from among a plurality of decision rules through a modeling procedure that aligns a first condition in the one or more first conditions against the plurality of decision rules. Each decision rule in the first subset of decision rules has a corresponding triggering condition. The first subset of decision rules models a response to the first condition at a first entity.

In some embodiments, the plurality of decision rules includes a plurality of subsets of decision rules. Each respective subset of decision rules in the plurality of subsets of decision rules models a respective institution based care plan for a diagnosis in a plurality of diagnoses.

In some embodiments, the plurality of decision rules model respective institution based care plans for more than 50 diagnoses.

In some embodiments, the plurality of decision rules is in the form of a directed graph comprising a plurality of nodes and a plurality of edges. Each respective node in the plurality of nodes is a decision rule in the plurality of decision rules. Each edge in the plurality of edges links a respective first node in the plurality of nodes with at least one other respective second node in the plurality of nodes and specifies what conditions are to be satisfied by the data in the data repository in order to include the decision rule of the at least one other respective second node in the first subset of decision rules when the decision rule of the first respective node is included in the first subset of decision rules. The instructions for discovering the first subset of decision rules from among the plurality of decision rules through a modeling procedure that aligns a first condition in the one or more first conditions against the plurality of decision rules comprises traversing the directed graph beginning at an initial node in the directed graph.

In some embodiments, the plurality of decisions rules is in the form of a decision tree comprising a plurality of decisions. Each respective decision in the plurality of decisions dictates whether or not to include a decision rule in the plurality of decision rules in the first subset of decision rules based on satisfying or failing to satisfy one or more conditions using data in the data repository. The instruction for discovering the first subset of decision rules from among the plurality of decision rules through a modeling procedure that aligns a first condition in the one or more first conditions against the plurality of decision rules comprises traversing the decision tree beginning at an initial decision in the decision tree.

In some embodiments, the plurality of processor executable instructions further comprises instructions for activating, using an evaluation module, a first decision rule in the first subset of decision rules whose corresponding triggering condition arises in the data repository.

In some embodiments, the corresponding triggering condition for the first decision rule arises in the data repository when the data repository is missing a diagnostic test result specified by the first decision rule.

In some embodiments, the corresponding triggering condition for the first decision rule arises in the data repository when the data repository includes a diagnostic test result that satisfies a diagnostic test result threshold.

In some embodiments, the plurality of instructions further comprises instructions for updating the data repository using multimodal treatment data for the subject, and the corresponding triggering condition for the first decision rule comprises an absence of an evaluation of the subject in the multimodal treatment data for the subject during a predetermined time period associated with the first decision rule.

In some embodiments, the corresponding triggering condition for the first decision rule comprises an indication in the data repository that the subject has been prescribed a combination of medications having a documented adverse pharmacodynamic or pharmacokinetic interaction with each other.

In some embodiments, the corresponding triggering condition for the first decision rule comprises an absence of an indication in the data repository of a clinical trial for which the subject is currently eligible.

In some embodiments, the corresponding triggering condition for the first decision rule comprises an absence of a therapy in the data repository.

In some embodiments, the corresponding triggering condition for the first decision rule arises in the data repository when the data repository is missing a medical procedure.

In some embodiments, the corresponding triggering condition for the first decision rule arises in the data repository when the data repository includes a medical procedure result that specifies the subject has a predetermined condition specified by the corresponding triggering condition of the first decision rule.

In some embodiments, the response to the first condition at the first entity is a first institution based care plan at an institution and the first subset of decision rules model the first institution based care plan for a first diagnosis indicated by the activating of the first decision rule as a first decision tree.

The plurality of processor executable instructions further comprises instructions for identifying a first notification rule in a notification rule repository that is actionable upon the activating of the first decision rule.

The plurality of processor executable instructions further comprises instructions for communicating a first notification to a first notification path in accordance with the first notification rule, using a computer network. The first notification path consists of a first subset of the plurality of contacts.

In some embodiments, the response to the first condition at the first entity is a first institution based care plan at an institution and the first notification path is an identity of a first contact in the plurality of contacts having a first specialty associated with activation of the first decision rule.

In some embodiments, a second notification is communicated to a second contact having the first specialty when the first contact fails to respond to the first notification.

In some embodiments, the response to the first condition at the first entity is a first institution based care plan at an institution and the first notification path is an identity of a primary caregiver for the subject, in the plurality of contacts associated with the subject, at the institution.

In some embodiments, the response to the first condition at the first entity is a first institution based care plan at an institution and the first notification path is an identity of a caregiver for the subject, in the plurality of contacts associated with the subject, that most recently attended to the subject at the institution.

In some embodiments, the response to the first condition at the first entity is a first institution based care plan at an institution and the first notification path is an identity of a caregiver for the subject, in the plurality of contacts associated with the subject, that most frequently attends to the subject at the institution.

In some embodiments, the response to the first condition at the first entity is a first institution based care plan at an institution and the first notification path is an identity of a caregiver for the subject, in the plurality of contacts associated with the subject, that has been assigned responsibility for administering an upcoming medical procedure in accordance with the first institution based care plan.

In some embodiments, the first notification rule is associated with a first type of caregiver and, when the plurality of contacts associated with the subject includes a first caregiver of the first type, the first notification path includes the first caregiver of the first type, and when the plurality of contacts associated with the subject does not include a first caregiver of the first type, the first notification path includes a primary caregiver, in the plurality of contacts associated with the subject, associated with the subject.

In some embodiments, the plurality of processor executable instructions further comprises instructions for firing, using the evaluation module, a second decision rule in the first subset of decision rules whose corresponding triggering condition arises in the data repository and instructions for suppressing a notification associated with the second decision rule when the first decision rule is fired.

In some embodiments, the plurality of processor executable instructions further comprises instructions for firing, using the evaluation module, a second decision rule in the first subset of decision rules whose corresponding triggering condition arises in the data repository. A second notification rule in the notification rule repository is identified that is actionable upon the firing of the second decision rule. The second notification rule is a hierarchical notification rule that is dependent upon the firing of the first decision rule. A second notification is communicated to a second notification path specified by the second notification rule. The second notification path consists of a second subset of the plurality of contacts.

Still another aspect of the present disclosure provides a non-transitory computer-readable medium storing computer code comprising instructions, when executed by one or more processors, causing the processors to perform any of the methods described in the present disclosure.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an exemplary system for check point identification, in accordance with some embodiments of the present disclosure.

FIGS. 2A, 2B, 2C, 2D, 2E, 2F, and 2G provide an example flowchart depicting an example process for check point identification, in accordance with some embodiments of the present disclosure.

FIG. 3 illustrates a decision tree in accordance with some embodiments of the present disclosure.

FIG. 4 illustrates another decision tree in accordance with some embodiments of the present disclosure.

FIG. 5 illustrates a first notification in accordance with some embodiments of the present disclosure.

FIG. 6 illustrates logic used to decide whether or not certain notifications are communicated based on which decision rules have been activated in accordance with some embodiments of the present disclosure.

FIG. 7 illustrates multimodal data that is used in accordance with some embodiments of the present disclosure to discover care gaps in the form of triggering conditions.

FIGS. 8A, 8B, 8C, and 8D illustrate the discovery of a care gap identified for EGFR, ALK, and PDLI testing for a patient that is eligible and should have received it based on clinical guidelines, in accordance with an embodiment of the present disclosure.

FIG. 9 illustrates subsequent testing for biomarkers upon completion of the flowchart of FIG. 8, in accordance with an embodiment of the present disclosure.

FIGS. 10A, 10B, and 10C illustrate the discovery of a care gap in a non-small cell lung cancer patient and action taken in accordance with an embodiment of the present disclosure.

FIG. 11 illustrates how care gaps in diseases can be tracked across an institution in accordance with an embodiment of the present disclosure.

FIGS. 12A and 12B illustrate a report in accordance with an embodiment of the present disclosure.

### DETAILED DESCRIPTION

The present disclosure addresses the challenges in the art described in the above background by providing computer systems for check point identification for a subject through in silico modeling. One or more conditions and a plurality of contacts associated with the subject are identified in a data repository. A subset of decision rules is discovered from among a plurality of decision rules through alignment of a condition in the one or more conditions against the decision rules. The subset of decision rules models a response to the first condition at a first entity, such as a health care institutions. A first decision rule in the subset of decision rules is activated using an evaluation module when a corresponding triggering condition for the decision rule arises in the data repository. A notification rule actionable upon the activating of the first decision rule is identified. A notification is communicated using a computer network to a notification path, consisting of a subset of the plurality of contacts, in accordance with the notification rule. In so doing, the present disclosure helps to identify and contextualize subjects (e.g., patients) in their care journey, surface precession care pathways at point of care, track subjects for timely follow-up to help improve outcomes, monitor notification effectiveness and guidelines adoption, and support increase equitable access to precision care.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Plural instances may be provided for components, operations or structures described herein as a single instance. Finally, boundaries between various components, operations, and data stores are somewhat arbitrary, and particular operations are illustrated in the context of specific illustrative configurations. Other forms of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first dataset could be termed a second dataset, and, similarly, a second dataset could be termed a first dataset, without departing from the scope of the present invention. The first dataset and the second dataset are both datasets, but they are not the same dataset.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

Furthermore, when a reference number is given an “i^{th}” denotation, the reference number refers to a generic component, set, or embodiment. For instance, a cellular-component termed “cellular-component i” refers to the i^{th} cellular-component in a plurality of cellular-components.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Definitions

Unless defined otherwise, all technical and scientific terms used herein have the meaning commonly understood by one of ordinary skill in the art to which the invention pertains.

As used herein, the terms “abundance,” “abundance level,” or “expression level” refers to an amount of a cellular constituent (e.g., a gene product such as an RNA species, e.g., mRNA or miRNA, or a protein molecule) present in one or more cells, or an average amount of a cellular constituent present across multiple cells. When referring to mRNA or protein expression, the term generally refers to the amount of any RNA or protein species corresponding to a particular genomic locus, e.g., a particular gene. However, in some embodiments, an abundance can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Thus, a “tumor sample” refers to a biological sample obtained or derived from a tumor of a subject, as described herein.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

As used herein, the terms “genomic alteration,” “mutation,” and “variant” interchangeably refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “germline variants” refers to genetic variants inherited from maternal and paternal DNA. Germline variants may be determined through a matched tumor-normal calling pipeline.

As used herein, unless otherwise dictated by context “nucleotide” or “nt” refers to ribonucleotide.

As used herein, the terms “patient” and “subject” are used interchangeably, and may be taken to mean any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that vary in size from tens to hundreds to thousands of base pairs. Illumina parallel sequencing can provide sequence reads that vary to a lesser extent (e.g., where most sequence reads are of a length of about 200 bp or less). A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes (e.g., in hybridization arrays or capture probes) or amplification techniques, such as the polymerase chain reaction (PCR) or linear amplification using a single primer or isothermal amplification.

As disclosed herein, the terms “sequencing,” “sequence determination,” and the like refer generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “somatic variants” refers to variants arising as a result of dysregulated cellular processes associated with neoplastic cells, e.g., a mutation. Somatic variants may be detected via subtraction from a matched normal sample.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest on one or more chromosomes. In some embodiments, in addition to loci that are informative for precision oncology, a targeted panel includes one or more probes for sequencing one or more of a loci associated with a different medical condition, a loci used for internal control purposes, or a loci from a pathogenic organism (e.g., an oncogenic pathogen).

As used herein, the term “tumor fraction” refers to the fraction of nucleic acid molecules in a sample that originates from a cancerous tissue of the subject, rather than from a noncancerous tissue (e.g., a germline or hematopoietic tissue).

As used herein, the terms “variant” or “mutation” refer to a detectable change in the genetic material of one or more cells. A variant or mutation can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene or a large span of a chromosome) (CNV), a partial or complete change in the ploidy of the cell, and/or changes in the epigenetic information of a genome, such as altered DNA methylation patterns. For example, a single nucleotide variant or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.” In some embodiments, a variant is a change in the genetic information of the cell relative to a particular reference genome or one or more “normal” or “reference” alleles found in the population of the species of the subject. In some embodiments, a variant is a change in the genetic information of the cell relative to a reference cell or tissue, such as a “normal” or “healthy” tissue in the subject. In some embodiments, a variant is a germline mutation or a somatic mutation.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

### 1. Exemplary System Embodiments for Check Point Identification.

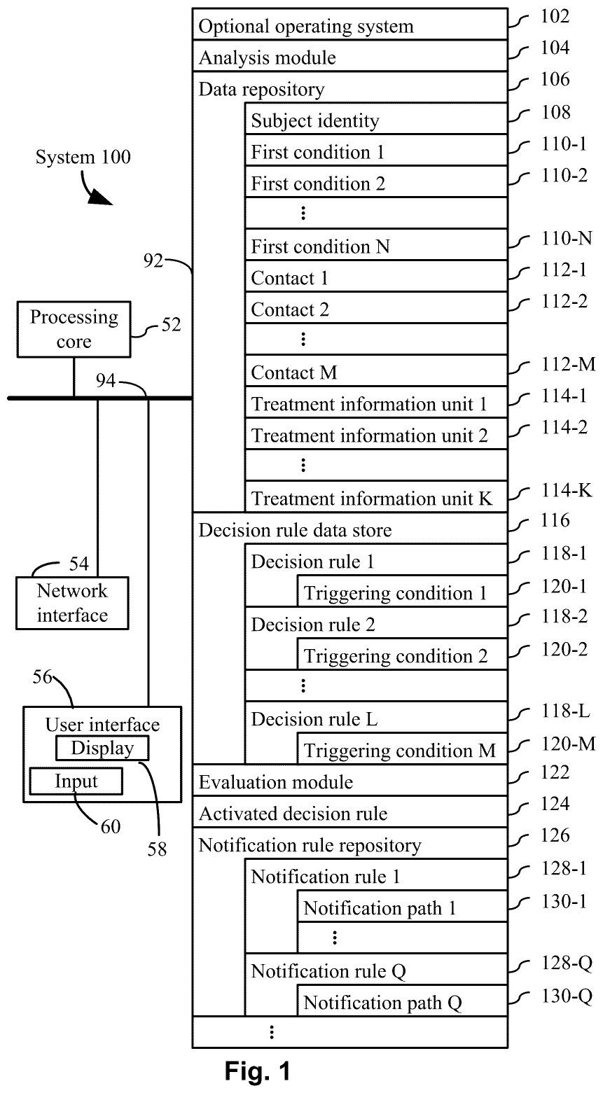

Now that an overview of some aspects of the present disclosure and some definitions used in the present disclosure have been provided, details of an exemplary system are described in conjunction with FIG. 1.

FIG. 1 illustrates a computer system 100 for check point identification. In typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines and/or distributed collection of servers that host software and infrastructure accessed over the internet via a cloud computing system. One of skill in the art will appreciate that a wide array of different computer topologies is possible for the computer system 100 and all such topologies are within the scope of the present disclosure.

Turning to FIG. 1 with the foregoing in mind, the computer system 100 comprises one or more processing units (CPUs) 52, a network or other communications interface 54, a user interface 56 (e.g., including an optional display 58 and optional input 60 (e.g. keyboard or other form of input device)), a memory 92 (e.g., random access memory, persistent memory, or combination thereof), and one or more communication busses 94 for interconnecting the aforementioned components. To the extent that components of memory 92 are not persistent, data in memory 92 can be seamlessly shared with non-volatile memory (not shown) or portions of memory 92 that are non-volatile/persistent using known computing techniques such as caching. Memory 92 can include mass storage that is remotely located with respect to the central processing unit(s) 52. In other words, some data stored in memory 92 may in fact be hosted on computers that are external to computer system 100 but that can be electronically accessed by the computer system 100 over an Internet, intranet, or other form of network or electronic cable using network interface 54. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 102 that includes procedures for handling various basic system services;
an analysis module 104 for check point identification;
a data repository 106 associated with a subject that comprises:
   a plurality of treatment information units 114-1, 114-2, . . . , 114-K, where K is a positive integer of 3 or greater, and
   an identity of the subject 108
   one or more first conditions 110-1, 110-2, . . . , 110-N, where N is a positive integer and where the one or more first conditions may be found within the treatment information units 114;
   one or more contact 112-1, 112-2, . . . , 112-M, where M is a positive integer and where the one or more contacts be found within the treatment information units 114;
a decision rule data store 116 comprising
   a plurality of decision rules 118-1, 118-2, . . . , 118-L, wherein L is a positive integer of 3 or greater, where at least a subset of the decision rules each have a corresponding triggering condition 120 in a set of triggering conditions 120-1, 120-2, . . . , 120-M, wherein M is a positive integer of 3 or greater;
an evaluation module 122 for activating, under specific conditions, a first decision rule 118 (activated decision rule 124) whose corresponding triggering condition 120 arises in the data repository 106;
a notification rule repository 126 that comprises:
   a plurality of notification rules 128 (e.g., 128-1, . . . , 128-Q, where Q is a positive integer), and for each respective notification rule 128, a corresponding notification path 130 (e.g., 130-1, . . . , 130-Q, where Q is a positive integer) that consists of a subset of the contacts 112.

In some embodiments, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 optionally stores a subset of the modules and data structures identified above. Furthermore, in some embodiments the memory 92 stores additional modules and data structures not described above.

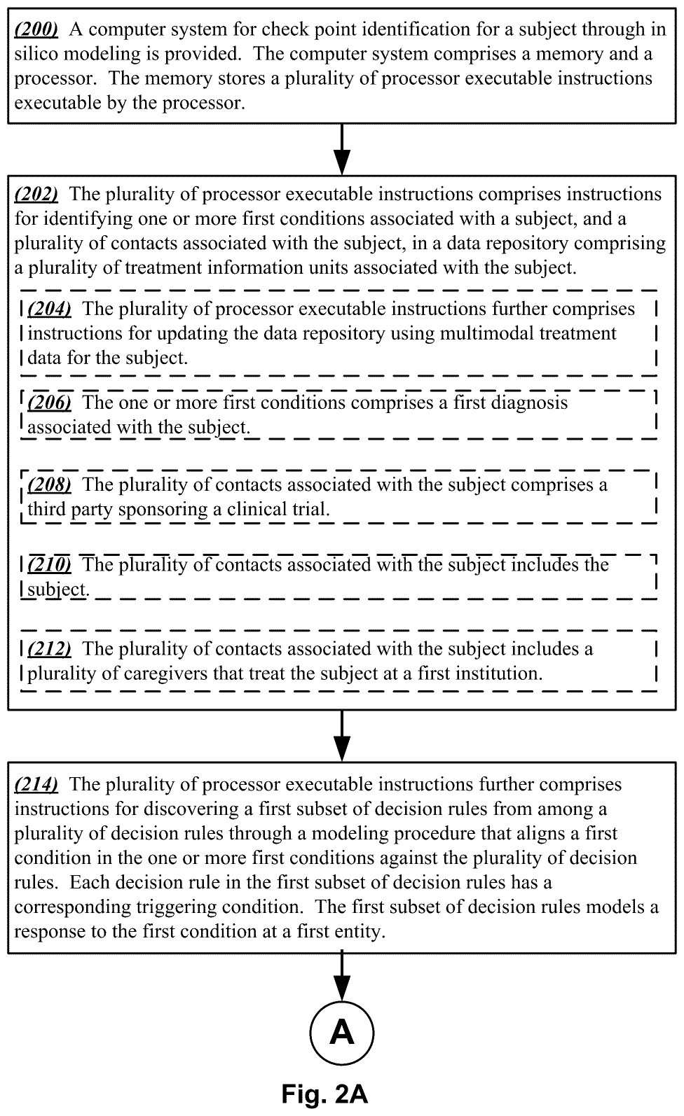

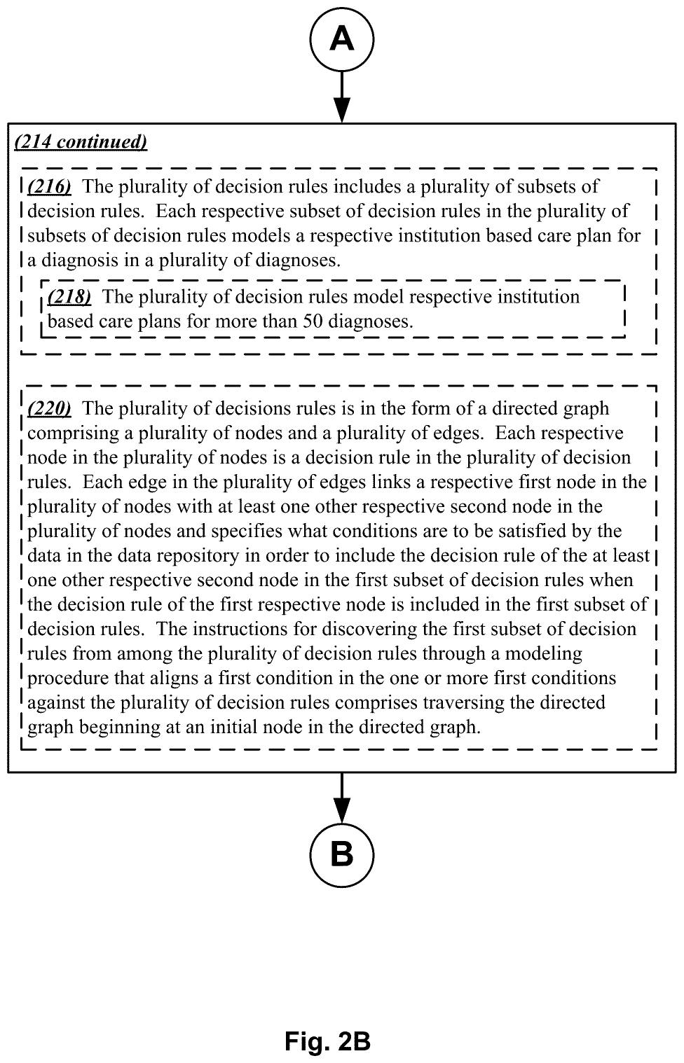

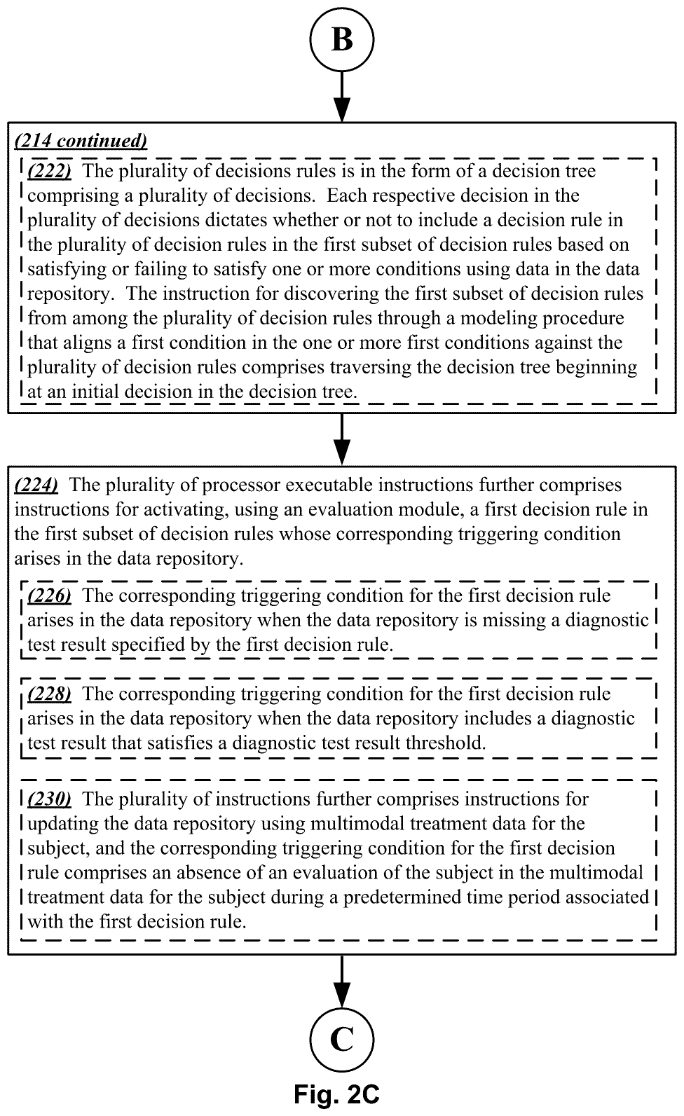

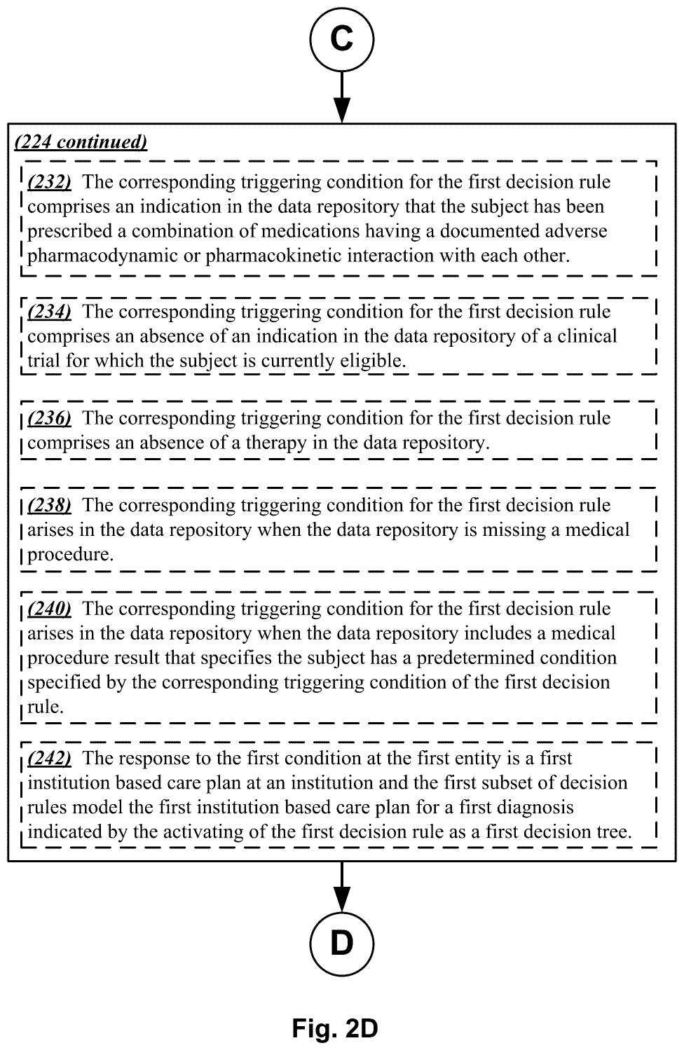

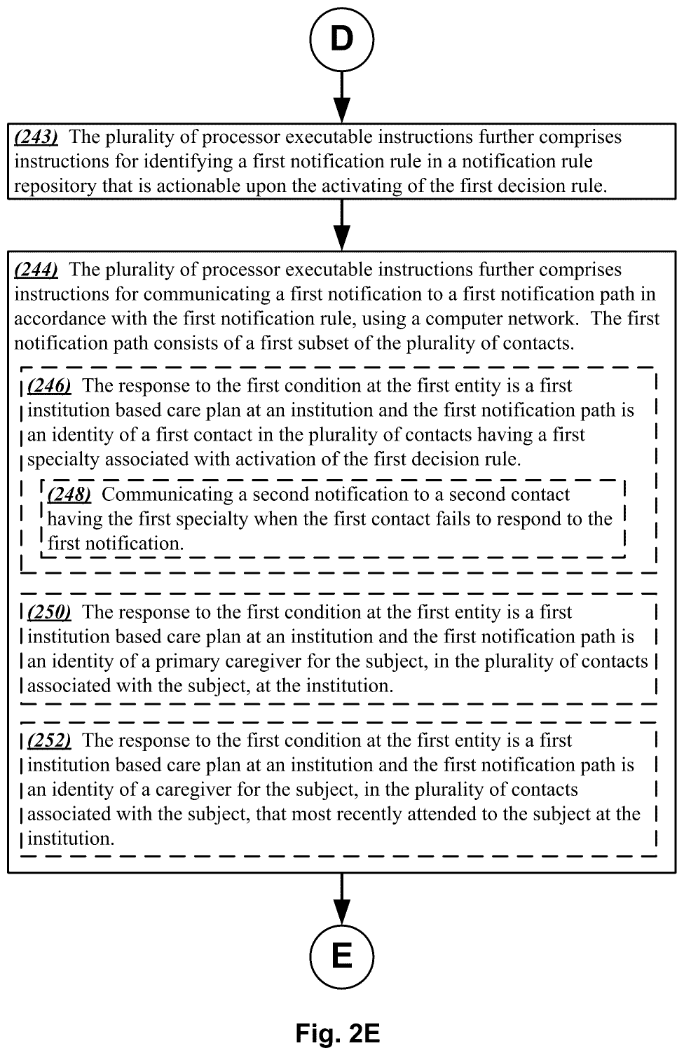

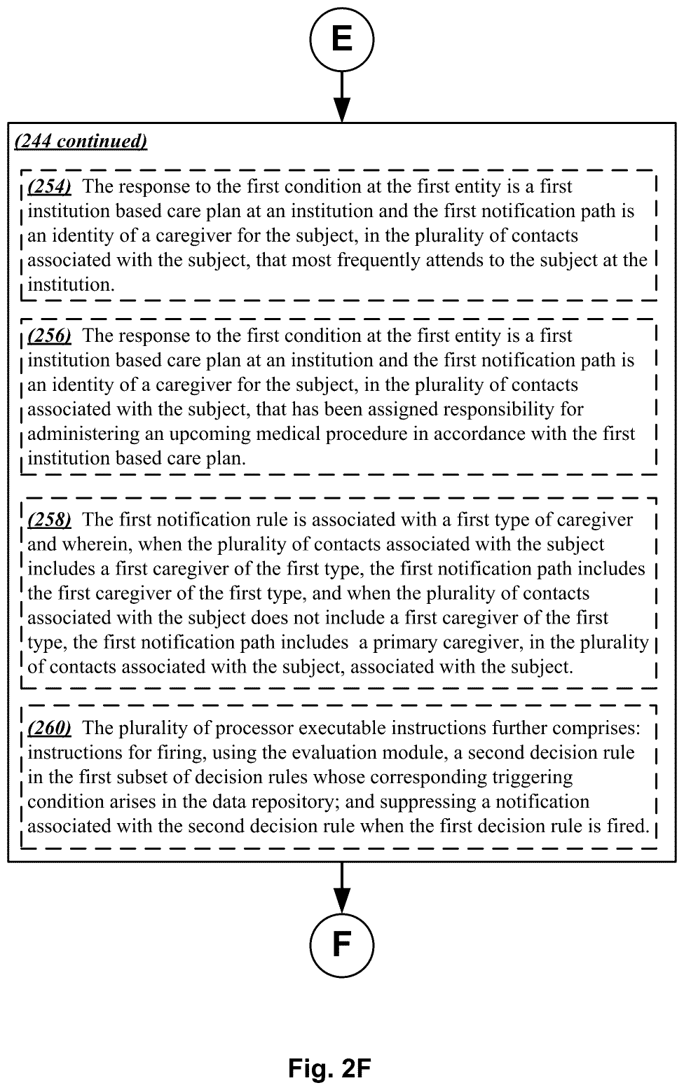

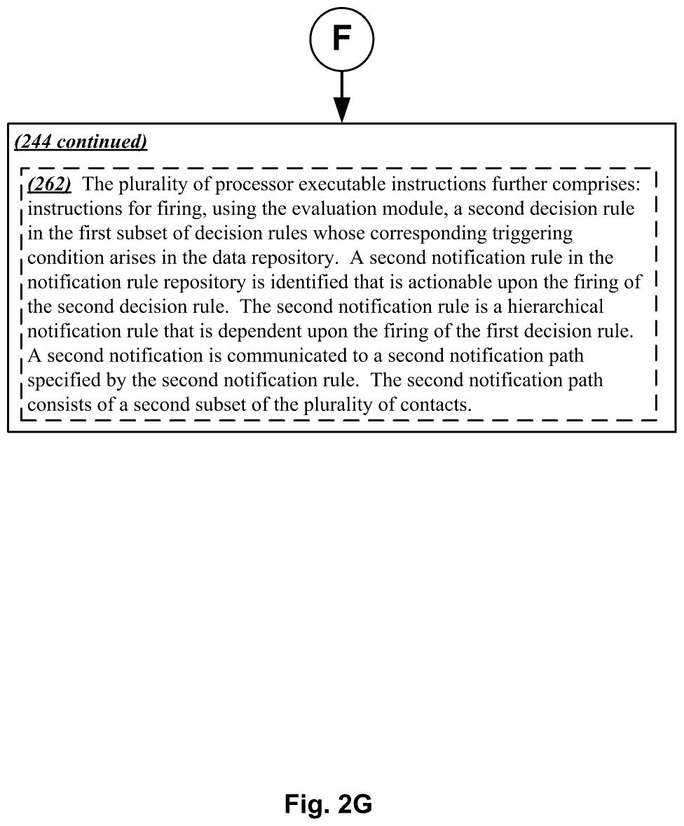

Now that a system for check point identification, the detailed instructions executed by such a system are detailed with reference to FIG. 2 as discussed below.

### II. Instructions Executed by a First Embodiment of a Check Point Identification System.

Block 200. Referring to block 200, such as computer system 100 of FIG. 1, for check point identification for a subject through in silico modeling is provided. The computer system 100 comprises a memory 92 and a 54 processor. The memory 92 stores a plurality of processor executable instructions executable by the processor.

Block 202. Referring to block 202, the plurality of processor executable instructions comprises instructions for identifying one or more first conditions 110 associated with a subject, and a plurality of contacts 112 associated with the subject, in a data repository 106 comprising a plurality of treatment information units 114 associated with the subject.

In some embodiments the plurality of treatment information units 114 comprises an electronic health record of the subject. In some embodiments the plurality of treatment information units 114 comprises family history of the subject, past medical treatments in incurred by the subject, subject surgical history, medications that subject has taken in the past including dosages, medications that subject is currently taking including dosages, and/or a log of medical consultations (including, but not limited to, in person medical consultations, remote video/telemedicine consultations, and telephonic consultations)

In some embodiments the plurality of treatment information units 114 comprises one or more echocardiography (Echo) test results, one or more electrocardiograms (ECG), one or more computed tomography (CT) scans, and/or one or more magnetic resonance images (MRI) taken of the subject or of a region of the subject or of an organ of the subject (e.g, brain, heart, lung, colon, etc.).

In some embodiments, the plurality of treatment information units 114 comprises test results of tumor (e.g., xT) or liquid biopsy (e.g., xF) data. The xT and xF assays are described in Beaubier et al., 2019, “Clinical validation of the tempus xT next-generation targeted oncology sequencing assay,” Oncotarget 10, pp. 2384-2396, which is hereby incorporated by reference.

In some embodiments, the plurality of treatment information units 114 comprises test results such as:
white blood cell count (e.g., in units of count per microliter of blood),
red blood cell count (e.g., in units of count per microliter of blood),
amount of hemoglobin (e.g., in units of gram per deciLiter of blood),
percent hematocrit in blood,
mean corpuscular volume (MCV) in units of femtoliters,
mean corpuscular hemoglobin (MCH) (e.g., in units of picograms (pg)),
1-3-beta-D-Glucan level (measures the level of beta-D-glucan in the blood, which is a polysaccharide found in the cell walls of certain fungi and yeasts; this test is used to help diagnose and monitor fungal infections, particularly in immunocompromised patients in some embodiments),
1917 rapid plasma regain (RPR; a screening test for syphilis that detects nonspecific antibodies that may indicate a syphilis infection in some embodiments),
alpha-1 antitrypsin levels (A1AT; a protein produced by the liver that protects the lungs from inflammation caused by infection or irritants such as tobacco smoke, in which low levels can indicate a deficiency, which can lead to lung and liver disease),
A1AT mutation (e.g., genetic testing for mutations in the gene that codes for alpha-1 antitrypsin, mutations can result in deficient or dysfunctional alpha-1 antitrypsin, increasing the risk of emphysema and liver disease),
arterial blood gas-potassium (ABG K; measures the levels of various gases and pH in arterial blood, providing information on lung function and acid-base balance; potassium is one of the electrolytes measured and is crucial for proper cell function, particularly in the heart and muscles),
absolute lymphocytes (the count of lymphocytes, a type of white blood cell, in the blood; lymphocytes play a role in the immune system, and abnormal levels can indicate infection, inflammation, or other immune-related conditions),
absolute neutrophils (the count of neutrophils, another type of white blood cell, in the blood; neutrophils are involved in fighting bacterial infections; abnormal levels can signal infection, inflammation, or bone marrow issues),
acanthocyte (a type of red blood cell with spiked cell membranes; the presence of acanthocytes can indicate various conditions, such as liver disease, neuroacanthocytosis, or abetalipoproteinemia in some embodiments),
angiotensin-converting enzyme (ACE) levels (an enzyme that converts angiotensin I to angiotensin II, which regulates blood pressure; elevated levels can be seen in sarcoidosis and other conditions),
adrenocorticotropic hormone (ACTH) levels (ACTH is a hormone produced by the pituitary gland that stimulates the adrenal glands to release cortisol; it is used, for example, to diagnose adrenal and pituitary disorders in some embodiments),
adenovirus DNA quantity (a test that quantifies the amount of adenovirus DNA in blood, used to diagnose and monitor adenovirus infections, such as immunocompromised individuals in some embodiments),
adiponectin levels (adiponectin is a protein hormone involved in regulating glucose levels and fatty acid breakdown; this test measures its levels, which can be associated with metabolic syndrome, type 2 diabetes, and cardiovascular disease in some embodiments),
adjusted calcium (a calculation that corrects total calcium levels based on the amount of albumin in the blood; this is useful, for example, for accurately assessing calcium status, such as patients with abnormal albumin levels in some embodiments),
alpha-fetoprotein (AFP) levels (a protein produced by the liver and yolk sac of a fetus; elevated levels in adults can indicate liver cancer, germ cell tumors, or other malignancies; it is also used in prenatal screening for certain developmental disorders in some embodiments),
anion gap (AGAP; a calculated value based on the concentrations of sodium, potassium, chloride, and bicarbonate in the blood; it helps to identify the cause of metabolic acidosis in some embodiments),
anti-glial nuclear antibody 1 (AGNA-1) levels (an antibody that targets glial cells in the nervous system; the presence of these antibodies can indicate autoimmune neurological disorders, such as paraneoplastic syndromes, in some embodiments),
albumin by serum protein electrophoresis (Alb SPE; a test that measures albumin levels in the blood and helps to detect and monitor diseases affecting the liver, kidneys, and other conditions affecting protein levels in some embodiments),
albumin (a protein made by the liver that helps to maintain osmotic pressure and the transporting of various substances in the blood; low levels can indicate liver disease, kidney disease, or malnutrition),
albumin serum-quest (a specific test to measure serum albumin levels, often used to assess overall health, nutritional status, and liver or kidney function),
aldolase (an enzyme involved in breaking down glucose to produce energy; elevated levels can indicate muscle damage, liver disease, or other conditions affecting the muscles or liver in some embodiments),
aldosterone-Mayo (measures the amount of aldosterone, a hormone produced by the adrenal glands that helps regulate blood pressure by controlling sodium and potassium levels; used to diagnose conditions like hyperaldosteronism or adrenal insufficiency in some embodiments),
alkaline phosphatase (Alk Phos) level (an enzyme found in the liver, bones, kidneys, and digestive system; elevated levels can indicate liver disease, bone disorders, or bile duct obstruction in some embodiments),
total alkaline phosphatase level (similar to Alk Phos, this measures the total amount of alkaline phosphatase enzyme in the blood, which can help diagnose liver and bone diseases in some embodiments),
alpha-1 antitrypsin (A1AT) level (measures the concentration of alpha-1 antitrypsin, a protein produced by the liver that helps protect tissues from damage caused by enzymes; it is particularly important in protecting the lungs from damage by neutrophil elastase),
alpha-1-antitrypsin phenotype-Quest (a test that determines the specific genetic variants of the alpha-1 antitrypsin protein, helping to diagnose A1AT deficiency and assess risk for related diseases in some embodiments),
alpha-2 macroglobulin level (Alpha2; a protein involved in inhibiting enzymes that break down proteins; elevated levels can be seen in nephrotic syndrome and other conditions in some embodiments),
alpha-2-macroglobu-Quest (measures the level of alpha-2 macroglobulin in the blood, used to evaluate liver disease, nephrotic syndrome, and other disorders, in some embodiments),
alpha-tocopherol level (Alpha Toco; another name for Vitamin E, an antioxidant that protects cells from damage; this test measures vitamin E levels to assess nutritional status and antioxidant capacity, in some embodiments),
alanine aminotransferase (ALT; an enzyme found in the liver, elevated levels indicate liver damage or disease in some embodiments),
ALT-Quest (a specific test to measure ALT levels, used to diagnose and monitor liver conditions in some embodiments),
anti-mitochondrial antibodies (AMA; autoantibodies targeting mitochondria, used to diagnose primary biliary cholangitis, an autoimmune liver disease, in some embodiments),
amiodarone level (a medication used to treat and prevent various types of irregular heartbeats in some embodiments, monitoring amiodarone blood levels helps manage dosage and detect potential toxicity in some embodiments),
amphiphysin Ab-Mayo (tests for antibodies against amphiphysin, associated with paraneoplastic neurological syndromes and some autoimmune disorders in some embodiments),
amylase levels (an enzyme that helps digest carbohydrates; elevated levels can indicate pancreatitis, pancreatic injury, or other pancreatic diseases in some embodiments),
antinuclear antibodies (ANA; autoantibodies that target the cell nucleus; positive results can indicate autoimmune diseases like lupus, rheumatoid arthritis, and others in some embodiments),
ANA Pattern (describes the staining pattern of antinuclear antibodies in some embodiments, providing information regarding specific autoimmune diseases in some embodiments),
ANA Quantitative (ANA Qnt; measures the concentration of antinuclear antibodies in the blood, used to diagnose and monitor autoimmune diseases, in some embodiments),
#ANA titer (indicates the dilution level at which ANA is still detectable, with higher titers suggesting a higher level of autoimmunity),
cytoplasmic anti-neutrophil cytoplasmic antibodies (ANCA-C; autoantibodies targeting proteins in the cytoplasm of neutrophils, associated with granulomatosis with polyangiitis (Wegener's granulomatosis);
perinuclear anti-neutrophil cytoplasmic antibodies (ANCA-P; autoantibodies targeting proteins around the nucleus of neutrophils, associated with microscopic polyangiitis and other vasculitides),
anisocytosis (refers to the presence of red blood cells of unequal sizes, often indicating anemia or other blood disorders),
anti-neuronal nuclear antibody 1 levels (ANNA-1; autoantibodies associated with paraneoplastic neurological syndromes, targeting neurons),
ANNA-2 levels (similar to ANNA-1, these autoantibodies target different neuronal antigens and are associated with neurological paraneoplastic syndromes),
ANNA-3 (another variant of anti-neuronal nuclear antibodies, associated with different paraneoplastic syndromes),
anti-cyclic citrullinated peptide antibodies (Anti-CCP (IgG); autoantibodies strongly associated with rheumatoid arthritis, helping in diagnosis and prognosis),
anti-double stranded DNA antibodies levels (Anti-dsDNA; autoantibodies that target the double-stranded DNA in cells; high levels are specific for systemic lupus erythematosus),
anti-Jo-1 Ab levels (Anti-Jo-1 antibody; autoantibodies against the Jo-1 antigen, associated with polymyositis and dermatomyositis, types of inflammatory myopathies in some embodiments),
anti-PM/Scl Ab (anti-PM/Scl antibody; autoantibodies against PM/Scl antigens, associated with polymyositis, systemic sclerosis, and overlap syndromes in some embodiments),
anti-Smith (Anti-Smith Antibody; autoantibodies targeting the Smith antigen, highly specific for systemic lupus erythematosus;
Anti-SS-A 52 kD Ab (Anti-SSA/Ro 52 kD Antibody; autoantibodies targeting the 52 kD protein of the SSA/Ro antigen, associated with Sjogren's syndrome and systemic lupus erythematosus in some embodiments),
IgG (Immunoglobulin G; a type of antibody in blood and body fluids, involved in fighting bacterial and viral infections, abnormal levels can indicate immune deficiencies, infections, or autoimmune diseases in some embodiments),
anti-TPO (anti-thyroid peroxidase antibody; autoantibodies against thyroid peroxidase, often elevated in autoimmune thyroid diseases such as Hashimoto's thyroiditis and Graves' disease),
anti-U1-RNP Ab (anti-U1 ribonucleoprotein antibody; autoantibodies targeting U1-RNP, associated with mixed connective tissue disease and other autoimmune conditions),
anti-Xa (Anti-Factor Xa; a test that measures the activity of anticoagulants like heparin in the blood, used to monitor anticoagulant therapy),
APCR (activated protein C resistance; a test that assesses resistance to activated protein C, often associated with factor V Leiden mutation, increasing the risk of blood clots in some embodiments),
apoliprotein A1 (measures levels of apolipoprotein A1, a component of HDL cholesterol; low levels can indicate increased cardiovascular risk in some embodiments);
ASMA (anti-smooth muscle antibody; autoantibodies targeting smooth muscle, associated with autoimmune hepatitis and other autoimmune diseases in some embodiments),
Asper Galact (Aspergillus Galactomannan Antigen; a test that detects galactomannan, a component of Aspergillus cell walls, used to diagnose invasive aspergillosis in some embodiments),
Aspergillus flavus Ab (measures antibodies against Aspergillus flavus, used to diagnose infections caused by this fungus in some embodiments),
Aspergillus fumigatus Ab (measures antibodies against Aspergillus fumigatus, used to diagnose infections caused by this fungus in some embodiments),
Aspergillus niger Ab (measures antibodies against Aspergillus niger, used to diagnose infections caused by this fungus in some embodiments),
AST (Aspartate Aminotransferase; an enzyme found in the liver and other tissues; elevated levels can indicate liver damage, muscle damage, or other conditions in some embodiments),
AT levels (Antithrombin; a protein that helps regulate blood clotting; low levels can increase the risk of thrombosis in some embodiments),
Atyp Lymph (atypical lymphocytes; abnormal lymphocytes that can indicate viral infections, autoimmune disorders, or blood cancers in some embodiments),
Bahia Grass (g17) Class (allergy testing for specific IgE antibodies to Bahia grass pollen, used to diagnose grass pollen allergies in some embodiments),
Bahia Grass (g17) (measures the level of IgE antibodies specific to Bahia grass pollen in some embodiments),
Bands (immature white blood cells released during infection or inflammation, high levels can indicate a severe infection or bone marrow disorder in some embodiments),
Basophil levels (a type of white blood cell involved in allergic reactions and inflammation; abnormal levels can indicate allergic responses, infections, or bone marrow disorders in some embodiments),
BCLL Final Dx-Mayo (B-Cell Chronic Lymphocytic Leukemia Final Diagnosis; the final diagnosis report for B-cell chronic lymphocytic leukemia, provided by the Mayo Clinic),
BCLL Result-Mayo (the result of tests related to B-cell chronic lymphocytic leukemia, provided by the Mayo Clinic),
BCLL Spec-Mayo (a specific testing and results related to B-cell chronic lymphocytic leukemia, provided by the Mayo Clinic),
B.E. ABG (Base Excess in Arterial Blood Gas; a measurement from an arterial blood gas test that indicates the amount of excess or deficient base in the blood, used to assess acid-base balance, in some embodiments),
B.E. CVL (base Excess in central venous line; similar to B.E. ABG, but measured from a central venous line, providing information on the patient's acid-base balance in some embodiments),
B.E. ECMO Post ABG (base excess measurement from an arterial blood gas test after extracorporeal membrane oxygenation [ECMO] treatment, used to monitor acid-base status post-treatment),
B.E. ECMO PRE ABG (base excess measurement from an arterial blood gas test before ECMO treatment, used to assess acid-base status before treatment in some embodiments),
B.E. MV (base excess in mechanical ventilation; base excess measurement in patients undergoing mechanical ventilation, used to monitor and manage acid-base balance in some embodiments),
Beta cell levels, beta2-Glycoprotein I (IgG) (measures IgG antibodies against beta-2 glycoprotein I, associated with antiphospholipid syndrome, which increases the risk of blood clots in some embodiments),
Beta2-Glycoprotein I (IgM) (measures IgM antibodies against beta-2 glycoprotein I, also associated with antiphospholipid syndrome in some embodiments),
Beta 2 Micro (Beta-2 Microglobulin; a protein found on the surface of many cells; elevated levels can indicate kidney disease, inflammation, or certain cancers such as multiple myeloma in some embodiments),
Beta-2 Microglobulin Random Urine-Quest (Measures the level of beta-2 microglobulin in a random urine sample; elevated levels can indicate kidney damage or disease, and it's used to monitor conditions such as multiple myeloma and chronic kidney disease),
Beta Gamma Toco (measures beta and gamma-tocopherols, which are forms of Vitamin E; these antioxidants are measured to assess nutritional status and oxidative stress in some embodiments),
Beta-Hydroxybut-Mayo (Beta-Hydroxybutyrate; a ketone body measured in the blood, used to diagnose and monitor diabetic ketoacidosis and other conditions involving abnormal ketone levels in some embodiments),
BF WBC Ct (Body Fluid White Blood Cell Count): measures the number of white blood cells in body fluids (e.g., cerebrospinal fluid, pleural fluid) to detect infections, inflammation, or other conditions in some embodiments),
B. henselae IgG Screen-Quest (Bartonella henselae IgG Screen): a blood test that screens for IgG antibodies against Bartonella henselae, the bacteria responsible for cat scratch disease,
B. henselae IgG Titer-Quest (Bartonella henselae IgG Titer): measures the level of IgG antibodies against Bartonella henselae, used to confirm and monitor infection in some embodiments,
B. henselae IgM Screen-Quest (Bartonella henselae IgM Screen): a blood test that screens for IgM antibodies against Bartonella henselae, indicating a recent or current infection in some embodiments,
B. hens. IgG Reflex-Quest (Bartonella henselae IgG Reflex): a reflex test performed if the initial B. henselae IgG screen is positive, providing additional confirmation and quantification in some embodiments,
B. hens. IgM Reflex-Quest (Bartonella henselae IgM Reflex): a reflex test performed if the initial B. henselae IgM screen is positive, providing additional confirmation and quantification in some embodiments,
bicarbonate: measures the level of bicarbonate in the blood, which helps maintain the acid-base balance; abnormal levels can indicate metabolic acidosis or alkalosis in some embodiments,
bile acids: measures the concentration of bile acids in the blood; elevated levels can indicate liver dysfunction or bile flow obstruction in some embodiments,
Bili Direct (Direct Bilirubin): measures the level of direct (conjugated) bilirubin in the blood; elevated levels can indicate liver disease, bile duct obstruction, or other conditions affecting bilirubin metabolism in some embodiments,
Bili Indirect (Indirect Bilirubin): measures the level of indirect (unconjugated) bilirubin in the blood; elevated levels can indicate hemolysis or disorders of bilirubin metabolism in some embodiments,
Bili Total (Total Bilirubin): measures the total amount of bilirubin (both direct and indirect) in the blood; used to assess liver function and diagnose jaundice in some embodiments,
BK Virus Quantitative: a test that quantifies the amount of BK virus DNA in the blood, used to monitor BK virus infection, particularly in immunocompromised patients such as those who have had a kidney transplant,
BK Virus Urine-Quest: measures the presence and amount of BK virus DNA in the urine, used to diagnose and monitor BK virus infection in some embodiments,
Blastomyces Ag-Quest (Blastomyces Antigen): tests for the presence of antigens from Blastomyces dermatitidis, the fungus that causes blastomycosis, in blood, urine, or other body fluids,
Blastomyces Interp-Quest (Blastomyces Interpretation): provides the interpretation of the Blastomyces antigen test results, helping to diagnose blastomycosis,
Blasts: measures immature white blood cells found in the bone marrow; the presence of blasts in the peripheral blood can indicate leukemia or other bone marrow disorders in some embodiments,
Basic Metabolic Panel (BMP) measures glucose, calcium, electrolytes, and kidney function markers,
BNP CHF (B-Type Natriuretic Peptide for Congestive Heart Failure): measures BNP levels in the blood, which are elevated in heart failure, used to diagnose and assess the severity of heart failure in some embodiments,
BR 15-3 (CA 15-3): a tumor marker used primarily to monitor breast cancer treatment and detect recurrences,
BSA (Body Surface Area): a calculated measurement of the total surface area of the human body, often used to determine medication dosages and assess physiological functions,
BUN (Blood Urea Nitrogen): measures the amount of nitrogen in the blood from urea, a waste product; elevated levels can indicate kidney dysfunction or dehydration, in some embodiments,
Burr Cells: red blood cells with irregular, spiked cell membranes; their presence can indicate kidney disease, liver disease, or other conditions in some embodiments,
C3c (Complement Component 3c): part of the complement system involved in immune responses; abnormal levels can indicate autoimmune diseases, infections, or other inflammatory conditions in some embodiments,
C4c (Complement Component 4c): another part of the complement system; abnormal levels can help diagnose and monitor autoimmune diseases and other inflammatory conditions in some embodiments,
calcium: measures the level of calcium in the blood; abnormal levels can indicate parathyroid disorders, bone diseases, kidney disease, or other conditions in some embodiments,
Calc U Alb (Calculated Urinary Albumin): measures the amount of albumin in the urine, used to assess kidney function and detect early kidney damage in some embodiments,
Calc U Alb/Creat (calculated urinary albumin/creatinine ratio): a ratio of urinary albumin to creatinine, used to detect and monitor kidney disease in some embodiments,
Calc U Ca (Calculated Urinary Calcium): measures the amount of calcium excreted in the urine, used to diagnose and monitor conditions affecting calcium metabolism, in some embodiments,
Calc U Creat (Calculated Urinary Creatinine): measures creatinine levels in urine, used to assess kidney function in some embodiments,
Calc U Prot (Calculated Urinary Protein): measures the total amount of protein in urine, used to diagnose kidney disease in some embodiments,
Calc U Prot/Creat (Calculated Urinary Protein/Creatinine Ratio): a ratio of urinary protein to creatinine, used to detect and monitor kidney disease in some embodiments,
Calc Ur TP Elect (Calculated Urinary Total Protein Electrophoresis): analyzes the different proteins in the urine, used to diagnose and monitor kidney disease and other conditions in some embodiments,
Calc U Urea (Calculated Urinary Urea): measures the amount of urea excreted in the urine, used to assess kidney function and nitrogen balance in some embodiments,
CASPR2-IgG-Mayo (Contactin-Associated Protein-Like 2 IgG Antibody): tests for antibodies against CASPR2, associated with autoimmune neurological disorders in some embodiments,
CBC/Diff Performed at: Complete Blood Count with Differential, where “Performed at” indicates the location where the test was conducted, measures different components of the blood, including red and white blood cells and platelets,
CBC Performed at: Complete Blood Count, where “Performed at” indicates the location where the test was conducted, measures the overall health and detects a variety of disorders, such as anemia, infection, and many other diseases in some embodiments,
CDC Hep C Ab (Centers for Disease Control Hepatitis C Antibody): gests for antibodies against the hepatitis C virus, indicating exposure to the virus in some embodiments,
CDC Hep C Ab Indications: provides the clinical indications or reasons for performing the hepatitis C antibody test, such as risk factors or symptoms in some embodiments,
CDC HIV (Centers for Disease Control HIV Test): tests for HIV antibodies and antigens to diagnose HIV infection in some embodiments,
CEAg (Carcinoembryonic Antigen): a tumor marker used to monitor colorectal cancer and other cancers; elevated levels can indicate cancer, but can also be elevated in non-cancerous conditions in some embodiments,
Chlamydia pneumoniae: tests for antibodies (IgG, IgM) against Chlamydia pneumoniae, a bacterium that causes respiratory infections like pneumonia and bronchitis,
chloride: measures the level of chloride in the blood, an important electrolyte that helps maintain acid-base balance, fluid balance, and muscle and nerve function,
Chol (cholesterol): measures the total cholesterol level in the blood; high levels can indicate an increased risk of cardiovascular disease,
Chromogranin A, Serum: a protein found in neuroendocrine cells; elevated levels can indicate neuroendocrine tumors, such as pheochromocytomas, carcinoid tumors, and neuroblastomas,
CMP Performed at: comprehensive Metabolic Panel (CMP) measures glucose, calcium, proteins, electrolytes, and kidney and liver function markers, where “Performed at” indicates the location where the test was conducted,
CMV IgG (Cytomegalovirus IgG): tests for IgG antibodies against cytomegalovirus, indicating past or latent infection,
CMV IgM (Cytomegalovirus IgM): tests for IgM antibodies against cytomegalovirus, indicating recent or current infection,
CMVQNTX (cytomegalovirus quantitative PCR): measures the amount of cytomegalovirus DNA in the blood, used to monitor active CMV infection, especially in immunocompromised patients,
COHb % ABG (Carboxyhemoglobin percentage in arterial blood gas): measures the percentage of hemoglobin that is bound to carbon monoxide in arterial blood, used to diagnose carbon monoxide poisoning,
COHb % VBG (carboxyhemoglobin percentage in venous blood gas): measures the percentage of hemoglobin that is bound to carbon monoxide in venous blood, also used to diagnose carbon monoxide poisoning,
Comment IFE (Immunofixation Electrophoresis Comment): provides additional interpretation or comments on the results of immunofixation electrophoresis, a test used to identify abnormal proteins in the blood in some embodiments,
Comment SPE (Serum Protein Electrophoresis Comment): provides additional interpretation or comments on the results of serum protein electrophoresis, a test used to separate and measure proteins in the blood in some embodiments,
Comment U IFE Random (Urine Immunofixation Electrophoresis Random Comment): provides additional interpretation or comments on the results of random urine immunofixation electrophoresis, used to identify abnormal proteins in the urine in some embodiments,
Comment U PEP Random (Urine Protein Electrophoresis Random Comment) provides additional interpretation or comments on the results of random urine protein electrophoresis, used to analyze the proteins in the urine,
Comment U PEP Timed (Urine Protein Electrophoresis Timed Comment): provides additional interpretation or comments on the results of timed urine protein electrophoresis, used to analyze the proteins in the urine over a specific time period,
Comment Ur IFE Timed (Urine Immunofixation Electrophoresis Timed Comment): provides additional interpretation or comments on the results of timed urine immunofixation electrophoresis, used to identify abnormal proteins in the urine over a specific time period,
Copper Serum: measures the level of copper in the blood, used to diagnose and monitor conditions such as Wilson's disease and copper deficiency,
Cort Baseline (Cortisol Baseline): measures the baseline level of cortisol in the blood, used to assess adrenal gland function,
Cortisol 30 (Cortisol 30 minutes post-stimulation): measures the level of cortisol in the blood 30 minutes after a stimulation test, used to assess adrenal gland function and diagnose conditions like Addison's disease or Cushing's syndrome,
Cortisol 60 (Cortisol 60 minutes post-stimulation): measures the level of cortisol in the blood 60 minutes after a stimulation test, also used to assess adrenal gland function,
Cortisol Level: measures the level of cortisol in the blood, which is a hormone produced by the adrenal glands, it helps diagnose conditions like Addison's disease, Cushing's syndrome, and adrenal insufficiency, in some embodiments,
COVID-19 IgG: tests for IgG antibodies against the SARS-CoV-2 virus, indicating a past infection or response to vaccination in some embodiments,
COVID-19 Performed at: indicates the location where the COVID-19 test was performed,
COVID-19 (SARS-CoV-2) Ag/FIA (Antigen/Fluorescent Immunoassay): a test that detects specific antigens from the SARS-CoV-2 virus, used for diagnosing active COVID-19 infection in some embodiments,
COVID-19 (SARS-CoV-2) PCR: a polymerase chain reaction (PCR) test that detects the genetic material of the SARS-CoV-2 virus, used for diagnosing active COVID-19 infection in some embodiments,
COVID-19 Specimen Type: indicates the type of specimen collected for COVID-19 testing, such as nasal swab, saliva, or blood,
Coxsackie A-Abs-Quest (Coxsackie A Antibodies): tests for antibodies against Coxsackie A virus, which can cause hand, foot, and mouth disease, as well as other illnesses,
C-Peptide: Measures the level of C-peptide in the blood, which is a byproduct of insulin production; it helps assess insulin production and differentiate between type 1 and type 2 diabetes in some embodiments,
C Prot (C-Reactive Protein, CRP): measures the level of CRP in the blood, which increases in response to inflammation, in some embodiments, it is used to detect inflammation and monitor conditions like infections and autoimmune diseases,
Cr Clearance (Creatinine Clearance): measures the rate at which creatinine is cleared from the blood by the kidneys; in some embodiments, it assesses kidney function,
Creatine Kinase (CK): an enzyme found in the heart, brain, and skeletal muscle; in some embodiments, elevated levels can indicate muscle damage, heart attack, or other conditions affecting muscle tissue,
Creatinine: measures the level of creatinine in the blood, which is a waste product of muscle metabolism, in some embodiments, it is used to assess kidney function,
Creatinine, Ur Random-Quest: measures the level of creatinine in a random urine sample, used to assess kidney function in some embodiments,
CRMP-5-IgG-Mayo (Collapsin Response-Mediator Protein-5 IgG): tests for antibodies against CRMP-5, associated with paraneoplastic neurological syndromes and certain types of cancer in some embodiments,
CTL CD34% (Cytotoxic T-Lymphocyte CD34 Percentage): measures the percentage of CD34+ cells among cytotoxic T-lymphocytes, used in the context of hematopoietic stem cell transplantation and other conditions in some embodiments,
CTL CD34 Abs (Cytotoxic T-Lymphocyte CD34 Absolute Count): measures the absolute count of CD34+ cells among cytotoxic T-lymphocytes, used similarly to the CD34 percentage in some embodiments,
CYP2C19: tests for genetic variations in the CYP2C19 enzyme, which affects the metabolism of certain drugs, in some embodiments it helps guide medication dosing and selection,
D-Dimer: measures the level of D-dimer in the blood, which is a fibrin degradation product; elevated levels can indicate blood clot formation and breakdown, used to diagnose conditions like deep vein thrombosis and pulmonary embolism in some embodiments,
Desethylamiodarone: measures the level of desethylamiodarone, a metabolite of the drug amiodarone, used to monitor therapy for arrhythmias in some embodiments,
Diff Comment (Differential Comment): provides additional interpretation or comments on the results of a white blood cell differential count, which categorizes the different types of white blood cells in some embodiments,
Differential?: refers to a white blood cell differential count, which categorizes the different types of white blood cells to diagnose and monitor infections, inflammation, and hematologic disorders in some embodiments,
Digoxin Level: measures the level of digoxin in the blood, used to monitor therapy for heart conditions like atrial fibrillation and heart failure; it ensures the level is therapeutic and not toxic in some embodiments,
Dohle Bodies: inclusions within white blood cells (neutrophils) that indicate infection, inflammation, or other stressors affecting the bone marrow,
Drug Screen performed at: indicates the location where a drug screening test was performed, which can detect the presence of various drugs and their metabolites in the body in some embodiments,
DVV Screen (Dilute Russell's Viper Venom Screen): a blood test used to detect lupus anticoagulant, an antibody associated with an increased risk of blood clots in some embodiments,
eAG (Estimated Average Glucose): provides an estimate of average blood glucose levels over the past two to three months, often derived from HbA1c results in some embodiments,
EBV DNA Quant Log-Quest (Epstein-Barr Virus DNA Quantification, Log): measures the amount of Epstein-Barr virus DNA in the blood, reported on a logarithmic scale, it is used to monitor EBV infections in some embodiments,
EBV DNA Quant-Quest (Epstein-Barr Virus DNA Quantification): measures the amount of Epstein-Barr virus DNA in the blood, used to diagnose and monitor EBV infections in some embodiments,
EBV IgG Capsid (Epstein-Barr Virus IgG Capsid Antigen): tests for IgG antibodies against the capsid protein of EBV, indicating past infection or reactivation in some embodiments,
EBV IgM Capsid (Epstein-Barr Virus IgM Capsid Antigen): tests for IgM antibodies against the capsid protein of EBV, indicating a recent or current infection in some embodiments,
EBV Quant Blood (Epstein-Barr Virus Quantitative Blood Test): measures the amount of Epstein-Barr virus in the blood, used to diagnose and monitor EBV infections in some embodiments,
EH HBS Ag (Hepatitis B Surface Antigen): tests for the presence of the hepatitis B surface antigen, indicating an active hepatitis B infection in some embodiments,
EH Hep C Ab (Hepatitis C Antibody): tests for antibodies against the hepatitis C virus, indicating exposure to the virus in some embodiments,
EH HIV 1/2 Ag/Ab (HIV 1/2 Antigen/Antibody Combination Test): tests for HIV-1 and HIV-2 antibodies and the p24 antigen of HIV-1, used to diagnose HIV infection in some embodiments,
Eosinophils: measures the number of eosinophils in the blood, a type of white blood cell involved in allergic reactions and parasitic infections in some embodiments, Erythropoietin (EPO): measures the level of erythropoietin in the blood, a hormone that stimulates red blood cell production, it is used to evaluate anemia and polycythemia in some embodiments,
ESR (Erythrocyte Sedimentation Rate): measures how quickly red blood cells settle at the bottom of a test tube, used to detect inflammation in some embodiments,
Estriol-OB: measures the level of estriol, an estrogen produced during pregnancy, used to monitor fetal well-being and placental function in some embodiments,
Ethanol Level: measures the amount of ethanol (alcohol) in the blood, used to assess alcohol intoxication in some embodiments,
Fe (Iron): measures the level of iron in the blood, used to diagnose and monitor conditions like anemia and hemochromatosis in some embodiments,
Ferritin Serum: measures the level of ferritin, a protein that stores iron, in the blood; it reflects the body's iron stores, in some embodiments,
Fibrillarin (U3 RNP): tests for antibodies against fibrillarin, a component of the U3 ribonucleoprotein complex, associated with certain autoimmune diseases like systemic sclerosis, in some embodiments,
Fibrinogen: measures the level of fibrinogen in the blood, a protein essential for blood clotting, abnormal levels can indicate bleeding disorders or thrombotic conditions in some embodiments,
FIO2 ABG (Fraction of Inspired Oxygen, Arterial Blood Gas): indicates the percentage of oxygen in the air mixture that a patient breathes, measured during arterial blood gas testing in some embodiments,
FIO2 CVL (Fraction of Inspired Oxygen, Central Venous Line): indicates the percentage of oxygen in the air mixture that a patient breathes, measured during central venous line monitoring in some embodiments,
FIO2 ECMO Post ABG (Fraction of Inspired Oxygen, Extracorporeal Membrane Oxygenation Post Arterial Blood Gas): indicates the percentage of oxygen in the air mixture that a patient breathes, measured after ECMO treatment, in some embodiments,
FIO2 ECMO PRE ABG (Fraction of Inspired Oxygen, Extracorporeal Membrane Oxygenation Pre Arterial Blood Gas): indicates the percentage of oxygen in the air mixture that a patient breathes, measured before ECMO treatment in some embodiments,
FIO2 VBG (Fraction of Inspired Oxygen, Venous Blood Gas): indicates the percentage of oxygen in the air mixture that a patient breathes, measured during venous blood gas testing, in some embodiments,
FIO2 MV (Fraction of Inspired Oxygen, Mechanical Ventilation): indicates the percentage of oxygen in the air mixture that a patient breathes, measured during mechanical ventilation in some embodiments,
Flow Differential-Quest: refers to flow cytometry testing, which differentiates and counts various cell types in a blood or bone marrow sample in some embodiments,
Folate: measures the level of folate (vitamin B9) in the blood, important for cell division and the production of DNA; low levels can cause anemia in some embodiments,
Free Kappa, Random Urine: measures the level of free kappa light chains in a random urine sample, in some embodiments used to diagnose and monitor plasma cell disorders like multiple myeloma,
Free K/L Ratio, Random Urine (Free Kappa/Lambda Ratio): measures the ratio of free kappa to free lambda light chains in a random urine sample, in some embodiments used to diagnose and monitor plasma cell disorders,
Free Lambda, Random Urine: measures the level of free lambda light chains in a random urine sample, in some embodiments used to diagnose and monitor plasma cell disorders like multiple myeloma,
Random Urine: refers to a urine sample collected at any time without special preparation, in some embodiments used for various tests like glucose, protein, or creatinine,
Free T3 (Free Triiodothyronine): measures the level of free T3 hormone in the blood, which is not bound to proteins and is biologically active, in some embodiments used to assess thyroid function,
Free T4 (Free Thyroxine): measures the level of free T4 hormone in the blood, in some embodiments used to assess thyroid function,
Free Testost % (Free Testosterone Percentage): measures the percentage of testosterone that is free (not bound to proteins) in the blood, in some embodiments used to diagnose hormonal disorders,
Free Testost Calc (Calculated Free Testosterone): provides a calculated value of free testosterone based on total testosterone, SHBG, and albumin levels,
FSH (Follicle-Stimulating Hormone): measures the level of FSH in the blood, which is involved in reproductive processes and in some embodiments helps assess fertility and gonadal function,
G6PD (Glucose-6-Phosphate Dehydrogenase): measures the activity of the G6PD enzyme in red blood cells, in some embodiments deficiency can lead to hemolytic anemia,
Gamma: refers to gamma-globulins, a group of proteins in the blood, which includes antibodies,
Gent Peak (Gentamicin Peak Level): measures the highest concentration of gentamicin in the blood after a dose, in some embodiments used to monitor therapeutic levels and avoid toxicity,
Gent Random (Gentamicin Random Level): measures the concentration of gentamicin in the blood at a random time, in some embodiments used for therapeutic drug monitoring,
Gent Trough (Gentamicin Trough Level): measures the lowest concentration of gentamicin in the blood before the next dose, in some embodiments used to monitor therapeutic levels and avoid toxicity,
GFR Calc (Calculated Glomerular Filtration Rate): estimates kidney function by calculating the rate at which blood is filtered through the kidneys,
GGT (Gamma-Glutamyl Transferase): measures the level of GGT in the blood, an enzyme that in some embodiments indicates liver and bile duct health,
GGT-Quest: Same as GGT, but performed by Quest Diagnostics,
GI 19-9 (CA 19-9): measures the level of CA 19-9, a tumor marker for pancreatic cancer and other gastrointestinal cancers,
Giant Platelets: refers to abnormally large platelets, often associated with certain hematologic disorders,
Glu ABG (Glucose Arterial Blood Gas): measures the glucose level in arterial blood gas samples,
Glucose Level: measures the level of glucose in the blood, in some embodiments used to diagnose and monitor diabetes,
Glu MV (Glucose Mechanical Ventilation): measures glucose levels in patients on mechanical ventilation,
Glu VBG (Glucose Venous Blood Gas): measures the glucose level in venous blood gas samples,
Granulocytes-Quest: measures the number of granulocytes (a type of white blood cell) in the blood, performed by Quest Diagnostics,
HA IgM (Hepatitis A IgM Antibody): tests for IgM antibodies against hepatitis A virus, indicating recent or current infection,
Haptoglobin: measures the level of haptoglobin in the blood, a protein that binds free hemoglobin, in some embodiments low levels can indicate hemolysis,
Haptoglobin-Quest: same as haptoglobin, but performed by Quest Diagnostics,
HBcIgM (Hepatitis B Core IgM Antibody): tests for IgM antibodies against hepatitis B core antigen, indicating recent or acute infection,
HB Core Ab-Total (Hepatitis B Core Antibody Total): tests for total antibodies (IgM and IgG) against hepatitis B core antigen, indicating past or current infection,
HBe Ab (Hepatitis B e Antibody): tests for antibodies against hepatitis B e antigen, indicating reduced infectivity,
HBe Ag (Hepatitis B e Antigen): tests for the presence of hepatitis B e antigen, indicating active viral replication and high infectivity,
HBS Ab (Hepatitis B Surface Antibody): tests for antibodies against hepatitis B surface antigen, in some embodiments indicating immunity to hepatitis B either through vaccination or recovery from infection,
HBS Ab Interp (Hepatitis B Surface Antibody Interpretation): provides interpretation of hepatitis B surface antibody test results,
HBs Ag-OB (Hepatitis B Surface Antigen-Obstetric): tests for hepatitis B surface antigen in pregnant women to assess the risk of transmitting the virus to the newborn,
HBVQNTX (Hepatitis B Virus Quantitative PCR): measures the amount of hepatitis B virus DNA in the blood, in some embodiments used to monitor viral load,
HCG Quant (Quantitative Human Chorionic Gonadotropin): measures the level of HCG in the blood, in some embodiments used to confirm and monitor pregnancy,
HCG Total-OB (Total Human Chorionic Gonadotropin-Obstetric): measures the total level of HCG in pregnant women, in some embodiments used to monitor pregnancy progression and diagnose conditions like ectopic pregnancy,
HCO3 ABG (Bicarbonate Arterial Blood Gas): measures the level of bicarbonate in arterial blood gas samples, in some embodiments used to assess acid-base balance,
HCO3 CVL (Bicarbonate Central Venous Line): measures the level of bicarbonate in blood samples from a central venous line,
HCO3 ECMO Post ABG (Bicarbonate Extracorporeal Membrane Oxygenation Post Arterial Blood Gas): measures the level of bicarbonate in arterial blood gas samples after ECMO treatment,
HCO3 ECMO PRE ABG (Bicarbonate Extracorporeal Membrane Oxygenation Pre Arterial Blood Gas): measures the level of bicarbonate in arterial blood gas samples before ECMO treatment,
HCO3 MV (Bicarbonate Mechanical Ventilation): measures the level of bicarbonate in blood samples from patients on mechanical ventilation,
HCO3 VBG (Bicarbonate Venous Blood Gas): measures the level of bicarbonate in venous blood gas samples,
Hct (Hematocrit): measures the percentage of red blood cells in the blood, in some embodiments used to diagnose and monitor anemia and other blood disorders,
Hct Performed at: indicates the location where the hematocrit test was performed,
HCV Genotype Y/N (Hepatitis C Virus Genotype Yes/No): tests for the presence of different genotypes of the hepatitis C virus,
HCV GEN PCR (Hepatitis C Virus Genotype PCR): uses PCR to determine the genotype of the hepatitis C virus,
HCVQNTX (Hepatitis C Virus Quantitative PCR): measures the amount of hepatitis C virus RNA in the blood, used to monitor viral load,
HDL (High-Density Lipoprotein): measures the level of HDL cholesterol in the blood, often referred to as “good” cholesterol; in some embodiments high levels are associated with a lower risk of cardiovascular disease,
Hmatocrit ECMO Post ABG: measures the percentage of red blood cells in arterial blood gas samples after ECMO treatment,
Hematocrit ECMO PRE ABG: measures the percentage of red blood cells in arterial blood gas samples before ECMO treatment,
Hemochron ACTHR (Activated Clotting Time High-Risk): measures the time it takes for blood to clot, e.g., for high-risk situations like cardiac surgery or anticoagulant therapy,
Hemochron ACTLR (Activated Clotting Time Low-Risk): measures the time it takes for blood to clot, in some embodiments used in low-risk situations or routine monitoring,
Hemoglobin ECMO Post ABG (Hemoglobin Extracorporeal Membrane Oxygenation Post Arterial Blood Gas): measures the level of hemoglobin in arterial blood samples after ECMO treatment,
Hemoglobin ECMO PRE ABG (Hemoglobin Extracorporeal Membrane Oxygenation Pre Arterial Blood Gas): measures the level of hemoglobin in arterial blood samples before ECMO treatment,
Hemoglobin Plasma: measures the level of hemoglobin in the plasma (liquid portion of blood),
Hep A IgG (Hepatitis A IgG Antibody): tests for IgG antibodies against hepatitis A virus, in some embodiments indicating past infection or immunity,
Hep B Surface Ag (Hepatitis B Surface Antigen): tests for the presence of the hepatitis B surface antigen, in some embodiments indicating active hepatitis B infection,
HepC Ab (Hepatitis C Antibody): tests for antibodies against hepatitis C virus, in some embodiments indicating exposure to the virus,
Hgb (Hemoglobin): measures the level of hemoglobin in the blood, in some embodiments used to diagnose and monitor anemia and other blood disorders,
Hgb A (Hemoglobin A): measures the level of hemoglobin A, in some embodiments used to assess overall hemoglobin levels,
HGB A1C (Hemoglobin A1c): measures the average blood glucose levels over the past two to three months, in some embodiments used to monitor diabetes management,
HGB-A1C (Hemoglobin A1c): same as HGB A1C, measures average blood glucose levels over the past two to three months,
Hgb A2 (Hemoglobin A2): measures the level of hemoglobin A2, which is elevated in some types of anemia like thalassemia,
Hgb ABG (Hemoglobin Arterial Blood Gas): measures the level of hemoglobin in arterial blood gas samples, e.g., in critical care settings,
Hgb Elect Interp (Hemoglobin Electrophoresis Interpretation): provides interpretation of hemoglobin electrophoresis results, in some embodiments used to diagnose and monitor hemoglobin disorders,
Hgb MV (Hemoglobin Mechanical Ventilation): measures hemoglobin levels in patients on mechanical ventilation,
Hgb Performed at: indicates the location where the hemoglobin test was performed,
HIAA-5 Ur 24 Hr (5-Hydroxyindoleacetic Acid 24-Hour Urine): measures the level of 5-HIAA in a 24-hour urine sample, in some embodiments used to diagnose and monitor neuroendocrine tumors like carcinoid syndrome,
HIAA-5 Ur Random (5-Hydroxyindoleacetic Acid Random Urine): measures the level of 5-HIAA in a random urine sample,
Histone Ab (Histone Antibodies): tests for antibodies against histones, which are proteins associated with DNA; in some embodiments positive results can indicate systemic lupus erythematosus (SLE) or other autoimmune disorders,
Histoplasma Antigen: measures the presence of Histoplasma antigens in the blood or urine, in some embodiments used to diagnose histoplasmosis, a fungal infection,
Histoplasma Antigen Interp (Histoplasma Antigen Interpretation): provides interpretation of the histoplasma antigen test results,
HIT AB (Heparin-Induced Thrombocytopenia Antibody): tests for antibodies associated with heparin-induced thrombocytopenia (HIT), a reaction to heparin that causes a drop in platelet count,
HIV 1/2 Ag/Ab (HIV 1/2 Antigen/Antibody Combination Test): tests for both HIV-1 and HIV-2 antibodies and the p24 antigen of HIV-1, used to diagnose HIV infection,
HIVIQNTX (HIV-1 Quantitative PCR): measures the amount of HIV-1 RNA in the blood, used to monitor viral load in HIV-infected individuals,
HIV Ab/Ag EIA-OB (HIV Antibody/Antigen Enzyme Immunoassay-Obstetric): tests for HIV antibodies and antigens in pregnant women to assess the risk of mother-to-child transmission,
HIV Confirm: refers to confirmatory testing for HIV following initial positive screening tests, typically involving more specific tests,
H-Jolly Bodies: abnormal red blood cell inclusions that indicate impaired spleen function or certain types of anemia,
HLA Ab for Post-Transplant C1q DSA (Human Leukocyte Antigen Antibodies for Post-Transplant C1q Donor-Specific Antibodies): tests for C1q donor-specific antibodies in transplant recipients, which in some embodiments can indicate rejection of the transplanted organ,
HLA Ab for Post-Transplant DSA (Human Leukocyte Antigen Antibodies for Post-Transplant Donor-Specific Antibodies): tests for donor-specific antibodies in transplant recipients, in some embodiments used to monitor organ rejection,
HLA Ab for Pre-Transplant PRA (Human Leukocyte Antigen Antibodies for Pre-Transplant Panel Reactive Antibody): measures the level of antibodies against various HLA antigens in potential organ transplant recipients, in some embodiments used to assess the likelihood of an immune response against a donor organ,
HLA Ab for Pre-Transplant SAB (Human Leukocyte Antigen Antibodies for Pre-Transplant Single Antigen Bead): measures specific antibodies against single HLA antigens in potential transplant recipients, in some embodiments used for detailed antigen matching before transplantation,
HLA-B 5701: tests for the presence of the HLA-B*5701 allele, which can be used in some embodiments to identify patients at risk of hypersensitivity reactions to the HIV medication abacavir,
HLA Class I/II Specimen: refers to the collection of samples for testing HLA Class I and Class II antigens, which are used for tissue matching in organ transplantation in some embodiments,
HLA-Lum SAB I/II DSA (Human Leukocyte Antigen Luminescent Single Antigen Bead Donor-Specific Antibodies): tests for donor-specific antibodies using luminescent single antigen bead technology, in some embodiments used in monitoring transplant recipients for rejection,
HLA-Lum SAB I/II MTH (Human Leukocyte Antigen Luminescent Single Antigen Bead Mismatch): identifies mismatches between donor and recipient HLA antigens using luminescent single antigen bead technology,
HLA Patient Type/Analysis Reason: refers to the type of HLA testing performed and the reason for the analysis, such as organ transplantation or autoimmune disease diagnosis,
Homocysteine: measures the level of homocysteine in the blood, an amino acid associated with cardiovascular risk and certain genetic disorders in some embodiments,
Hrs Col U Alb (Hours Collection Urine Albumin): measures albumin levels in a urine sample collected over a specific number of hours, in some embodiments used to assess kidney function,
Hrs Col U Ca (Hours Collection Urine Calcium): measures calcium levels in a urine sample collected over a specific number of hours, in some embodiments used to assess calcium metabolism and kidney function,
Hrs Col U Creat (Hours Collection Urine Creatinine): measures creatinine levels in a urine sample collected over a specific number of hours, in some embodiments used to assess kidney function,
Hrs Col U PEP (Hours Collection Urine Protein Electrophoresis): measures protein levels and types in a urine sample collected over a specific number of hours, in some embodiments used to diagnose and monitor kidney diseases,
Hrs Col U Prot (Hours Collection Urine Protein): measures total protein levels in a urine sample collected over a specific number of hours, in some embodiments used to assess kidney function and detect proteinuria,
Hrs Col U Urea (Hours Collection Urine Urea): measures urea levels in a urine sample collected over a specific number of hours, in some embodiments used to assess kidney function and protein metabolism,
HSCRP (High-Sensitivity C-Reactive Protein): measures low levels of C-reactive protein in the blood to, in some embodiments, assess inflammation and cardiovascular risk,
hs Troponin-I (High-Sensitivity Troponin I): measures low levels of troponin I, a protein released when the heart muscle is damaged, in some embodiments used to diagnose and assess heart attacks,
HSV 1 IgM Screen (Herpes Simplex Virus Type 1 IgM Antibody Screen): tests for IgM antibodies against HSV-1, indicating a recent or current infection with the herpes simplex virus type 1,
HSV 1 IgM Titer-Quest (Herpes Simplex Virus Type 1 IgM Antibody Titer): measures the concentration of IgM antibodies against HSV-1, used to assess the level of recent or active infection in some embodiments,
HSV 2 IgM Screen (Herpes Simplex Virus Type 2 IgM Antibody Screen): tests for IgM antibodies against HSV-2, in some embodiments indicating a recent or current infection with the herpes simplex virus type 2,
HSV 2 IgM Titer-Quest (Herpes Simplex Virus Type 2 IgM Antibody Titer): measures the concentration of IgM antibodies against HSV-2, in some embodiments used to assess the level of recent or active infection,
HT (Hemoglobin Test): Measures the level of hemoglobin, similar to Hgb,
Hypochrom (Hypochromasia): refers to red blood cells that are paler than normal, typically associated with certain types of anemia,
I-AGAP (Ionized Anion Gap): measures the anion gap in blood, in some embodiments used to evaluate metabolic acidosis and other electrolyte imbalances,
I-BE (Ionized Bicarbonate Equivalent): measures the level of bicarbonate in blood, which is part of the acid-base balance assessment,
I-BMP w/Ion Ca Performed at (Ionized Calcium Basic Metabolic Panel): refers to a Basic Metabolic Panel (BMP) that includes measurements of ionized calcium, in some embodiments used to assess kidney function, blood glucose levels, and electrolyte balance,
I-BUN (Ionized Blood Urea Nitrogen): measures the level of blood urea nitrogen (BUN) in the blood, in some embodiments used to assess kidney function and protein metabolism,
I-Ca (Ionized Calcium): measures the level of free calcium in the blood, which is involved in various bodily functions including bone health and muscle contraction,
I-Ca CRRT (Ionized Calcium Continuous Renal Replacement Therapy): measures ionized calcium levels in patients undergoing continuous renal replacement therapy (CRRT),
IC CD4 Absolute (Immunohematology CD4 Absolute): measures the absolute number of CD4 T cells in the blood, in some embodiments used to monitor immune system status, particularly in HIV-infected individuals,
IC CD4 Percent (Immunohematology CD4 Percent): measures the percentage of CD4 T cells in the blood relative to total lymphocytes, in some embodiments used to assess immune function,
I-Cl (Ionized Chloride): measures the level of chloride ions in the blood, in some embodiments used to evaluate electrolyte balance and acid-base status,
IC Lymph Absolute (Immunohematology Lymphocyte Absolute): measures the absolute number of lymphocytes in the blood, in some embodiments used to evaluate immune function,
IC Lymph Percent (Immunohematology Lymphocyte Percent): measures the percentage of lymphocytes in the blood relative to total white blood cells, in some embodiments used to assess immune function,
I-Creat (Ionized Creatinine): measures the level of creatinine in the blood, in some embodiments used to assess kidney function,
I-Creat Performed at (Ionized Creatinine Performed at): indicates the location where the ionized creatinine test was performed,
IC WBC (Immunohematology White Blood Cell Count): measures the total number of white blood cells in the blood, in some embodiments used to assess immune response and detect infections,
I-FlO2 (Ionized Fraction of Inspired Oxygen): measures the fraction of inspired oxygen in a patient's blood, in some embodiments used in critical care settings to monitor oxygenation,
IgA Immunoglobulin: measures the level of IgA, an antibody involved in mucosal immunity,
IgE (Immunoglobulin E): Measures the level of IgE, an antibody associated with allergic reactions and asthma,
IGF-1 (Insulin-like Growth Factor 1): measures the level of IGF-1, a hormone involved in growth and development, in some embodiments used to assess growth hormone activity,
IgG 1, IgG 2, IgG 3, IgG 4: measure the levels of different subclasses of IgG antibodies, which can provide information about immune function and certain immune disorders,
IgG Card (IgG Card Test): a rapid test for IgG antibodies, related to a specific infection or condition,
IgG (Immunoglobulin G): measures the level of IgG, the most abundant antibody in the blood, important for long-term immunity and protection against infections,
IgG CSF-Quest (IgG in Cerebrospinal Fluid-Quest): measures the level of IgG in cerebrospinal fluid, used to assess central nervous system disorders,
IgG Immunoglobulin: general measure of IgG levels in the blood,
IgG Index (IgG Index): a calculated ratio used to assess IgG levels in cerebrospinal fluid compared to serum, helpful in diagnosing multiple sclerosis and other neurological conditions,
IgG Total: measures the total level of IgG antibodies in the blood,
I-Glu (Ionized Glucose): measures the level of glucose in the blood, though typically glucose is measured as a whole rather than ionized,
IgM Card (IgM Card Test): a rapid test for IgM antibodies, related to a specific infection or condition,
IgM Immunoglobulin: measures the level of IgM, the first antibody produced in response to an infection,
IgM Varicella: measures IgM antibodies against varicella zoster virus, indicating a recent or current varicella (chickenpox) infection,
I-HCO3 (Ionized Bicarbonate): measures the level of bicarbonate in the blood, used to assess acid-base balance,
I-HCO3 cArt (Ionized Bicarbonate Central Arterial): measures ionized bicarbonate in central arterial blood, used in critical care settings,
I-HCO3 cVen (Ionized Bicarbonate Central Venous): measures ionized bicarbonate in central venous blood, in some embodiments used in critical care settings,
I-Hct (Ionized Hematocrit): measures the percentage of red blood cells in the blood,
I-Hgb (Ionized Hemoglobin): measures the level of hemoglobin in the blood,
I-K (Ionized Potassium): measures the level of potassium ions in the blood, involved in heart and muscle function,
I-Lactic (Ionized Lactic Acid): measures the level of lactic acid in the blood, in some embodiments used to assess tissue oxygenation and metabolic conditions,
Immuno G (Immunoglobulin G): testing for IgG antibodies, similar to other IgG tests mentioned above,
Serum-Quest: rfers to various serum tests performed by Quest Diagnostics,
I-Na (Ionized Sodium): measures the level of sodium ions in the blood, involved in maintaining fluid balance and proper nerve and muscle function,
Influenza A: tests for the presence of Influenza A virus, which causes the flu,
Influenza-A: similar to Influenza A, detects the influenza A virus,
Influenza B: tests for the presence of Influenza B virus, another strain of the flu virus,
Influenza-B: similar to Influenza B, detects the influenza B virus,
Inform Glu: refers to a glucose test, for informing or managing glucose levels,
INR (International Normalized Ratio): measures the time it takes for blood to clot, used to monitor anticoagulant therapy,
Insulin: measures the level of insulin in the blood, used to assess insulin production and regulation, particularly in diabetes,
Interleukin 2: measures the level of Interleukin 2, a cytokine involved in immune response,
Interp (1-3) Beta-D-Glucan: measures Beta-D-Glucan, a marker for fungal infections,
I-O2 Sat (Ionized Oxygen Saturation): measures the oxygen saturation level in the blood, important for assessing respiratory function,
Ion Ca (Ionized Calcium): measures the level of free calcium in the blood, important for various bodily functions,
Ion Ca CRRT (Ionized Calcium Continuous Renal Replacement Therapy): measures ionized calcium levels in patients undergoing continuous renal replacement therapy (CRRT),
I-Patient Position: refers to the position of the patient during blood sample collection, which can affect test results,
I-PCO2 (Ionized Partial Pressure of Carbon Dioxide): measures the partial pressure of carbon dioxide in the blood, important for assessing respiratory function,
I-PCO2 cArt (Ionized Partial Pressure of Carbon Dioxide Central Arterial): measures partial pressure of carbon dioxide in central arterial blood, used in critical care,
I-PCO2 cVen (Ionized Partial Pressure of Carbon Dioxide Central Venous): measures partial pressure of carbon dioxide in central venous blood, in some embodiments used in critical care,
I-P/F Ratio (Ionized PaO2/FiO2 Ratio): measures the ratio of partial pressure of oxygen to the fraction of inspired oxygen, in some embodiments used to assess lung function,
I-PH (Ionized pH): measures the pH level in the blood, in some embodiments for assessing acid-base balance,
I-PH cArt (Ionized pH Central Arterial): measures the pH in central arterial blood, in some embodiments used in critical care settings,
I-PH c Ven (Ionized pH Central Venous): measures the pH in central venous blood, in some embodiments used in critical care settings,
I-PO2 (Ionized Partial Pressure of Oxygen): measures the partial pressure of oxygen in the blood, in some embodiments used to assess oxygenation,
I-PO2 Art (Ionized Partial Pressure of Oxygen Arterial): measures partial pressure of oxygen in arterial blood, in some embodiments used in critical care,
I-PO2 Ven (Ionized Partial Pressure of Oxygen Venous): measures partial pressure of oxygen in venous blood, in some embodiments used in critical care,
I-Sample: refers to the specific sample being tested,
I-TCO2 (Ionized Total Carbon Dioxide): measures the total amount of carbon dioxide in the blood, in some embodiments used to assess acid-base balance,
I-Temp Pat (Ionized Temperature Patient): refers to patient temperature,
JC Virus (John Cunningham Virus): tests for the presence of JC virus, which can cause progressive multifocal leukoencephalopathy (PML) in immunocompromised individuals,
CSF (Cerebrospinal Fluid): tests performed on cerebrospinal fluid to diagnose neurological disorders,
Jo-1 Antibody: measures antibodies against Jo-1, which are associated with certain types of inflammatory myopathies,
Kappa FLC (Kappa Free Light Chains): measures kappa free light chains in the blood, used to diagnose and monitor multiple myeloma and other plasma cell disorders,
K VBG (Potassium Venous Blood Gas): measures potassium levels in venous blood, used to assess electrolyte balance,
Lactic Acid: measures the level of lactic acid in the blood, which can indicate tissue hypoxia or metabolic conditions in some embodiments,
Lambda FLC (Lambda Free Light Chains): measures lambda free light chains in the blood, in some embodiments used to diagnose and monitor multiple myeloma and other plasma cell disorders,
LDH (Lactate Dehydrogenase): measures the level of LDH, an enzyme involved in energy production, often used to assess tissue damage and disease,
LDL (Low-Density Lipoprotein): measures the level of LDL cholesterol, often referred to as “bad” cholesterol, which can contribute to cardiovascular disease,
Lead: measures the level of lead in the blood, used to assess lead poisoning,
Leiden Factor V Analysis: tests for the presence of Factor V Leiden mutation, which increases the risk of blood clots in some embodiments,
Leptin-Quest: measures leptin levels, a hormone involved in regulating appetite and energy expenditure,
LGI1-IgG-Mayo (Leucine-rich Glioma Inactivated 1 Antibody): measures antibodies against LGI1, associated with certain autoimmune encephalitides,
LH (Luteinizing Hormone): measures the level of LH, a hormone involved in regulating the menstrual cycle and testosterone production,
Lidocaine: measures the level of lidocaine, a local anesthetic, in the blood,
Lipase: measures the level of lipase, an enzyme involved in fat digestion, used to diagnose pancreatic disorders,
Liver Kidney Ab-Quest: refers to antibodies related to liver and kidney diseases, tested by Quest Diagnostics,
LUP Interp (Lupus Anticoagulant Interpretation): [rovides interpretation for lupus anticoagulant tests, used to diagnose antiphospholipid syndrome,
Lupus Anticoagulant Reason For Exam: indicates the reason for testing for lupus anticoagulant, which is associated with an increased risk of blood clots,
Lyme Ab Screen: tests for antibodies against Borrelia burgdorferi, the bacterium that causes Lyme disease,
Lymphocytes: measures the number of lymphocytes, a type of white blood cell involved in immune response,
Macro: refers to testing for macrocytes, which are abnormally large red blood cells, indicating possible anemia,
Magnesium: measures the level of magnesium in the blood, important for muscle and nerve function,
Magnesium Performed at: indicates where the magnesium test was performed,
MAS Ferritin: measures ferritin levels in the context of macrophage activation syndrome (MAS), a serious complication of systemic diseases,
MCH (Mean Corpuscular Hemoglobin): measures the average amount of hemoglobin in red blood cells,
MCHC (Mean Corpuscular Hemoglobin Concentration): measures the average concentration of hemoglobin in red blood cells,
MCV (Mean Corpuscular Volume): measures the average size of red blood cells,
MDA-5 (P140) (CADM-140): tests for antibodies against MDA-5, associated with a type of autoimmune myopathy,
Meta: refers to a specific type of test or analysis related to “meta” as a prefix,
MetHB % ABG (Methemoglobin Percentage Arterial Blood Gas): measures the percentage of methemoglobin in the blood, used to assess oxygen-carrying capacity,
MetHb % VBG (Methemoglobin Percentage Venous Blood Gas): measures the percentage of methemoglobin in venous blood,
Methylmalonic Acid Lvl: measures the level of methylmalonic acid in the blood, in some embodiments used to diagnose vitamin B12 deficiency,
MI-2 (Myositis-specific Antibody 2): tests for MI-2 antibodies, which are associated with certain types of inflammatory myopathies,
Micro: measure the number of microcytes, which are abnormally small red blood cells, indicating possible anemia,
Monocytes: Measures the number of monocytes, a type of white blood cell involved in immune response and inflammation,
Monocytes-Quest: Measures monocyte levels, specifically tested by Quest Diagnostics,
MPO (Myeloperoxidase): measures the level of myeloperoxidase, an enzyme released by white blood cells during inflammation, in some embodiments used to assess conditions like vasculitis and myocardial infarction,
MPV (Mean Platelet Volume): measures the average size of platelets, which can indicate platelet production and activation,
M-spike 1: refers to a specific peak on a protein electrophoresis test, indicating the presence of a monoclonal protein, which can be associated with conditions like multiple myeloma,
M-spike 2: Similar to M-spike 1, another peak indicating a monoclonal protein,
Mycophenolic Acid: measures the level of mycophenolic acid, a drug used in immunosuppressive therapy, to ensure proper dosing and effectiveness in some embodiments,
Mycoplasma pneumoniae: tests for the presence of Mycoplasma pneumoniae bacteria, which causes respiratory infections,
Myelo (Myeloblasts): refers to measuring myeloblast levels, myeloblast are immature white blood cells, which can be elevated in conditions like leukemia,
Na ABG (Sodium Arterial Blood Gas): measures sodium levels in arterial blood, used to assess electrolyte balance and fluid status,
Na VBG (Sodium Venous Blood Gas): measures sodium levels in venous blood,
Neutrophils: measures the number of neutrophils, a type of white blood cell that helps fight infection,
NH4 (Ammonia): measures the level of ammonia in the blood, used to assess liver function and metabolic disorders,
Nicotinamide-Quest: measures nicotinamide levels, a form of vitamin B3, tested by Quest Diagnostics,
Nicotinic Acid-Quest: measures nicotinic acid levels, another form of vitamin B3, tested by Quest Diagnostics,
NRBC (Nucleated Red Blood Cells): measures the number of nucleated red blood cells, which are typically present in the bone marrow and can indicate conditions like anemia or fetal distress,
NRBC Inst (Nucleated Red Blood Cells Inst): refers to an instant or specific measurement of nucleated red blood cells,
NT-proBNP-Quest (N-Terminal Pro B-Type Natriuretic Peptide): measures levels of NT-proBNP, a marker used to assess heart failure, specifically tested by Quest Diagnostics,
N-Type-Mayo: refers to a specific test for N-type antibodies or markers, potentially related to neurological conditions, tested by Mayo Clinic,
NXP-2 (P140): tests for antibodies against NXP-2 (P140), associated with certain autoimmune diseases like dermatomyositis,
O2 Content ABG (Oxygen Content Arterial Blood Gas): measures the total amount of oxygen in arterial blood, including both bound and free oxygen,
O2 Content CVL (Oxygen Content Central Venous Line): measures oxygen content in blood from a central venous line,
O2 Content ECMO Post ABG (Oxygen Content ECMO Post Arterial Blood Gas): measures oxygen content in blood from an ECMO system post-arterial blood gas sampling,
O2 Content ECMO PRE ABG (Oxygen Content ECMO Pre-Arterial Blood Gas): measures oxygen content in blood from an ECMO system pre-arterial blood gas sampling,
O2 Content MV (Oxygen Content Mechanical Ventilation): measures oxygen content in blood from a mechanically ventilated patient,
O2 Content VBG (Oxygen Content Venous Blood Gas): measures the total oxygen content in venous blood,
O2Hb % ABG (Oxygenated Hemoglobin Percentage Arterial Blood Gas): measures the percentage of hemoglobin that is oxygenated in arterial blood,
O2Hb % CVL (Oxygenated Hemoglobin Percentage Central Venous Line): measures the percentage of oxygenated hemoglobin in blood from a central venous line,
O2Hb % ECMO Post ABG (Oxygenated Hemoglobin Percentage ECMO Post-Arterial Blood Gas): measures the percentage of oxygenated hemoglobin in blood from ECMO post-arterial blood gas sampling,
O2Hb % ECMO PRE ABG (Oxygenated Hemoglobin Percentage ECMO Pre-Arterial Blood Gas): measures the percentage of oxygenated hemoglobin in blood from ECMO pre-arterial blood gas sampling,
O2Hb % MV (Oxygenated Hemoglobin Percentage Mechanical Ventilation): measures the percentage of oxygenated hemoglobin in blood from a mechanically ventilated patient,
O2 Hb % VBG (Oxygenated Hemoglobin Percentage Venous Blood Gas): measures the percentage of oxygenated hemoglobin in venous blood,
O2 Sat ABG (Oxygen Saturation Arterial Blood Gas): measures the percentage of oxygen bound to hemoglobin in arterial blood,
O2 Sat CVL (Oxygen Saturation Central Venous Line): measures oxygen saturation in blood from a central venous line,
O2 Sat ECMO Post ABG (Oxygen Saturation ECMO Post-Arterial Blood Gas): measures oxygen saturation in blood from ECMO post-arterial blood gas sampling,
O2 Sat ECMO PRE ABG (Oxygen Saturation ECMO Pre-Arterial Blood Gas): measures oxygen saturation in blood from ECMO pre-arterial blood gas sampling,
O2 Sat MV (Oxygen Saturation Mechanical Ventilation): measures oxygen saturation in blood from a mechanically ventilated patient,
Occult Bld FIT (Fecal Immunochemical Test): tests for hidden (occult) blood in stool, myeloblast used to screen for colorectal cancer,
Osmo (Osmolality): measures the concentration of solutes in the blood, which in some embodiments can indicate hydration status and kidney function,
Other High Risk-HPV DNA: tests for the presence of high-risk human papillomavirus (HPV) DNA, which in some embodiments is associated with cervical cancer and other HPV-related cancers,
Ovalocytes: measures the presence of oval-shaped red blood cells, which in some embodiments can indicate conditions like anemia or liver disease,
PAN-SARS RNA: tests for the presence of SARS-COV RNA, in some embodiments used to detect SARS (Severe Acute Respiratory Syndrome) or similar coronavirus infections,
PAPP-A-OB (Pregnancy-Associated Plasma Protein A): measures the level of PAPP-A, a protein associated with pregnancy, in some embodiments used in prenatal screening for chromosomal abnormalities,
Path Hgb Electro Review: review of hemoglobin electrophoresis results by a pathologist to identify abnormal hemoglobin types,
Pathologist IFE Review: review of immunofixation electrophoresis (IFE) results by a pathologist, which is used to detect and analyze monoclonal proteins,
Pathologist SPE Review: review of serum protein electrophoresis (SPE) results by a pathologist to analyze protein levels and identify abnormal proteins,
Pathologist U IFE Random Review: review of random urine immunofixation electrophoresis results by a pathologist to analyze urine proteins,
Pathologist U PEP Random Review: review of random urine protein electrophoresis results by a pathologist,
Pathologist U PEP Timed Review: review of timed urine protein electrophoresis results by a pathologist, which measures protein levels in urine over a specified time period,
Pathologist Ur IFE Timed Review: review of timed urine immunofixation electrophoresis results by a pathologist,
Pat Temp ABG (Patient Temperature Arterial Blood Gas): measures patient temperature along with arterial blood gas levels,
Pat Temp CVL (Patient Temperature Central Venous Line): measures patient temperature using a central venous line,
Pat Temp ECMO Post ABG (Patient Temperature ECMO Post-Arterial Blood Gas): measures patient temperature along with arterial blood gas levels from ECMO post-sampling,
Pat Temp ECMO PRE ABG (Patient Temperature ECMO Pre-Arterial Blood Gas): measures patient temperature along with arterial blood gas levels from ECMO pre-sampling,
Pat Temp MV (Patient Temperature Mechanical Ventilation): measures patient temperature in a mechanically ventilated setting,
Pat Temp VBG (Patient Temperature Venous Blood Gas): measures patient temperature along with venous blood gas levels,
PCA-1-Mayo: Tests for PCA-1 antibodies, associated with autoimmune neurological disorders, specifically tested by Mayo Clinic,
PCA-2-Mayo: tests for PCA-2 antibodies, another marker associated with neurological autoimmune disorders, tested by Mayo Clinic,
PCA-Type Tr-Mayo: refers to a specific type of PCA (paraneoplastic syndrome-associated) antibody test, specifically tested by Mayo Clinic,
pCO2 ABG (Partial Pressure of Carbon Dioxide Arterial Blood Gas): measures the amount of carbon dioxide dissolved in arterial blood, in some embodiments used to assess respiratory function,
pCO2 CVL (Partial Pressure of Carbon Dioxide Central Venous Line): measures carbon dioxide levels in blood from a central venous line,
pCO2 ECMO Post ABG (Partial Pressure of Carbon Dioxide ECMO Post-Arterial Blood Gas): measures carbon dioxide levels in blood from ECMO post-arterial blood gas sampling,
pCO2 ECMO PRE ABG (Partial Pressure of Carbon Dioxide ECMO Pre-Arterial Blood Gas): measures carbon dioxide levels in blood from ECMO pre-arterial blood gas sampling,
pCO2 MV (Partial Pressure of Carbon Dioxide Mechanical Ventilation): measures carbon dioxide levels in blood from a mechanically ventilated patient,
PCO2 VBG (Partial Pressure of Carbon Dioxide Venous Blood Gas): measures carbon dioxide levels in venous blood,
P/F Ratio (PaO2/FiO2 Ratio): measures the ratio of arterial oxygen partial pressure (PaO2) to the fraction of inspired oxygen (FiO2), in some embodiments used to assess the severity of respiratory distress,
PGH A-Subunit-Mayo: tests for the presence of the A-subunit of placental growth hormone (PGH), specifically tested by Mayo Clinic,
pH BG (pH Blood Gas): measures the pH level in blood gas samples, indicating the acidity or alkalinity of the blood,
pH CVL (pH Central Venous Line): measures the pH level in blood from a central venous line,
pH ECMO Post ABG (pH ECMO Post-Arterial Blood Gas): measures the pH level in blood from ECMO post-arterial blood gas sampling,
pH ECMO PRE ABG (pH ECMO Pre-Arterial Blood Gas): measures the pH level in blood from ECMO pre-arterial blood gas sampling,
Phenytoin: measures the level of phenytoin, a medication used to control seizures, to ensure therapeutic levels,
pH MV (pH Mechanical Ventilation): measures the pH level of blood in a mechanically ventilated patient,
Phos (Phosphate): measures phosphate levels in the blood, important for bone health and energy storage,
Phosphatidylethanol (PEth): tests for PEth, a specific biomarker of alcohol consumption, indicating recent drinking,
pH VBG (pH Venous Blood Gas): measures the pH level in venous blood gas samples, reflecting the blood's acidity or alkalinity,
PL-12: tests for PL-12 antibodies, associated with certain autoimmune diseases like antisynthetase syndrome,
PL-7: tests for PL-7 antibodies, associated with antisynthetase syndrome and some forms of idiopathic inflammatory myopathy,
Platelet: measures the number of platelets in the blood, involved in blood clotting,
Platelet Function P2Y12: assesses platelet function, particularly in relation to the P2Y12 receptor, which is involved in platelet aggregation and clotting,
Plt Agg (Platelet Aggregation): measures how well platelets clump together, which is important for proper blood clotting,
pO2 ABG (Partial Pressure of Oxygen Arterial Blood Gas): measures the amount of oxygen dissolved in arterial blood, reflecting respiratory function,
pO2 CVL (Partial Pressure of Oxygen Central Venous Line): measures oxygen levels in blood from a central venous line,
pO2 ECMO Post ABG (Partial Pressure of Oxygen ECMO Post-Arterial Blood Gas): measures oxygen levels in blood from ECMO post-arterial blood gas sampling,
pO2 ECMO PRE ABG (Partial Pressure of Oxygen ECMO Pre-Arterial Blood Gas): measures oxygen levels in blood from ECMO pre-arterial blood gas sampling,
pO2 MV (Partial Pressure of Oxygen Mechanical Ventilation): measures oxygen levels in blood from a mechanically ventilated patient,
PO2 VBG (Partial Pressure of Oxygen Venous Blood Gas): measures oxygen levels in venous blood,
Polychrom (Polychromasia): measures the presence of polychromatic red blood cells, which can indicate conditions like anemia or recovery from blood loss,
Potassium: measures potassium levels in the blood, involved in cell function, muscle contraction, and heart function,
P/Q-Type-Mayo: tests for P/Q-type antibodies, often associated with certain types of autoimmune neurological conditions, tested by Mayo Clinic,
PR3 (Proteinase 3): measures levels of PR3 antibodies, which are associated with granulomatosis with polyangiitis (Wegener's granulomatosis),
Prealb (Prealbumin): measures prealbumin levels, a protein that reflects nutritional status and liver function,
Pregnant?: Aatest to determine pregnancy status, such as through hCG levels,
Procalcitonin: measures procalcitonin levels, a marker used in some embodiments to assess bacterial infection and sepsis,
Progressive Lyme: refers to tests related to Lyme disease, assessing progression or chronicity of the infection,
Prolactin Level: measures prolactin levels, a hormone involved in lactation and reproductive health,
Promyelo (Promyelocytes): measures the number of promyelocytes, immature white blood cells, which in some embodiments can be elevated in conditions like acute promyelocytic leukemia,
Protein: measures total protein levels in the blood, which in some embodiments can help assess liver function, kidney function, and nutritional status,
Prothrombin Gene Analysis-Quest: genetic test to assess for mutations in the prothrombin gene, which can increase the risk of blood clotting disorders, tested by Quest Diagnostics,
PSA Total (Prostate-Specific Antigen): measures PSA levels, a marker used in some embodiments to screen for prostate cancer and other prostate conditions,
PT (Prothrombin Time): measures the time it takes for blood to clot, in some embodiments involved in assessing bleeding disorders and the effectiveness of anticoagulant therapy,
PT Gene Interp-Quest: interpretation of prothrombin gene mutation analysis, tested by Quest Diagnostics,
PTH (Parathyroid Hormone): measures levels of parathyroid hormone, which regulates calcium and phosphorus levels in the blood,
PTH Peptide-Mayo: tests for PTH-related peptide levels, which can be associated with certain cancers and hypercalcemia, tested by Mayo Clinic,
PT Performed at: indicates the location where prothrombin time testing was performed,
PTT (Partial Thromboplastin Time): measures the time it takes for blood to clot, used to evaluate the intrinsic pathway of the coagulation cascade,
PTT-LA (Partial Thromboplastin Time-Lupus Anticoagulant): measures PTT with additional testing for lupus anticoagulant, an antibody that can increase clotting risk,
Random Urine Electrophoresis Consult: review of random urine electrophoresis results, which analyzes proteins in urine to detect abnormalities,
RBC (Red Blood Cells): measures the number of red blood cells in the blood, important for oxygen transport,
RBC Retic (Reticulocytes): measures the number of reticulocytes, immature red blood cells, which can indicate bone marrow activity and anemia,
RBCs-Quest: measures red blood cells, specifically tested by Quest Diagnostics,
RDW (Red Cell Distribution Width): measures the variation in size of red blood cells, which in some embodiments helps diagnose types of anemia and other conditions,
Retic Absolute (Absolute Reticulocyte Count): measures the number of reticulocytes (immature red blood cells) in the blood, in some embodiments indicating bone marrow activity,
Reticulocyte: measures the percentage or absolute count of reticulocytes, which in some embodiments can help assess bone marrow function and anemia,
RF Quant (Rheumatoid Factor Quantitative): measures the level of rheumatoid factor in the blood, in some embodiments used to diagnose and monitor rheumatoid arthritis and other autoimmune diseases,
RNA Polymerase III Ab-Quest: tests for antibodies against RNA polymerase III, associated with certain autoimmune conditions, specifically tested by Quest Diagnostics,
RPR (Rapid Plasma Reagin): a non-treponemal test for syphilis that detects antibodies produced in response to the infection,
RPR Titer: measures the concentration of antibodies detected by the RPR test, indicating the severity of syphilis infection,
Salicylate: measures the level of salicylate in the blood, used to monitor for toxicity or therapeutic levels of aspirin,
SARS-COV2 RNA: detects the presence of SARS-CoV-2 RNA, the virus responsible for COVID-19, typically through PCR testing,
% Sat (Saturation): measures the percentage of hemoglobin that is saturated with oxygen, in some embodiments for assessing respiratory function,
Schistocyte: measures the presence of schistocytes (fragmented red blood cells), which can indicate hemolytic anemia or other blood disorders,
SCL70: tests for anti-Scl-70 antibodies, associated with systemic sclerosis (scleroderma),
Selenium: measures selenium levels in the blood, an important trace element for antioxidant defense and thyroid function,
SER CRE (Serum Creatinine): measures creatinine levels in the blood, an indicator of kidney function,
Serotonin: measures serotonin levels in the blood, which can be related to mood disorders and other health conditions,
Serotonin Release Assay: assesses how serotonin is released from platelets, used in diagnosing certain types of serotonin-related conditions,
Serum Drug Confirmation: confirms the presence of specific drugs in the serum, in some embodiments used to verify initial drug screening results,
Serum Drug Screen: screens for the presence of various drugs in the serum, in some embodiments used for drug testing,
Serum Electrophoresis Consult: a consultation or review of serum electrophoresis results, which analyze proteins in the blood to detect abnormalities,
Sex Hormone Binding Glob (SHBG): measures levels of sex hormone-binding globulin, a protein that binds sex hormones, influencing their activity,
Sickle Cells: measures the presence of sickle-shaped red blood cells, which are characteristic of sickle cell anemia,
Siderocyte: measures the presence of siderocytes (red blood cells containing iron granules), which can indicate iron metabolism disorders,
Sirolimus: measures levels of sirolimus, an immunosuppressive drug used in some embodiments to prevent organ transplant rejection,
Sodium: measures sodium levels in the blood, involved in maintaining fluid balance and proper nerve and muscle function,
Source-Adenovirus Qnt: quantifies adenovirus in a sample, in some embodiments used to assess adenovirus infection,
Source Ag-Quest: refers to testing for specific antigens related to a pathogen or condition, performed by Quest Diagnostics,
Source EBV DNA-Quest: quantifies Epstein-Barr Virus (EBV) DNA, in some embodiments used to assess EBV infection, tested by Quest Diagnostics,
Spherocyte: measures the presence of spherocytes (spherical red blood cells), which in some embodiments can indicate hemolytic anemia,
S. pneumoniae Antigens: detects antigens from Streptococcus pneumoniae, a bacteria that can cause pneumonia and other infections,
Urine-Quest: refers to urine tests performed by Quest Diagnostics, which includes a range of tests from general urinalysis to specific biomarkers,
S Prot (Serum Protein): measures total serum protein levels, used to evaluate liver function, kidney function, and nutritional status,
SRA Confirm (Serotonin Release Assay Confirmation): confirms results from a serotonin release assay, used in diagnosing serotonin-related conditions,
SRP (Signal Recognition Particle): tests for antibodies against signal recognition particles, associated with certain autoimmune myopathies,
SSA (Anti-SSA/Ro): tests for antibodies against SSA/Ro antigens, associated with autoimmune conditions like Sjögren's syndrome and lupus,
SSB (Anti-SSB/La): tests for antibodies against SSB/La antigens, associated with autoimmune conditions like Sjögren's syndrome and lupus,
Stippled RBC (Stippled Red Blood Cells): measures the presence of stippled red blood cells, which can be seen in certain types of anemia and lead poisoning,
Stomato: refers to the presence of stomatocytes (abnormally shaped red blood cells) in the blood, which in some embodiments can indicate certain types of anemia or liver disease,
Strep Pneumo IgG Ab-23 serotypes: measures IgG antibodies against 23 serotypes of Streptococcus pneumoniae, in some embodiments used to assess immune response to pneumococcal vaccination or infection,
Striate Mscl Ab-Mayo: tests for antibodies against striated muscle, associated with certain autoimmune myopathies, tested by Mayo Clinic,
Syphilis EIA-OB (Enzyme Immunoassay): a test for syphilis using an enzyme immunoassay to detect antibodies related to syphilis infection,
T3 (Triiodothyronine): measures levels of T3, a thyroid hormone that regulates metabolism,
Reverse T3 (Reverse Triiodothyronine): measures levels of reverse T3, a metabolite of T3 that can be elevated in certain conditions affecting thyroid function,
T3 Total: measures the total amount of T3 hormone in the blood, including both bound and free T3,
T4 Total: measures the total amount of thyroxine (T4) in the blood, including both bound and free T4,
Tacrolimus: measures levels of tacrolimus, an immunosuppressive drug used to prevent organ transplant rejection,
Target Cell: measures the presence of target cells (red blood cells with a central area of hemoglobin surrounded by a ring of pallor), which in some embodiments can be seen in conditions like liver disease and thalassemia,
T Bili-Quest (Total Bilirubin): measures total bilirubin levels, in some embodiments used to evaluate liver function and assess jaundice,
TB Panel-A and TB Panel-B: tests for various markers related to tuberculosis infection, including tuberculin skin tests and interferon-gamma release assays,
Tear Cell: measures the presence of tear-shaped red blood cells, which in some embodiments can be seen in conditions like myelofibrosis,
Testost Total (Total Testosterone): measures total testosterone levels, involved in assessing hormonal status and conditions like hypogonadism in some embodiments,
Tg (Thyroglobulin): measures thyroglobulin levels, a protein produced by the thyroid gland, in some embodiments used to monitor thyroid cancer and other thyroid disorders,
TgAb (Thyroglobulin Antibodies): tests for antibodies against thyroglobulin, often elevated in autoimmune thyroid diseases,
Thiamine: measures thiamine (vitamin B1) levels, involved in neurological and metabolic health,
Thyro Ab (Thyroid Antibodies): tests for antibodies against thyroid tissue, in some embodiments used to diagnose autoimmune thyroid disorders,
Thyroglobulin: measures the levels of thyroglobulin, a thyroid-specific protein that can be used to monitor thyroid cancer treatment,
Thyro Recept Ab-Mayo: tests for antibodies against thyroid stimulating hormone (TSH) receptors, associated with Graves' disease, tested by Mayo Clinic
TIBC (Total Iron-Binding Capacity): measures the blood's ability to bind and transport iron, used to assess iron deficiency or overload,
TIF1 GAMMA (P155/140): tests for TIF1 gamma antibodies, associated with dermatomyositis and other autoimmune myopathies,
Timed Urine Electrophoresis Consult: review of timed urine electrophoresis results, which analyzes proteins in urine to detect abnormalities over a specific time period,
total protein: measures the total amount of protein in the blood, which in some embodiments helps assess liver and kidney function as well as nutritional status,
Tot Vol U Alb (Total Volume Urine Albumin): measures the total volume of albumin in urine, in some embodiments used to assess kidney function,
Tot Vol U Ca (Total Volume Urine Calcium): measures the total volume of calcium in urine, involved in evaluating calcium metabolism and kidney function in some embodiments,
Tot Vol U Creat (Total Volume Urine Creatinine): measures the total volume of creatinine in urine, in some embodiments used to evaluate kidney function and proteinuria,
Tot Vol U PEP (Total Volume Urine Protein Electrophoresis): measures the total volume of proteins in urine using electrophoresis, in some embodiments used to diagnose and monitor kidney conditions,
Tot Vol U Prot (Total Volume Urine Protein): measures the total volume of protein in urine, used to assess kidney function in some embodiments,
Tot Vol U Urea (Total Volume Urine Urea): measures the total volume of urea in urine, which can help assess kidney function and hydration status in some embodiments,
Toxic Gran (Toxic Granulation): measures the presence of toxic granulation in neutrophils, which can be a sign of infection or inflammation in some embodiments,
Toxo IgG: tests for IgG antibodies to Toxoplasma gondii, indicating past infection or exposure,
Toxo IgM: tests for IgM antibodies to Toxoplasma gondii, indicating recent or active infection in some embodiments,
Toxoplasma gondii IgG Ab: measures IgG antibodies to Toxoplasma gondii, confirming exposure or past infection in some embodiments,
CSF-Quest: refers to cerebrospinal fluid testing performed by Quest Diagnostics, used to diagnose neurological conditions,
TPMT Level (Thiopurine Methyltransferase): measures TPMT enzyme levels, which affect the metabolism of certain medications used in autoimmune conditions and cancer in some embodiments,
Transferrin: measures transferrin levels, a protein that binds and transports iron in the blood,
Treponema Ab: tests for antibodies against Treponema pallidum, the bacteria that causes syphilis,
Troponin T-Quest: measures levels of troponin T, a cardiac biomarker used to diagnose heart attacks, tested by Quest Diagnostics,
TSH-OB (Thyroid Stimulating Hormone): measures TSH levels, which regulate thyroid function and can indicate thyroid disorders,
TSH (Thyroid Stimulating Hormone): measures TSH levels, important for evaluating thyroid gland function,
TSI (Thyroid Stimulating Immunoglobulin): measures TSI levels, which are elevated in Graves' disease, a type of hyperthyroidism,
T-SPOT (TB): an interferon-gamma release assay (IGRA) test used to detect tuberculosis infection,
TTG (Tissue Transglutaminase): tests for antibodies against tissue transglutaminase, used to diagnose celiac disease,
Type 16-HPV DNA: tests for the presence of HPV type 16 DNA, a high-risk strain associated with cervical cancer and other HPV-related cancers,
Type 18-HPV DNA: tests for the presence of HPV type 18 DNA, another high-risk strain associated with cervical and other HPV-related cancers,
U2 snRNP: tests for antibodies against U2 small nuclear ribonucleoproteins, associated with certain autoimmune diseases like mixed connective tissue disease,
U Amorph: refers to amorphous deposits or crystals in urine, which can be a normal finding or indicate certain conditions,
U Ampheta: tests for the presence of amphetamines in urine, often used in drug screening,
UA Performed at: indicates that a urinalysis (UA) was performed, used to evaluate various aspects of urine composition and detect abnormalities,
U Bact: tests for the presence of bacteria in urine, in some embodiments used to diagnose urinary tract infections,
U Barb: tests for the presence of barbiturates in urine, used in drug screening in some embodiments,
U Benz: tests for benzodiazepines in urine, used in drug screening in some embodiments,
U Blood: tests for the presence of blood in urine, which can indicate bleeding or injury in the urinary tract in some embodiments,
U Buprenorphine: tests for buprenorphine, a medication used to treat opioid addiction, in urine,
U Cannabin: tests for cannabinoids (e.g., THC) in urine, used to detect marijuana use in some embodiments,
U CaOx Cry: measures calcium oxalate crystals in urine, which can be a sign of kidney stones or metabolic disorders in some embodiments,
UCG (Urine Chorionic Gonadotropin): measures levels of human chorionic gonadotropin (hCG) in urine, used for pregnancy testing in some embodiments,
UCG Performed at: indicates that a urine pregnancy test (UCG) was performed,
U C Gran: refers to granular casts in urine, which can be seen in various kidney conditions,
U Chlam RNA: tests for RNA of Chlamydia trachomatis, used to diagnose Chlamydia infections in some embodiments,
U Clarity: assesses the clarity of urine, which can indicate the presence of particles or abnormalities in some embodiments,
U Cl Random: measures chloride levels in random urine samples,
U Cocain Meta: tests for cocaine metabolites in urine, used in drug screening in some embodiments,
U Color: measures the color of urine, which can provide information about hydration status and other factors in some embodiments,
U Creat R (Creatinine Random): measures creatinine levels in random urine samples, used to assess kidney function, in some embodiments,
U Eos Stain: refers to eosin staining of urine sediment, which can help identify certain types of cells or crystals in some embodiments,
U Fentanyl: tests for fentanyl in urine,
U F Gran: refers to granular casts in urine, similar to U C Gran,
U GC RNA: tests for RNA of Neisseria gonorrhoeae (GC), used to diagnose gonorrhea infections in some embodiments,
U Glu: measures glucose levels in urine, which can be a sign of diabetes or other conditions affecting glucose metabolism,
U Hemosiderin: tests for hemosiderin in urine, which can indicate bleeding or iron overload,
U Heroin: tests for heroin metabolites in urine, used in drug screening in some embodiments,
U Hyal Cast: measures hyaline casts in urine, which can be seen in various kidney conditions,
U Hydrocodone: tests for hydrocodone, an opioid medication, in urine,
U Ketones: Measures ketone levels in urine, which can be elevated in conditions like diabetes or starvation,
U K Random: measures potassium levels in random urine samples,
U Leu Est (Leukocyte Esterase): tests for the presence of leukocyte esterase in urine, which can indicate a urinary tract infection,
U Methadone: tests for methadone, a medication used for opioid addiction treatment, in urine,
U Micro: refers to a microbiological test for bacteria or other microorganisms in urine,
U Myoglob: tests for myoglobin in urine, which can be a sign of muscle damage or rhabdomyolysis in some embodiments,
U Na Random: measures sodium levels in random urine samples,
U Nicotine/Cotinine: tests for nicotine or its metabolite cotinine in urine, used to assess smoking or tobacco use,
U Nitrite: tests for nitrite in urine, which can indicate bacterial infection in the urinary tract,
U Opiates: tests for opiates in urine, used in drug screening in some embodiments,
U Osmo Random: measures urine osmolality in random samples, which can provide information about kidney function and hydration in some embodiments,
U Oxycodone: Tests for oxycodone, an opioid medication, in urine,
U pH: measures the pH level of urine, which can indicate various metabolic or renal conditions,
U Protein: tests for the presence of protein in urine, which can be a sign of kidney dysfunction,
U Protein Random: measures protein levels in random urine samples,
U Prot Random: another term for measuring protein in random urine samples,
Ur Alb R (Urine Albumin Random): measures albumin levels in random urine samples, used to assess kidney function,
U RBC (Red Blood Cells): tests for the presence of red blood cells in urine, which can indicate bleeding or injury in some embodiments,
U Renal Tubular Ep: measures renal tubular epithelial cells in urine, which can be a sign of tubular damage or injury,
Uric Acid: measures uric acid levels in urine, which can help diagnose conditions like gout or kidney stones in some embodiments,
Urine Amphetamines (AMP): tests for the presence of amphetamines, used in drug screening to detect stimulant use in some embodiments,
Urine Barbiturates (BAR): detects barbiturates in urine, which are central nervous system depressants,
Urine Benzodiazepines (BZO): tests for benzodiazepines, a class of sedatives and anxiolytics, in urine,
Urine Buprenorphine (BUP): detects buprenorphine, a medication used to treat opioid addiction, in urine,
Urine Cannabinoids (THC): tests for cannabinoids, primarily THC, to detect marijuana use,
Urine Cocaine (COC): detects cocaine metabolites in urine,
Urine Methadone (MTD): tests for methadone, used for pain management and opioid addiction treatment, in urine,
Urine Methamphetamine (mAMP): detects methamphetamine in urine,
Urine Opiates (OPI): tests for opiate metabolites, including morphine and codeine, in urine,
Urine Oxycodone (OXY): detects oxycodone in urine,
Urine Phencyclidine (PCP): Tests for PCP, a hallucinogenic drug, in urine,
Urine Propoxyphene (PPX): detects propoxyphene in urine,
Urine Tricyclic Antidepressants (TCA): tests for tricyclic antidepressants, which are used to treat depression and other conditions, in urine,
Ur Tot Vol (Total Volume): measures the total volume of urine collected, which can provide insights into hydration status and kidney function in some embodiments,
Ur TP UPEP (Total Protein Urine, Urine Protein Electrophoresis): measures total protein and performs electrophoresis to identify different types of proteins in urine, useful for diagnosing conditions like multiple myeloma or nephrotic syndrome in some embodiments,
U Spec Gravity (Specific Gravity): measures the concentration of solutes in urine, indicating kidney function and hydration status,
U Sperm: tests for the presence of sperm in urine, which can be relevant in certain forensic or diagnostic contexts,
U Sq Epi (Squamous Epithelial Cells): measures the number of squamous epithelial cells in urine, which can indicate contamination or certain urinary tract issues,
U TP Elect Random (Urine Total Protein Electrophoresis, Random): similar to Ur TP UPEP but performed on a random urine sample; it separates proteins to help diagnose kidney diseases and other conditions,
U Trans Ep (Transitional Epithelial Cells): measures transitional epithelial cells in urine, which can indicate issues with the urinary tract or bladder,
U Trich (Trichomonas Vaginalis): tests for the presence of Trichomonas vaginalis, a parasite that causes trichomoniasis,
U Trich RNA: detects the RNA of Trichomonas vaginalis for a more sensitive diagnosis of trichomoniasis,
U UrAc Cry (Urine Urate Cry): measures urine urate levels; abnormal levels can indicate conditions like gout or kidney dysfunction,
U Urea Random: measures urea levels in a random urine sample, useful for assessing kidney function and protein metabolism,
U Uric Random: measures uric acid levels in a random urine sample, which helps in diagnosing gout and monitoring kidney function,
U WBC (White Blood Cells): detects white blood cells in urine, which can indicate an infection or inflammation in the urinary tract in some embodiments,
U WB Clump (White Blood Cell Clumps): checks for clumping of white blood cells in urine, which can indicate urinary tract infection or other issues in some embodiments,
U Yeast: tests for the presence of yeast, commonly Candida, in urine which can indicate a fungal infection in some embodiments,
Vanco Random: measures the level of Vancomycin, an antibiotic, in a random blood sample,
Vanco Trough: measures the trough level of Vancomycin, which is the lowest concentration of the drug in the blood before the next dose, involved in dosing and efficacy in some embodiments,
VitaB12 (Vitamin B12): measures vitamin B12 levels, important for red blood cell formation and neurological function,
Vitamin A Level: assesses vitamin A levels, which are involved in vision, immune function, and skin health,
Vit B6 (Vitamin B6): measures vitamin B6 levels, important for metabolism and nerve function,
Vit C (Vitamin C): assesses vitamin C levels, important for collagen synthesis, immune function, and antioxidant protection,
Vit D1,25 DI-OH (Vitamin D, 1,25-dihydroxy): measures the active form of vitamin D, involved in calcium metabolism and bone health,
Vit D2 (Vitamin D2): measures levels of vitamin D2, a form of vitamin D found in supplements and some foods,
VitD 25OH (Vitamin D, 25-hydroxy): measures the storage form of vitamin D, which reflects overall vitamin D status,
Vit D3 (Vitamin D3): measures levels of vitamin D3, which is produced by the skin and found in some foods,
Vit K (Vitamin K): assesses vitamin K levels, important for blood clotting and bone health,
Voriconazole lvl: measures the level of Voriconazole, an antifungal medication, in the blood,
VRP Adenovirus: detects the presence of Adenovirus, which can cause respiratory infections,
VRP Coronavirus: tests for the presence of Coronavirus, which can cause respiratory illness including COVID-19,
VRP Human Metapneumovirus: detects Human Metapneumovirus, which can cause respiratory infections,
VRP Human Rhinovirus/Enterovirus: tests for Human Rhinovirus and Enterovirus, which are common causes of respiratory infections,
VRP Influenza A: detects Influenza A virus, a common cause of seasonal flu,
VRP Influenza A H1: specific test for Influenza A H1 subtype,
VRP Influenza A H1-2009: specific test for Influenza A H1N1 subtype from 2009,
VRP Influenza A H3: specific test for Influenza A H3 subtype,
VRP Influenza B: detects Influenza B virus, another common cause of seasonal flu,
VRP Parainfluenza Virus 1-4: tests for Parainfluenza viruses 1 through 4, which can cause respiratory illnesses in children and adults,
VRP Respiratory Syncytial Virus A and B: detects Respiratory Syncytial Virus types A and B, which are causes of respiratory infections,
VZV IgG Antibody (Varicella-Zoster Virus IgG): tests for antibodies to Varicella-Zoster Virus, indicating past infection or vaccination for chickenpox and shingles,
WBC (White Blood Cells): measures the number of white blood cells in the blood, indicating immune response and infection,
WB INR (Whole Blood INR): measures the International Normalized Ratio (INR) in whole blood, used to monitor blood clotting in patients on anticoagulants,
WB INR Performed at: indicates the location or lab where the whole blood INR test was performed,
WT (Weight): measures body weight, important for dosing medications and assessing overall health,
Zinc Plasma: measures zinc levels in the plasma, involved in various enzymatic functions and immune health, and/or
mean corpuscular hemoglobin concentration (MCHC) (e.g., in units of grams per deciliter).

In some embodiments the plurality of treatment information units 114 comprises scheduling data of the subject, such as how frequently the subject obtains particular diagnostic tests, the dates and times the subject has received medical treatment, surgical intervention schedules, remote visits (remote video/telemedicine consultations, telephonic consultations, etc.) and/or clinical schedules.

In some embodiments, the plurality of treatment information units 114 are acquired in JavaScript Object Notation (JSON) format. In some embodiments the plurality of treatment information units 114 are acquired in Excel file format and/or comma-separated value file format and transformed to JSON format. In some embodiments the plurality of treatment information units 114 of the data repository 106 are stored in JSON) format.

In some embodiments, the plurality of treatment information units 114 are acquired for the data repository 106 through a series of RegEx scans of medical data associated with the subject, such as one or more electronic health records of the subject. As used herein “RegEx scan” refers to the process of using Regular Expressions (RegEx) to search through text data to find patterns, extract specific information, or validate text against a given pattern. For example, the RegEx pattern “\hat{ }[A-Za-z0-9._%+-]+@[A-Za-z0-9.-]+\.[A-Za-z]{2,}$” matches a valid E-mail address.

In some embodiments the plurality of contacts 112 associated with a subject are determined through a series of RegEx scans of the treatment information units 114. In some embodiments, the treatment information units 114 concern treatment data for the subject at particular entity such as through a particular health maintenance organization, at a particular hospital, using a particular medical center, or the like. In some such embodiments there is a master list of health professionals associated with the entity and this list is used to drive a series of RegEx scans, or other forms of scans of the data repository 106 to ascertain which of the health professionals the subject has used, thereby forming the plurality of contacts 112 for the subject. In some embodiments the plurality of contacts comprises 2, 3, 4, 5, 6, 7, 8, 9, 10, 11, 12, 13, 14, 15, 16, 17, 18, 19, 20, or more than 20 contacts associated with treating the subject.

In some embodiments the plurality of conditions 110 associated with a subject are determined through a series of RegEx scans of the treatment information units 114. In some embodiments, the treatment information units 114 are scanned for valid International Classification of Disease, 9th revision, (ICD-9) codes, International Classification of Disease, 10th revision, (ICD-10 codes), and/or current procedure terminology (CPT) code.

CPT codes, maintained by the American Medical Association, describe medical, surgical, and diagnostic services and procedures performed by healthcare providers. For example, the CPT code 99213 is an office or other outpatient visit for the evaluation and management of an established subject.

ICD-9 codes, developed by the World Health Organization (WHO) and used primarily for billing and insurance purposes classify and code diagnoses and medical conditions. For example, the ICD-9 code 401.9 is an unspecified essential hypertension.

ICD-10 codes are the updated version of ICD-9, and provide a more detailed and comprehensive system for classifying diseases and health conditions. For example, the ICD-10 code I10 is Essential (primary) hypertension.

In some embodiments the plurality of first conditions 110 comprises 2, 3, 4, 5, 6, 7, 8, 9, 10, 11, 12, 13, 14, 15, 16, 17, 18, 19, 20, or more than 20 contacts associated with treating the subject.

In some embodiments, the plurality of treatment information units 114 comprises personal data corresponding to the subject and a record of the one or more biological samples obtained (e.g., patient identifiers, patient clinical data, sample type, sample identifiers, cancer conditions, etc.).

In some embodiments, the plurality of treatment information units 114 comprises information related to the specific characteristics of the subject's condition, e.g., somatic variants, epigenetic abnormalities, associated oncogenic pathogenic infections, and/or pathology abnormalities.

In some embodiments, the plurality of treatment information units 114 comprises disease states including, but not limited, to depression, diabetes, Parkinson's, Alzheimer's, cancer, etc.

In some embodiments the plurality of treatment information units 114 comprises information obtained from a liquid biopsy of a subject. For example, in some embodiments the information obtained from a liquid biopsy of a subject comprises a corresponding nucleic acid sequence of each cell-free DNA fragment in a plurality of cell-free DNA fragments that is obtained from a plurality of sequence reads of a sequencing reaction of a plurality of cell-free DNA fragments from the liquid biopsy sample of the test subject. In some such embodiments, each respective nucleic acid sequence in the plurality of nucleic acid sequences is accompanied by a methylation pattern for a corresponding cell-free DNA fragment in the plurality of cell-free DNA fragments.

In some embodiments, the liquid biopsy sample is blood. In some embodiments, the liquid biopsy sample comprises blood, whole blood, peripheral blood, plasma, serum, or lymph of the subject.

In some embodiments, the plurality of treatment information units 114 comprises information about one or more of the biological samples obtained from the patient. In some embodiments at least one of these biological samples is a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, one or more blood samples are collected from a subject in commercial blood collection containers. In some embodiments, saliva samples are collected from patients in commercial saliva collection containers.

In some embodiments, the volume of the liquid biopsy sample is less than 30 mL. In some embodiments, the volume of the liquid biopsy sample is from 1 mL to 50 mL, from 2 mL to 40 mL, from 3 mL to 35 mL, or from 5 mL to 31 mL. For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater.

Liquid biopsy samples include cell free nucleic acids, including cell-free DNA (cfDNA). cfDNA isolated from subjects that have cancer includes DNA originating from cancerous cells, also referred to as circulating tumor DNA (ctDNA), cfDNA originating from germline (e.g., healthy or non-cancerous) cells, and cfDNA originating from hematopoictic cells (e.g., white blood cells). The relative proportions of cancerous and non-cancerous cfDNA present in a liquid biopsy sample varies depending on the characteristics (e.g., the type, stage, lineage, genomic profile, etc.) of the subject's cancer.

In some embodiments, cell-free DNA is isolated from the biological sample from the subject using commercially available reagents, including digestion with proteinase K. In some embodiments, the selective binding properties of a silica membrane are used to extract cell-free DNA from the biological sample from the subject using circulating nucleic acid kits. In some such embodiments, the biological sample is lysed in an optimized buffer and adjusted to binding conditions. Then, the biological sample is loaded directly onto a spin column. In this step, cell-free DNA is bound to the silica membrane, and contaminants are removed in wash steps. Finally, pure cell-free DNA is eluted in small volumes of a low-salt buffer for downstream applications. See, Hai et al., 2022, “Whole-genome circulating tumor DNA methylation landscape reveals sensitive biomarkers of breast cancer,” MedComm (2020) September 3(3): e134, which is hereby incorporated by reference.

In some embodiments, adapters such as unique dual index (UDI) adapters are ligated onto the cell-free DNA fragments. In some embodiments, adapters with unique molecular indices (UMI), which are short nucleic acid sequences (e.g., 4-10 base pairs), are ligated onto the cell-free DNA fragments. In some embodiments, the UDI adapters include UMIs. In some embodiments, UMIs are degenerate base pairs that serve as a unique tag that can be used to identify sequence reads originating from a specific DNA fragment. In some embodiments, e.g., when multiplex sequencing will be used to sequence cell-free DNA fragments from a plurality of samples (e.g., from the same or different subjects) in a single sequencing reaction, a subject-specific index is also added to the nucleic acid molecules. In some embodiments, the subject-specific index is a short nucleic acid sequence (e.g., 3-20 nucleotides) that are added to ends of cell-free DNA fragments during library construction, that serve as a unique tag that can be used to identify sequence reads originating from a specific subject sample. Examples of identifier sequences are described, for example, in Kivioja et al., Nat. Methods 9 (1): 72-74 (2011) and Islam et al., Nat. Methods 11 (2): 163-66 (2014), the contents of which are hereby incorporated by reference, in their entireties, for all purposes.

In some embodiments, a biopsy from a subject is used to perform a sequencing reaction that is a whole genome methylation sequencing or targeted panel sequencing. Regardless of whether a targeted, probe based methylation sequencing or a whole genome methylation sequencing is performed, in some embodiments nucleic acids isolated from the biological sample (e.g., cfDNA) are treated to convert unmethylated cytosines to uracils. Accordingly, in such embodiments, when the nucleic acids are sequenced, all cytosines called in the sequencing reaction were necessarily methylated, since the unmethylated cytosines were converted to uracils and accordingly would have been called as thymidines, rather than cytosines, in the sequencing reaction. Commercial kits are available for bisulfite-mediated conversion of methylated cytosines to uracils. Commercial kits are also available for enzymatic conversion of methylated cytosines to uracils.

In some embodiments, the cell-free DNA fragments are amplified and purified using commercial reagents.

In some such embodiments, the concentration and/or quantity of the cell-free DNA fragments are quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified cell-free DNA fragments is sequenced.

In some embodiments sequencing is performed on a next generation sequencer (e.g., an Illumina HiSeq 4000, Illumina NovaSeq 6000, Oxford Nanopore, Biomodal) to a unique on-target depth selected by the user. In some embodiments, sequencing is performed using sequencing-by-synthesis technology (Illumina), pyrosequencing (454 Life Sciences), ion semiconductor technology (Ion Torrent sequencing), single-molecule real-time sequencing (Pacific Biosciences), or sequencing by ligation (SOLID sequencing).

In some embodiments, a plurality of sequence reads is obtained from such sequencing and comprises at least 50,000 sequence reads or at least 250,000 sequence reads. In some embodiments the plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the plurality of sequence reads consists of between 50,000 sequence reads and 10 million sequence reads. In some embodiments, the plurality of sequence reads consists of between 100,000 sequence reads and 8 million sequence reads. In some embodiments, the plurality of sequence reads consists of between 200,000 sequence reads and 6 million sequence reads.

In some embodiments a panel-based sequencing reaction of a plurality of loci is performed using a biopsy obtained from the subject. In some such embodiments, a plurality of nucleic acid probes (e.g., a probe set) is used to enrich one or more target sequences in the biopsy sample for the plurality of loci. In some embodiments, the probe set includes probes targeting one or more gene loci, e.g., exon or intron loci within the plurality of loci. In some embodiments, the plurality of loci enriched by the probe set includes one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci.

In some embodiments, the plurality of loci includes one more of the genes listed in Table 1. In some embodiments, the plurality of loci includes at least 5 of the genes listed in Table 1. In some embodiments, the plurality of loci includes at least 10 of the genes listed in Table 1. In some embodiments, the plurality of loci includes at least 25 of the genes listed in Table 1. In some embodiments, the plurality of loci includes at least 50 of the genes listed in Table 1. In some embodiments, the plurality of loci includes at least 75 of the genes listed in Table 1. In some embodiments, the plurality of loci includes at least 100 of the genes listed in Table 1. In some embodiments the plurality of loci consists of or comprises all of the genes listed in Table 1.

Table 1. An example panel of 105 genes.

TABLE 1
Liquid Biopsy Gene Panel

| | | | | | | |
|---|---|---|---|---|---|---|
| ALK | B2M | ERRFI1 | IDH2 | MSH6 | PIK3R1 | SPOP |
| FGFR2 | BAP1 | ESR1 | JAK1 | MTOR | PMS2 | STK11 |
| FGFR3 | BRCA1 | EZH2 | JAK2 | MYCN | PTCH1 | TERT |
| NTRK1 | BRCA2 | FBXW7 | JAK3 | NF1 | PTEN | TP53 |
| RET | BTK | FGFR1 | KDR | NF2 | PTPN11 | TSC1 |
| ROS1 | CCND1 | FGFR4 | KEAP1 | NFE2L2 | RAD51C | TSC2 |
| BRAF | CCND2 | FLT3 | KIT | NOTCH1 | RAF1 | UGT1A1 |
| AKT1 | CCND3 | FOXL2 | KRAS | NPM1 | RB1 | VHL |
| AKT2 | CDH1 | GATA3 | MAP2K1 | NRAS | RHEB | CCNE1 |
| APC | CDK4 | GNA11 | MAP2K2 | PALB2 | RHOA | CD274 |
| AR | CDK6 | GNAQ | MAPK1 | PBRM1 | RIT1 | EGFR |
| ARAF | CDKN2A | GNAS | MLH1 | PDCD1LG2 | RNF43 | ERBB2 |
| ARID1A | CTNNB1 | HNF1A | MPL | PDGFRA | SDHA | MET |
| ATM | DDR2 | HRAS | MSH2 | PDGFRB | SMAD4 | MYC |
| ATR | DPYD | IDH1 | MSH3 | PIK3CA | SMO | KMT2A |

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cell-free DNA fragment can contain a sequence that is complementary to either strand, because the cell-free DNA fragments are double stranded. In some embodiments, each probe in the plurality of probes includes a nucleic acid sequence that is identical or complementary to at least 10, at least 11, at least 12, at least 13, at least 14, or at least 15 consecutive bases of a locus in the plurality of loci. In some embodiments, each probe in the plurality of probes includes a nucleic acid sequence that is identical or complementary to at least 20, 25, 30, 40, 50, 75, 100, 150, 200, or more consecutive bases of a locus in the plurality of loci.

In some embodiments, the plurality of loci is a whole-exome panel comprising the exomes of the biopsy. In some embodiments, the plurality of loci is a whole-genome panel that comprises the genome of a specimen of the subject.

In some embodiments, the probes for the panel-based sequencing region include additional nucleic acid sequences that do not share any homology to the plurality of loci. For example, in some embodiments, the probes also include nucleic acid sequences containing an identifier sequence, e.g., a unique molecular identifier (UMI), e.g., that is unique to a particular sample or to the subject. Examples of identifier sequences are described, for example, in Kivioja et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to a nucleic acid molecule that is complementary to a locus in the plurality of loci, for recovering a cell-free DNA fragment. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

In some embodiments, panel-targeting sequencing is performed to an average on-target depth of at least 500×, at least 750×, at least 1000×, at least 2500×, at least 500×, at least 10,000×, or greater depth.

In some embodiments, the plurality of loci is sequenced at an average sequence depth of at least 250× by the second sequencing reaction. In some embodiments, the plurality of loci is sequenced at an average sequence depth of at least 1000× by the second sequencing reaction. In some embodiments, the plurality of loci is sequenced at an average sequence depth of at least 500×, at least 750×, at least 1000×, at least 2500×, at least 500×, at least 10,000×, or greater.

In some embodiments, the plurality of sequence reads obtained by the panel-based sequencing comprises at least 50,000 sequence reads or at least 250,000 sequence reads. In some embodiments the plurality of sequence reads obtained by the panel-based sequencing comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the second plurality of sequence reads consists of between 50,000 sequence reads and 10 million sequence reads. In some embodiments, the plurality of sequence reads obtained by the panel-based sequencing consists of between 100,000 sequence reads and 8 million sequence reads. In some embodiments, the plurality of sequence reads obtained by the panel-based sequencing consists of between 200,000 sequence reads and 6 million sequence reads.

In some embodiments each respective sequence read in the plurality of sequence reads obtained by the panel-based sequencing is mapped to one or more reference sequences (e.g., a reference human genome) by identifying a sequence in a region of the one or more reference sequences that best matches the sequence of nucleotides in the respective sequence read. In some embodiments, this mapping uses methods known in the art to determine alignment position information. The alignment position information may indicate a beginning position and an end position of a region in the one or more reference sequences that corresponds to a beginning nucleotide base and end nucleotide base of the respective sequence read. Alignment position information may also include the sequence read length that can be determined from the beginning position and end position. A region in the one or more reference sequences may be associated with a gene or a segment of a gene. Any of a variety of alignment tools can be used to optimize the mapped alignment.

Block 204. Referring to block 204, in some embodiments, the plurality of processor executable instructions further comprises instructions for updating the data repository using multimodal treatment data for the subject.

Multimodal treatment data refer to patient health records that integrate data from multiple types of treatments and diagnostic modalities. These records are designed to provide a comprehensive view of a subject's health by incorporating various sources of information, including clinical notes, diagnostic images, laboratory results, and treatment plans.

In some embodiments, the multimodal treatment data comprises clinical data including physician notes, patient history, and/or physical examination findings.

In some embodiments, the multimodal treatment data comprises diagnostic images such as X-rays, magnetic resonance imaging, computed tomography scans, and/or ultrasounds.

In some embodiments, the multimodal treatment data comprises laboratory test results such as blood tests, urine tests, biopsy results, and/or other laboratory data.

In some embodiments, the multimodal treatment data comprises treatment plans such as information on prescribed medications, surgical procedures, physical therapy, or other treatments.

For example, a subject with a chronic kidney disease (CKD) might have a multimodal treatment health record that includes: clinical notes from nephrologists and primary care physicians, blood test results showing kidney function markers like creatinine and glomerular filtration rate (GFR), imaging studies such as renal ultrasounds or CT scans to assess kidney structure, medication records detailing prescriptions for managing CKD and its complications, and records of dialysis sessions or other treatments if applicable.

Block 206. Referring to block 206, in some embodiments, the one or more first conditions 110 comprises a first diagnosis associated with the subject.

In some such embodiments the first diagnosis is for cancer condition. A cancer condition refers to a characteristic of a subject's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject further describe the cancer state or cancer condition of the subject, e.g., age, gender, weight, race, personal habits (e.g., smoking, drinking, diet), other pertinent medical conditions (e.g., high blood pressure, dry skin, other diseases, etc.), current medications, allergies, pertinent medical history, current side effects of cancer treatments and other medications, etc.

In some embodiments, the cancer condition is a particular type of cancer. In some embodiments, the cancer condition is lung cancer (e.g., non-small-cell lung cancer). In some embodiments, the cancer condition is breast cancer. Additional non-limiting examples of cancer conditions include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, non-clear cell renal cell carcinoma, glioblastoma, glioma, kidney cancer, gastrointestinal stromal tumor, medulloblastoma, bladder cancer, gastric cancer, bone cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, HER2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

In some embodiments, the cancer condition is a particular stage of a particular type of cancer. In some such embodiments, the stage of the particular type of cancer is a stage of cancer the subject was diagnosed with prior to treatment. Cancer is typically staged to determine the extent of its spread and to guide treatment decisions. The stage of cancer refers to the extent to which it has grown and spread from its original location. Each cancer has its own criteria for determine stage but generally relies on a determination of the size of the primary tumor (T) and whether it has invaded nearby tissues, evaluation of lymph node involvement (N) to find indications of whether cancer has spread to nearby lymph nodes, and assessment of distant metastasis (M), which indicates whether the cancer has spread to distant organs or tissues. Metastasis means that cancer has spread from the primary site to other parts of the body. In some embodiments the staging system used is the cancer TNM system, which combines the T, N, and M information to assign a stage. In some embodiments the stages are denoted using Roman numerals (I, II, III, IV) and may have subcategories (e.g., stage IIA, stage IIB) to provide more precise information. In a brief overview of these stages, in stage 0, the cancer is in situ, meaning it is confined to the layer of cells where it began and has not invaded nearby tissues, in stage I: the cancer is localized and small in size, in stage II, the cancer may be larger and/or have spread to nearby lymph nodes, but it is still relatively localized, in stage III, the cancer has typically spread further into nearby tissues and may involve more lymph nodes, in stage IV, the cancer has spread to distant organs or tissues, indicating metastasis. This is often the most advanced stage. The specific criteria for each stage can vary depending on the type of cancer. See, for example, details of TNM staging for breast cancer in Part et al., 2011, “Clinical relevance of TNM staging system according to breast cancer subtypes,” Annals of Oncology 22(7), pp. 1554-1560, which is hereby incorporated by reference. Additionally, some cancers have their own staging systems tailored to their characteristics. See the Internet at www.cancer.gov/about-cancer/diagnosis-staging/staging.

Referring to block 208, in some embodiments, the plurality of contacts 112 associated with the subject comprises a third party sponsoring a clinical trial. Suitable clinical trials in accordance with some embodiments of the present disclosure are found at the United States National Institute of Health clinical center webpage. An example of a sponsor of a clinical trial is the National Institute of Diabetes and Digestive and Kidney Diseases (NIDDK). The NIDDK is a part of the National Institutes of Health (NIH) in the United States. The NIDDK sponsors and conducts medical research and clinical trials to improve understanding, prevention, and treatment of diseases related to diabetes, digestive disorders, and kidney diseases.

Referring to block 210, in some embodiments, the plurality of contacts 112 associated with the subject includes the subject. For instance, in some embodiments the plurality of contacts includes contact information, such as an E-mail address or phone number, of the subject.

Referring to block 212, in some embodiments, the plurality of contacts 112 associated with the subject includes a plurality of caregivers that treat the subject at a first institution. For example, in some embodiments, the treatment information units 114 concern treatment data for the subject at particular institution.

In some embodiments, the institution is a hospital (e.g., a general hospital, a specialty hospital, a teaching hospital, etc.), a clinic (e.g., a primary care clinic, a specialty clinic, a community health center, etc.), a long-term care facility (e.g., a nursing home, an assisted living facility, a rehabilitation center, etc.), an outpatient care center (e.g, an ambulatory surgery center, an urgent care center, a diagnostic imaging center, etc.), a home health care agency (e.g., provides medical and personal care services to patients in their homes, including nursing care, physical therapy, and assistance with daily activities, etc.), a mental health care facility (e.g., a psychiatric hospital, an outpatient mental health clinic, etc.), a hospice or palliative care facility (e.g., that provides end-of-life care focused on comfort and quality of life for subjects with terminal illnesses, etc.), a public health institution (e.g., a government health department, a community health organization, etc.), a private practice (e.g., individual or group practices run by physicians, dentists, or other healthcare professionals, providing specialized or primary care services), a research institution (e.g., that conducts medical research to advance knowledge in various fields of healthcare and develop new treatments and technologies, etc.), a pharmacy (e.g., a community pharmacy, a hospital pharmacy, etc.), an alternative and complementary medicine center (e.g., offer treatments such as acupuncture, chiropractic care, naturopathy, and herbal medicine, etc.), a particular health maintenance organization, and/or a particular preferred provider organization, etc., or any combination thereof.

In some such embodiments there is a master list of health professionals associated with the institution and this list is used to drive a series of scans of the data repository 106 to ascertain which of the health professionals the subject has used, thereby forming the plurality of caregivers that treat the subject. In some embodiments the plurality of caregivers that treat the subject comprises 2, 3, 4, 5, 6, 7, 8, 9, 10, 11, 12, 13, 14, 15, 16, 17, 18, 19, 20, or more than 20 caregivers associated with treating the subject. Non-limiting examples of such caregivers include, but are not limited to, doctors (e.g., general practitioners and/or specialists such as cardiologists, dermatologists, endocrinologists, neurologists, oncologists, pediatricians, psychiatrists, surgeons, etc.), nurses (e.g., registered nurses, nurse practitioners, limited practical nurses, etc.), physician assistants, dentists, (e.g. general dentists, specialists such as orthodontists, periodontist, endodontists, oral surgeons, etc.), pharmacists, physical therapists, occupational therapists, speech-language pathologists, radiologic technologists, medical laboratory technologists, mental health professionals (e.g., psychologist, psychiatrists, etc.), chiropractors, acupuncturests, naturopathic doctors (e.g., focus on holistic and natural treatments, including herbal medicine and lifestyle counseling), optometrists, ophthalmologists, and podiatrists that have participated in the treatment of the one or more of the conditions of the subject.

Block 214. Referring to block 214, the plurality of processor executable instructions further comprises instructions for discovering a first subset of decision rules from among a plurality of decision rules 118 through a modeling procedure that aligns a first condition 110 in the one or more first conditions against the plurality of decision rules. Each decision rule 118 in the first subset of decision rules has a corresponding triggering condition 120. The first subset of decision rules models a response to the first condition at a first entity, such as one of the institutions described above.

In some embodiments, there are at least 5 decision rules, at least 2500 decision rules, at least 5000 decision rules, at least 7000 decision rules, at least 8000 decision rules, at least 10000 decision rules, at least 12000 decision rules, at least 14000 decision rules, at least 15000 decision rules, at least 18000 decision rules, at least 20000 decision rules, at least 30000 decision rules, at least 40000 decision rules, at least 50000 decision rules, at least 60000 decision rules, at least 70000 decision rules, at least 80000 decision rules, at least 100000 decision rules, at least 1×10⁶ decision rules or at least 1×10⁷ decision rules.

Discovering a subset of decision rules from among the plurality of decision rules makes use of a condition in the 110 in the plurality of conditions associated with the subject. For example, when the condition is diabetes, only those decision rules that could impact or relate to diabetes are considered candidates for the subset of decision rules that is ultimately selected in accordance with block 214.

A condition 114 can be described as a multidimensional tensor, where the dimensions represent different aspects of the condition. For example, the condition “diabetes” can include dimensions of severity, subject age, complicating factors, etc. For each of these dimensions, a value representative of the subject for that dimension is included in the tensor. Identification of the subset of decision rules, then, from among the plurality of decision rules requires using the condition (e.g, the tensor describing the condition) as a search query against the plurality of decision rules that best matches the condition. Each of the decision rules in the subset of decision rules that is eventually discovered in accordance with block 214 is associated with one or more of the dimensions of the query condition.

On the scale of the dimensions and number of decision rules available in typical embodiments, such a search cannot be mentally performed.

Consider as a first example, a condition, described by a query tensor, where the query tensor has values for 10 dimensions out of a possible 100 dimensions. For example, the condition is diabetes, and the ten dimensions include dimensions such as (i) fasting blood sugar level in the past month, (ii) oral glucose tolerance, (iii) hemoglobin A1c level, (iv) random plasma glucose test results, (v) body mass index, (vi) waist circumference, (vii) blood pressure, (viii) lipid profile, (ix) age, and (x) smoking status. In some embodiments, the subject will have values for each of these dimensions. The goal is to search for a combination of decision rules, drawn from among 1000 decision rules, that collectively best match the query tensor. In this example each decision rule has no more than 5 dimensions in the 100 dimensions, and so only combinations of 2 or more decision rules are considered during the search. For example, a particular decision rule may be associated with a single dimension (e.g., take a hemoglobin A1c level test). Another decision rule may be associated with a two dimensions (e.g., obtain body mass index and weight of subject). If a brute force exhaustive algorithm is used to find the best combination of decision rules that collectively best match a query tensor with values for 10 out of 100 dimensions, where each decision rule involves no more than 5 dimensions, and combinations of 2 or more decision rules are considered, the number of combinations of decision rules to be considered is:

$${\sum }_{k = 2}^{1000} ( \frac{1000}{k} ) = {2}^{1000} - ( \frac{1000}{0} ) - ( \frac{1000}{1} ) \approx {2}^{1000}$$

This is a very large number, approximately 2¹⁰⁰⁰, which is more than 10³⁰⁰. Each combination needs to be evaluated against the query tensor, which involves comparing the selected dimensions of the query tensor with those covered by each decision rule in the combination. In some embodiments, rather than a brute force methods, algorithms such as dynamic programming, heuristic search, or genetic algorithms are employed to manage this combinatorial problem thereby reducing the search space and focusing on promising solutions early in the search process. Moreover, in some embodiments various subsets of decision rules are already clustered together to form treatment plans by an institution as discussed in further detail below. In some such embodiments only such clusters need be evaluated against a condition of the subject.

Consider as a second example, a condition, described by a query tensor, where the query tensor has values for 6 dimensions out of a possible 50 dimensions. For example, the condition is Alzheimer's disease, and the 6 dimensions include dimensions such as (i) a mini-mental state examination score, (ii) a Montreal cognitive assessment score, (iii) an Alzheimer's disease assessment scale-cognitive subscale (ADAS-Cog) score, (iv) an activities of daily living (ADL) assessment, (v) an instrumental activities of daily living (IADL) test result, and (vi) a clinical dementia rating. In some embodiments, the subject will have values for each of these dimensions. The goal is to search for a combination of decision rules, drawn from among 500 decision rules, that collectively best match the query tensor. In this example, each decision rule has no more than 3 dimensions in the 50 dimensions, and so only combinations of 2 or more decision rules are considered during the search. For example, a particular decision rule may be associated with a single dimension (e.g., perform an annual inspection that determines an ADAS-Cog score). Another decision rule may be associated with a two dimensions (e.g., perform an annual inspection that determines a clinical dementia rating and performs magnetic resonance imaging). If a brute force exhaustive algorithm is used to find the best combination of decision rules that collectively best match a query tensor with values for 6 out of 50 dimensions, where each decision rule involves no more than 3 dimensions, and combinations of 2 or more decision rules are considered, the number of combinations of decision rules to be considered can be calculated using the binomial coefficient

$$( \frac{500}{k} ) .$$

The total number or combinations of 2 or more decision rules is given by:

$${\sum }_{k = 2}^{500} ( \frac{500}{k} ) = {2}^{500} - ( \frac{500}{0} ) - ( \frac{500}{1} ) = {2}^{500} - 1 - 500 \approx {2}^{500}$$

This is a very large number, approximately 2500, which is approximately 3.27×10¹⁵⁰. Each combination needs to be evaluated against the query tensor, which involves comparing the selected dimensions of the query tensor with those covered by each decision rule in the combination. In some embodiments, rather than a brute force methods, algorithms such as dynamic programming, heuristic search, or genetic algorithms are employed to manage this combinatorial problem thereby reducing the search space and focusing on promising solutions early in the search process. Moreover, in some embodiments various subsets of decision rules are already clustered together to form treatment plans by an institution as discussed in further detail below. In some such embodiments only such clusters need be evaluated against a condition of the subject.

In some embodiments a condition has values, or is otherwise associated with 2, 3, 4, 5, 6, 7, 8, 9, 10, 11, 12, 13, 14, 15, 16, 17, 18, 19, 20, 21, 22, 23, 24 or 25 dimensions.

In some embodiments each such condition is out of a possible 100 or more dimensions, 200 or more dimensions, 300 or more dimensions, 400 or more dimensions, 500 or more dimensions, 600 or more dimensions, 700 or more dimensions, 800 or more dimensions, 900 or more dimensions, 1000 or more dimensions, 2000 or more dimensions, 3000 or more dimensions, 4000 or more dimensions, 5000 or more dimensions, 10,000 or more dimension, 100,000 or more dimensions, or 1×10⁶ or more dimensions.

In some embodiments, the decision rule data store 116 comprises 100 or more decision rules, 200 or more decision rules, 300 or more decision rules, 400 or more decision rules, 500 or more decision rules, 600 or more decision rules, 700 or more decision rules, 800 or more decision rules, 900 or more decision rules, 1000 or more decision rules, 2000 or more decision rules, 3000 or more decision rules, 4000 or more decision rules, 5000 or more decision rules, 10,000 or more decision rules, 100,000 or more decision rules, or 1×10⁶ or more decision rules.

In some embodiments, each decision rule 118 is associated with no more than 1 dimension represented by the data repository 106. In some embodiments, each decision rule 118 is associated with no more than 2 dimensions represented by the data repository 106. In some embodiments, each decision rule 118 is associated with no more than 3 dimensions represented by the data repository 106. In some embodiments, each decision rule 118 is associated with no more than 4 dimensions represented by the data repository 106. In some embodiments, each decision rule 118 is associated with no more than 5 dimensions represented by the data repository 106. In some embodiments, each decision rule 118 is associated with no more than 6, 7, 8, 9, 10, 11, 12, 13, 14, 15, 16, 17, 18, 19, or 20 dimensions represented by the data repository 106.

Block 216. Referring to block 216, in some embodiments, the plurality of decision rules includes a plurality of subsets of decision rules. Each respective subset of decision rules in the plurality of subsets of decision rules models a respective institution based care plan for a diagnosis in a plurality of diagnoses. In some embodiments, the diagnosis is one of the conditions 110 of the subject identified in the data repository 106.

For instance, consider the case where the diagnosis is that the subject has stage 2 pancreatic cancer in which the cancer originated in the head of the pancreas but the subject is otherwise healthy. In this instance, a subset of decision rules associated with treatment of the subject may include a decision rule that specifies a Whipple Procedure (Pancreaticoduodenectomy). A Whipple Procedure is a primary surgical option for tumors located in the head of the pancreas. It involves removing the head of the pancreas, part of the small intestine (duodenum), the gallbladder, part of the bile duct, and sometimes a portion of the stomach. Nearby lymph nodes are also removed to check for cancer spread. The subset of decision rules associated with treatment of the subject may further include another decision rule that specifies performing post-surgery chemotherapy to reduce the risk of cancer reoccurrence. In some instances, such post-surgery chemotherapy is performed using Gemcitabine, 5-Fluorouracil (5-FU) combined with leucovorin, or FOLFIRONX (a combination of 5-FU, leucovorin, irinotecan, and oxaliplatin). In some embodiments, the subset of decision rules includes another decision rule that specifies pre-surgery chemotherapy or chemoradiation. In some instances, such chemotherapy or chemoradiation is given before surgery to shrink the tumor and make it easier to remove. This is more common if the tumor is borderline resectable or if there is a concern about involvement with surrounding blood vessels. In some embodiments, the subset of decision rules includes another decision rule that specifies chemoradition therapy. In some instances, such radiation therapy may be used in combination with chemotherapy either before surgery (neoadjuvant) to shrink the tumor or after surgery (adjuvant) to kill any remaining cancer cells. In some embodiments the subset of decision rules includes another decision rule that specifies some form of follow-up monitoring. For instance, there may be decision rules specifying taking a physical examination, a blood test (e.g., including for the CA 19-9 tumor marker), a follow up imaging study, and/or an endoscopic procedure. In some embodiments the subset of decision rules includes another decision rule that specifies supportive care, such as nutritional support, pain management, and/or psychological support. In some embodiments the subset of decision rules includes another decision rule that specifies a clinical trial that the subject is eligible to participate in based on the diagnosis.

In some embodiments, the plurality of decision rules comprises 10 or more subsets of decision rules where each such subset of decision rules is associated with a condition 110, such as a diagnosis, in the plurality of conditions. In some embodiments the plurality of decision rules comprises 20 or more, 30 or more, 40 or more, 50 or more, 60 or more, 70 or more, 80 or more, 90 or more, or 100 or more subsets of decision rules where each such subset of decision rules is associated with a condition 110, such as a diagnosis, in the plurality of conditions. In some embodiments the plurality of decision rules comprises 200 or more, 300 or more, 400 or more, 500 or more, 600 or more, 700 or more, 800 or more, 900 or more, or 1000 or more subsets of decision rules where each such subset of decision rules is associated with a condition 110, such as a diagnosis, in the plurality of conditions.

In some embodiments, the plurality of decision rules comprises 10 or more subsets of decision rules, where each such subset of decision rules is associated with 2, 3, 4, or 5 or more conditions 110, such as diagnoses, in the plurality of conditions. In some embodiments, the plurality of decision rules comprises 20 or more, 30 or more, 40 or more, 50 or more, 60 or more, 70 or more, 80 or more, 90 or more, or 100 or more subsets of decision rules, where each such subset of decision rules is associated with 2, 3, 4, or 5 or more conditions 110, such as diagnoses, in the plurality of conditions. In some embodiments the plurality of decision rules comprises 200 or more, 300 or more, 400 or more, 500 or more, 600 or more, 700 or more, 800 or more, 900 or more, or 1000 or more subsets of decision rules, where each such subset of decision rules is associated with 2, 3, 4, or 5 or more conditions 110, such as diagnoses, in the plurality of conditions.

In some embodiments each subset of decision rules represents clinical guidelines for a disease or condition published by an institution such as the American Medical Association (AMA), the World Health Organization (WHO), the Centers for Disease Control and Prevention (CDC), the National Institute for Health and Care Excellence (NICE), the American College of Physicians (ACP), the American Heart Association (AHA), the American College of Cardiology (ACC), the European Society of Cardiology (ESC), the Heart Rhythm Society (HRS), the American College of Clinical Pharmacy (ACCP), the American College of Surgeons (ACS), the Society of Interventional Radiology (SIR), the American Academy of Pediatrics (AAP), the American Society of Anesthesiologists (ASA), etc. In some embodiments, each subset of decision rules represents clinical guidelines for a disease or condition published by an institution such as the American Medical Association (AMA), the World Health Organization (WHO), the Centers for Disease Control and Prevention (CDC), the National Institute for Health and Care Excellence (NICE), the American College of Physicians (ACP), the American Heart Association (AHA), the American College of Cardiology (ACC), the European Society of Cardiology (ESC), the Heart Rythym Society (HRS), the American College of Clinical Pharmacy (ACCP), the American College of Surgeons (ACS), the Society of Interventional Radiology (SIR), the American Academy of Pediatrics (AAP), the American Society of Anesthesiologists (ASA), etc, as augmented or modified by the first entity (e.g., to include relevant medical staff at the first entity, etc.).

Block 218. Referring to block 218, in some embodiments, the plurality of decision rules model respective institution based care plans for more than 50 diagnoses. The example of a pancreatic cancer diagnosis was provided above in conjunction with block 216.

In some embodiments, a subset of the plurality of decision rules models a respective institution based care plan for a diagnosis for a cancer, a hematologic disorder, an autoimmune disease, an inflammatory disease, an immunological disorder, a metabolic disorder, a neurological disorder, a genetic disorder, a psychiatric disorder, a gastroenterological disorder, a renal disorder, a cardiovascular disorder, a dermatological disorder, a respiratory disorder, a viral infection, or another disease or disorder.

In some embodiments, a subset of the plurality of decision rules models a respective institution based care plan for diagnosis that is a cardiovascular disorder, a respiratory disorder, a neurological disorder, a gastrointestinal disorder, a hepatobiliary disorder, a musculoskeletal disorder, a renal or urologic disorder, an immune disorder, a hematological disorder, a metabolic or nutritional disorder, an endocrine disorder, an eye disorder, an car, nose and throat disorder, a skin disorder, a malignant neoplasm, an infection, trauma, a genetic disorder, an obstetric or gynecologic disorder, a sexual disorder, or a psychiatric disorder.

In some embodiments the plurality of decision rules models respective institution based care plans for a diagnosis that is a disease or condition described in Williams and Wilkins, Professional Guide to Diseases, 9^{th} Edition, Wolters Kluwer/Lippincott Williams & Wilkins, 2009, which is hereby incorporated by reference.

In some embodiments, the plurality of decision rules model respective institution based care plans for more than 75 diagnoses, 100 diagnoses, 150 diagnoses, 200 diagnoses, 250 diagnoses, 300 diagnoses, 500 diagnoses, or 1000 diagnoses.

In some embodiments each diagnosis factors in, as part of a treatment plan for the diagnosis, of any number of covariates, such as subject age, weight, body mass index, smoking status, sex, race, allergies, and biological markers.

Block 220. Referring to block 220, in some embodiments, the plurality of decisions rules is in the form of a directed graph comprising a plurality of nodes and a plurality of edges. Each respective node in the plurality of nodes is a decision rule in the plurality of decision rules. Each edge in the plurality of edges links a respective first node in the plurality of nodes with at least one other respective second node in the plurality of nodes and specifies what conditions are to be satisfied by the data in the data repository in order to include the decision rule of the at least one other respective second node in the first subset of decision rules when the decision rule of the first respective node is included in the first subset of decision rules. The instructions for discovering the first subset of decision rules from among the plurality of decision rules through a modeling procedure that aligns a first condition in the one or more first conditions against the plurality of decision rules comprises traversing the directed graph beginning at an initial node in the directed graph.

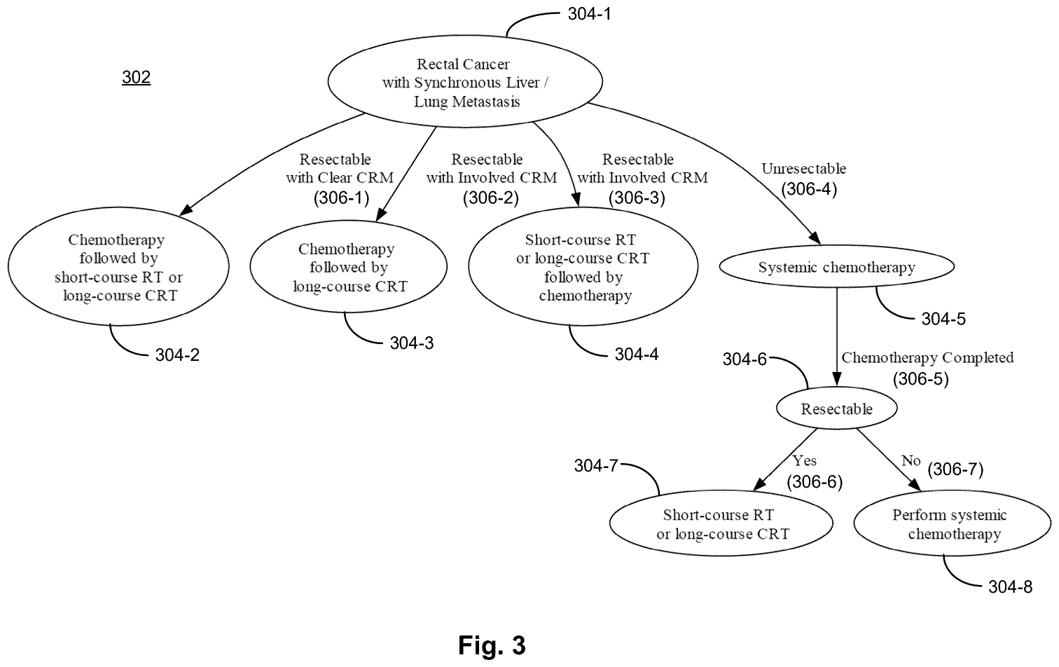

FIG. 3 illustrates. FIG. 3 is a directed graph 302 of a treatment plan for stage IV rectal cancer with synchronous liver/lung metastasis. The treatment plan illustrated in FIG. 3 is modeled from a standard of care for this diagnosis described in Dominguez et al., “Stage IV Colorectal Cancer Management and Treatment,” Journal of Clinical Medicine 2023, 12, 2072, which is hereby incorporated by reference. In FIG. 3, the treatment plan has been encoded as a series of decision rules in the form of a directed graph 302.

Directed graph 302 includes a plurality of nodes 304 and a plurality of edges 306. Each respective node 304 is a decision rule 118 in the plurality of decision rules. Each edge 306 in the plurality of edges links a respective first node in the plurality of nodes with at least one other respective second node in the plurality of nodes and specifies what conditions are to be satisfied by the data in the data repository 106 in order to include the decision rule of the at least one other respective second node in the first subset of decision rules when the decision rule of the first respective node is included in the first subset of decision rules. For example, decision rule 304-1 specifies the condition of rectal cancer with synchronous lung liver or lung metastasis. Edge 306-1 specifies that the data in the data repository 106 is to include an indication that the tumor is resectable and with a clear circumferential resection margin (CRM). If this is the case (e.g., the data repository 106 includes documentation that the tumor is in fact resectable with a clear CRM), decision rule 304-2 (chemotherapy followed by short-course radiotherapy) is included in the subset of decision rules. Edges 306-2 and 306-3 each specify that the data in the data repository 106 is to include an indication that the tumor is resectable but with an involved circumferential resection margin (CRM). If this is the case (e.g., the data repository 106 includes documentation that the tumor is resectable but with an involved CRM), decision rules 304-3 (chemotherapy followed by long-course chemoradiotherapy (CRT)) and 304-4 (short course radiotherapy (RT) or long-course CRT followed by chemotherapy) is included in the subset of decision rules 118.

Edge 306-4 of decision tree 302 specifies that the data in the data repository 106 is to include an indication that the tumor is unresectable. If this is the case (e.g., the data repository 106 includes documentation that the tumor is in fact unresectable), decision rule 304-5 (systemic chemotherapy) is included in the subset of decision rules. Edge 306-5 specifies that the data in the data repository 106 is to include an indication that the systemic chemotherapy of node 304-5 has been completed. If this is the case (e.g., the data repository 106 includes documentation that the systemic chemotherapy of node 304-5 has been completed), decision rule 304-6 (resectable) is included in the subset of decision rules. Node 304-6 is associated with a diagnostic procedure to determine whether the tumor is resectable upon completion of the systemic chemotherapy of node 304-5. Edge 306-6 specifies that if the data repository 106 includes information that the tumor is indeed now resectable after completion of the systemic chemotherapy of node 304-5, node 304-7 (short-course RT or long-course CRT) is to be included in the subset of decision rules. Edge 306-7 specifies that if the data repository 106 includes information that the tumor is not resectable after the resectability assay of node 304-6, node 304-8 (systemic chemotherapy) is to be included in the subset of decision rules.

As such, as the above example describes, in some embodiments, the instructions for discovering the first subset of decision rules from among the plurality of decision rules through a modeling procedure that aligns a first condition in the one or more first conditions against the plurality of decision rules comprises traversing the directed graph beginning at an initial node in the directed graph.

Block 222. Referring to block 222, in some embodiments, the plurality of decisions rules is in the form of a decision tree comprising a plurality of decisions, in which each respective decision in the plurality of decisions dictates whether or not to include a decision rule in the plurality of decision rules in the first subset of decision rules based on satisfying or failing to satisfy one or more conditions using data in the data repository. In some such embodiments, the instruction for discovering the first subset of decision rules from among the plurality of decision rules through a modeling procedure that aligns a first condition in the one or more first conditions against the plurality of decision rules comprises traversing the decision tree beginning at an initial decision in the decision tree.

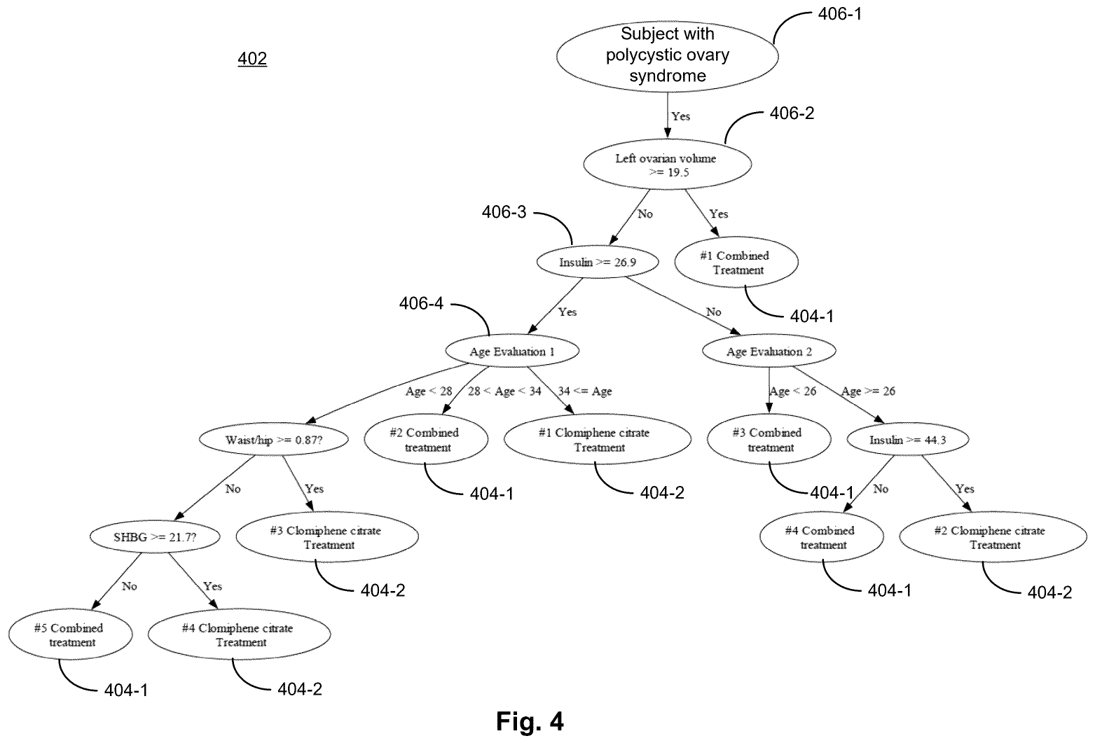

FIG. 4 illustrates. FIG. 4 is a decision tree 402 of a treatment plan for woman with polycystic ovary syndrome. The treatment plan illustrated in FIG. 4 is modeled from a standard of care for this diagnosis described in Zhang et al., 2010, “Decision trees for identifying predictors of treatment effectiveness in clinical trials and its application to ovulation in a study of women with polycystic ovary syndrome,” Human Reproduction 25(10, pp. 2612-2521, which is hereby incorporated by reference. In FIG. 4, the treatment plan has been encoded as a series of decision rules in the form of decision tree 402. Decision tree 402 includes a plurality of decisions 404, in which each respective decision in the plurality of decisions dictates whether or not to include a decision rule in the plurality of decision rules in the first subset of decision rules based on satisfying or failing to satisfy one or more conditions using data in the data repository. The decision tree culminates in two possible decision rules, combined treatment 404-1 and clomiphene citrate treatment 404-2. Combined treatment 404-1 is a decision rule associated with a regimen of clomiphene citrate (CC) and metformin, whereas clomiphene citrate treatment decision rule 404-2 is a decision rule for clomiphene citrate treatment without metformin. The plurality of decisions rules 406 is used to decide which of these decision rule is included in the subset of decision rules. For example, if the subject has polycystic ovary syndrome (406-1—Yes) and the left ovarian volume is greater than, or equal to, 19.5 (406-2—Yes), the decision rule 404-1 (combined treatment) is included in the subset of decision rules. As another example, if the subject has polycystic ovary syndrome (406-1—Yes), the left ovarian volume is less than 19.5 (406-2—No), has an insulin level that is greater than 26.9 (406-3—Yes), and is greater than 34 years of age per decision 406-4, the decision rule 404-2 (clomiphene citrate treatment) is included in the subset of decision rules

As such, as the above example describes, in some embodiments, the instructions for discovering the first subset of decision rules from among the plurality of decision rules through a modeling procedure that aligns a first condition in the one or more first conditions against the plurality of decision rules by traversing a decision tree beginning at an initial decision in the decision tree.

Block 224. Referring to block 224, the plurality of processor executable instructions further comprises instructions for activating, using an evaluation module 122, a first decision rule 118 in the first subset of decision rules whose corresponding triggering condition 120 arises in the data repository 106.

For example, referring to FIG. 3, in some embodiments, in accordance with block 224, decision rule 304-2 (chemotherapy followed by a short course RT or long-course CRT) is activated. In this example, the triggering condition is (i) the inclusion of decision rule 304-2 in the subset of decision rules (because data repository 106 indicated the subject had rectal cancer with synchronous liver/lung metastasis 304-1 and that the tumor was resectable with clear CRM 306-1) and (ii) an absence of any indication in the data repository 106 that the subject had undergone chemotherapy.

As another example, referring to FIG. 4, in some embodiments, in accordance with block 224, decision rule 404-2 (combined treatment) is activated. In this example, the triggering condition is (i) the inclusion of decision rule 404-1 in the subset of decision rules (because data repository 106 indicated the subject has polycystic ovary syndrome (406-1—Yes) and that the left ovarian volume is greater than or equal to 19.5 (406-2—Yes) and (ii) an absence of any indication in the data repository 106 that the subject had undergone combined treatment.

While the above examples with reference to FIGS. 3 and 4 describe the triggering condition of a missing therapy, decision rules 118 can have a number of different types of triggering conditions as described in further detail below on conjunction with blocks 226 through 242.

Block 226. Referring to block 226, in some embodiments, the corresponding triggering condition 120 for the first decision rule 118 arises in the data repository 106 when the data repository is missing a diagnostic test result specified by the first decision rule.

Examples of diagnostic test results include blood test results such as a complete blood count (CBC), blood chemistry panel, and/or lipid profile. CBC measures various components of blood, including red blood cells, white blood cells, hemoglobin, hematocrit, and platelets. CBC test results are typically given in numerical values with reference ranges for comparison. A blood chemistry panel includes tests like glucose, calcium, electrolytes (e.g., sodium, potassium, chloride, bicarbonate), kidney function (BUN, creatinine), and liver function (AST, ALT, ALP, bilirubin). Blood chemistry panel test results are numerical values with reference ranges. A lipid profile measures cholesterol levels, including total cholesterol, LDL (bad cholesterol), HDL (good cholesterol), and triglycerides. Lipid profile results are numerical values with reference ranges.

Examples of diagnostic test results also include urinalysis test results. Urinalysis analyzes the content of urine to check for substances such as glucose, protein, red and white blood cells, and signs of infection. Urinalysis test results can include descriptive terms (e.g., “clear,” “cloudy,” etc.) and numerical values.

Examples of diagnostic tests further include imaging test results such as X-rays, magnetic resonance imaging (MRI), and/or computed tomography (CT) scans. X-rays provide images of bones and certain tissues. X-ray results include descriptions of any abnormalities, fractures, or changes in tissue density. MRI produces detailed images of organs and tissues. MRI results include descriptive findings regarding the presence of tumors, inflammation, or other abnormalities. CT scans provide cross-sectional images of the body. CT scan results include descriptions of any abnormal growths, bleeding, or blockages.

Examples of diagnostic tests results further include electrocardiograms (ECG or EKG) test results. Electrocardiograms measure the electrical activity of the heart. Electrocardiogram results are typically in the form of wave patterns with interpretations indicating normal rhythm, arrhythmias, or other heart conditions.

Examples of diagnostic test results further include biopsies. Biopsies involve the removal of tissue samples for examination. Biopsy results include histopathological descriptions indicating normal tissue, benign conditions, or malignancies (cancer).

Examples of diagnostic test results further include genetic testing. Genetic testing analyzes DNA (e.g., from a biopsy) to identify mutations or variations associated with specific diseases. Genetic testing results indicate the presence or absence of particular genetic markers.

Examples of diagnostic test results further include microbiological tests such as culture tests and polymerase chain reaction (PCR) tests. Culture tests identify bacteria, viruses, or fungi in samples (e.g., blood, urine, sputum, etc.). Culture test results include the type of microorganism found and its sensitivity to antibiotics. PCR detects genetic material of pathogens. PCR test results are usually positive or negative for the presence of specific pathogens.

Examples of diagnostic test results further include serological tests. Serological tests detect antibodies or antigens in the blood. Serological test results indicate the presence of infection or immunity to diseases like HIV, hepatitis, COVID-19, etc.

Block 228. Referring to block 228, in some embodiments, the corresponding triggering condition 120 for the first decision rule 118 arises in the data repository 106 when the data repository includes a diagnostic test result that satisfies a diagnostic test result threshold. In some embodiments, the diagnostic test result threshold is a particular value. For example, in some embodiment the decision rule 404-1 (#1 combined treatment) is triggered when there is no indication of combined treatment in the data repository 106 and the left ovarian volume is greater than or equal to 19.5. In this example, the value 19.5 is a diagnostic test result threshold. As another example, in some embodiment the decision rule 404-2 (#2 clomiphene citrate treatment) is triggered when there is no indication of combined treatment in the data repository 106 and the insulin level of the subject is greater than 44.3. In this example, the value 44.3 is a diagnostic test result threshold.

Block 230. Referring to block 230, in some embodiments, the plurality of instructions further comprises instructions for updating the data repository 106 using multimodal treatment data for the subject, and the corresponding triggering condition 120 for the first decision rule 118 comprises an absence of an evaluation of the subject in the multimodal treatment data for the subject during a predetermined time period associated with the first decision rule.

For example, in some embodiments, blood tests are used to monitor for a chronic condition in subjects that have such conditions or are risk of incurring such conditions. Recurring blood tests for diabetes (HbA1c), cholesterol levels, kidney function (creatinine, BUN), and/or liver function (AST, ALT) are needed for such subjects. Failure to find such test results in the data repository 106 during designated time frames (e.g., every month, every 3 months, every six months, annually, past month, past 3 months, past 6 months, past year, etc.) can be a triggering condition for a decision rule in some embodiments.

As another example, in some embodiments, subjects undergoing anticoagulation therapy and using anticoagulants, like warfarin, need regular INR (International Normalized Ratio) tests to adjust medication dosage. Failure to find INR test results in the data repository 106 for such subjects during designated time frames (e.g., every month, every 3 months, every six months, annually, past month, past 3 months, past 6 months, past year, etc.) can be a triggering condition for a decision rule in some embodiments.

As still another example, for cancer subjects, cancer surveillance in the form of periodic imaging studies like CT scans, MRIs, or mammograms are needed to monitor for recurrence or progression. Failure to find such imaging test results in the data repository 106 for such subjects during designated time frames (e.g., every month, every 3 months, every six months, annually, past month, past 3 months, past 6 months, past year, etc.) can be a triggering condition for a decision rule in some embodiments.

For subjects with chronic conditions, regular imaging for conditions like COPD (chest X-rays), arthritis (joint X-rays), or cardiovascular diseases (echocardiograms), is needed. Failure to find such imaging test results in the data repository 106 for such subjects during designated time frames (e.g., every month, every 3 months, every six months, annually, past month, past 3 months, past 6 months, past year, etc.) can be a triggering condition for a decision rule in some embodiments.

For subjects with a history of colorectal cancer or polyps, gastrointestinal monitoring through periodic colonoscopies and endoscopies are needed. Failure to find such gastrointestinal monitoring results in the data repository 106 for such subjects during designated time frames (e.g., every month, every 3 months, every six months, annually, past month, past 3 months, past 6 months, past year, etc.) can be a triggering condition for a decision rule in some embodiments.

For subjects with end-stage renal disease, hemodialysis or peritoneal dialysis is required multiple times a week. Failure to find a record in the data repository 106 of such dialysis being performed for such subjects during designated time frames (e.g., each day, each two day period, each three day period, past day, past 48 hours, past 72 hours, etc.) can be a triggering condition for a decision rule in some embodiments.

For oral health maintenance, regular dental cleanings and check-ups, typically every six months, and periodic procedures like fillings, crowns, or root canals as needed, are required. Failure to find a record of such dental maintenance in the data repository 106 during designated time frames (e.g., every six months, annually, past 6 months, past year, etc.) can be a triggering condition for a decision rule in some embodiments.

In some embodiments, a triggering condition is an absence of a periodic vaccination such as the flu shot (annually), tetanus booster (every 10 years), or other age- or condition-specific vaccines.

In some embodiments, a triggering condition is an absence an annual or biannual physical examination. Such exams monitor overall health, screen for new conditions, and manage ongoing health issues. Failure to find a record of such physical examinations in the data repository 106 during designated time frames (e.g., every six months, annually, past 6 months, past year, etc.) can be a triggering condition for a decision rule in some embodiments.

In some embodiments, a triggering condition is an absence of an eye exam. Periodic eye exams for individuals with conditions like diabetes (annual dilated eye exams), glaucoma, or macular degeneration, are important to ensure that subjects are receiving appropriate medications for such conditions. Failure to find a record of such eye examinations in the data repository 106 during designated time frames (e.g., every six months, annually, past 6 months, past year, etc.) can be a triggering condition for a decision rule in some embodiments.

For subjects that have incurred hearing loss, periodic audiograms, especially if using hearing aids, is needed to ensure that such subjects are receiving appropriate hearing correction. Failure to find a record of such audiograms in the data repository 106 during designated time frames (e.g., every six months, annually, past 6 months, past year, etc.) can be a triggering condition for a decision rule in some embodiments.

For subjects with pacemakers or implantable cardioverter-defibrillators (ICDs), regular follow-up appointments to check and adjust such devices are needed. Failure to find evidence of such follow-up appointments in the data repository 106 during designated time frames (e.g., every 3 months, every six months, annually, past month, past 3 months, past 6 months, past year, etc.) can be a triggering condition for a decision rule in some embodiments.

For subjects with coronary artery disease, periodic stress tests to monitor heart function and detect ischemia are needed. Failure to find evidence of such periodic stress tests in the data repository 106 during designated time frames for such subjects (e.g., every 3 months, every six months, annually, past month, past 3 months, past 6 months, past year, etc.) can be a triggering condition for a decision rule in some embodiments.

For subjects with asthma, COPD, or other chronic lung diseases, periodic pulmonary function tests such as regular spirometry tests are needed. Failure to find evidence of such periodic tests in the data repository 106 during designated time frames (e.g., every 3 months, every six months, annually, past 3 months, past 6 months, past year, etc.) can be a triggering condition for a decision rule in some embodiments.

For subjects in need of physical therapy, ongoing physical therapy sessions for rehabilitation or management of chronic musculoskeletal conditions are needed. Failure to find evidence of such physical therapy tests in the data repository 106 during designated time frames (e.g., every week, every other week, monthly, past week, past two weeks, past month, etc.) can be a triggering condition for a decision rule in some embodiments.

For subjects with multiple sclerosis (e.g., disease-modifying therapies), rheumatoid arthritis (biologics), or iron-deficiency anemia, periodic intravenous infusions are needed. Failure to find evidence of such infusions in the data repository 106 during designated time frames (e.g., every week, every other week, monthly, past week, past two weeks, past month, etc.) can be a triggering condition for a decision rule in some embodiments.

For subjects with a history of skin cancer or high risk of developing skin cancer, need regular skin checks. Failure to find evidence of such skin checks in the data repository 106 during designated time frames (e.g., every week, every other week, monthly, past week, past two weeks, past month, etc.) can be a triggering condition for a decision rule in some embodiments.

Block 232. Referring to block 232, in some embodiments, the corresponding triggering condition 120 for the first decision rule 118 comprises an indication in the data repository 106 that the subject has been prescribed a combination of medications having a documented adverse pharmacodynamic or pharmacokinetic interaction with each other. In some embodiments, the data repository 106 for a subject indicates that the subject is taking two or more drugs (e.g. a drug combination) concurrently. However, inappropriate drug combination may not only fail to improve a subject's condition. It may even lead to adverse reactions. Therefore, in some embodiments various decision rules are triggered when the data repository indicates that the subject is taking certain combinations of drugs. The risk of harmful drug-drug interactions (DDIs) increases as patients take more kinds of drugs. For example, more than one-third of older Americans regularly use five or more drugs or supplements, and 15% are at risk for serious DDIs. See Zhao, 2024, “Drug-drug interaction prediction: databases, web servers and computational models,” Briefings in Bioinformatics 25(1), pp. 1-28, which is hereby incorporated by reference. In some embodiments, drug-drug interactions are found in DrugBank (Wishart et al., 2018, “DrugBank 5.0: a major update to the DrugBank database for 2018,” Nucleic Acids Res 46: D1074-82), DDInter (Xiong et al., 2022, “DDInter: an online drug-drug interaction database towards improving clinical decision-making and patient safety,” Nucleic Acids Res 50: D1200-d1207), SuperDRUG2 (Siramshetty et al., 2018, “SuperDRUG2: a one stop resource for approved/marketed drugs,” Nucleic Acids Res 46: D1137-d1143), INXBASE (Bottiger et al., 2009, “SFINX-a drug-drug interaction database designed for clinical decision support systems,” Eur J Clin Pharmacol 65:627-33), OncoRx (Yap et al., 2010, “An onco-informatics database for anticancer drug interactions with complementary and alternative medicines used in cancer treatment and supportive care: an overview of the OncoRx project,” Support Care Cancer 18:883-91), the Drug Interaction Database (DIDB) (Hachad et al., 2010, “A useful tool for drug interaction evaluation: the University of Washington Metabolism and Transport Drug Interaction Database. Hum Genomics 5, pp. 61-72); DrugComb (Zheng et al., 2021, “DrugComb update: a more comprehensive drug sensitivity data repository and analysis portal,” Nucleic Acids Res 49: W174-84); and DailyMed (United States National Library of Medicine, 8600 Rockville Pike, Bethesda, Maryland, 20894). Such databases include information about known adverse drug-drug interactions.

In some embodiments, a triggering condition for a first decision rule 118 comprises an indication in the data repository 106 that the subject is concurrently taking warfarin and a non-steroidal anti-inflammatory drug (NSAID). Such a combination can result in increased risk of bleeding. Warfarin is an anticoagulant, and NSAIDs can inhibit platelet function and cause gastrointestinal bleeding. Together, they significantly raise the risk of serious bleeding.

In some embodiments, a triggering condition for a first decision rule 118 comprises an indication in the data repository 106 that the subject is concurrently taking an ACE inhibitor and a potassium-sparing diuretics. Such a combination can result in hyperkalemia (high potassium levels). ACE inhibitors (e.g., lisinopril) decrease aldosterone, leading to increased potassium levels. Potassium-sparing diuretics (e.g., spironolactone) further increase potassium retention, risking dangerous hyperkalemia.

In some embodiments, a triggering condition for a first decision rule 118 comprises an indication in the data repository 106 that the subject is concurrently taking a statin and a macrolide antibiotic. Such a combination can result in increased risk of myopathy or rhabdomyolysis. Statins (e.g., simvastatin) are metabolized by the CYP3A4 enzyme. Macrolides (e.g., erythromycin, clarithromycin) inhibit CYP3A4, increasing statin levels and the risk of muscle damage.

In some embodiments, a triggering condition for a first decision rule 118 comprises an indication in the data repository 106 that the subject is concurrently taking a selective serotonin reuptake inhibitors (SSRI, e.g., fluoxetine) and a monoamine oxidase inhibitor (MAOIs, e.g., phenelzine). SSRIs and MAOIs both increase serotonin levels. Their combination can lead to high serotonin levels, causing serotonin syndrome. Such a combination can result in serotonin syndrome.

In some embodiments, a triggering condition for a first decision rule 118 comprises an indication in the data repository 106 that the subject is concurrently taking digoxin and amiodarone. Such a combination can result in increased risk of digoxin toxicity. Amiodarone increases the blood levels of digoxin by inhibiting its clearance, raising the risk of digoxin toxicity, which can cause arrhythmias and other serious side effects.

In some embodiments, a triggering condition for a first decision rule 118 comprises an indication in the data repository 106 that the subject is concurrently taking a benzodiazepine and an opioid. Such a combination can result in increased risk of severe respiratory depression and sedation. Both benzodiazepines (e.g., diazepam) and opioids (e.g., oxycodone) depress the central nervous system. When combined, they can significantly increase the risk of life-threatening respiratory depression.

In some embodiments, a triggering condition for a first decision rule 118 comprises an indication in the data repository 106 that the subject is concurrently taking metronidazole and consuming alcohol. Such a combination can result in a disulfiram-like reaction. Metronidazole can inhibit the metabolism of alcohol, leading to a build-up of acetaldehyde. This causes symptoms similar to those of disulfiram (Antabuse), including nausea, vomiting, flushing, and palpitations.

In some embodiments a triggering condition for a first decision rule 118 comprises an indication in the data repository 106 that the subject is concurrently taking a calcium channel blocker and a beta-blocker. Such a combination can result in increased risk of bradycardia and heart block. Both drug classes decrease heart rate and contractility. Together, together they can cause severe bradycardia, hypotension, and heart block.

In some embodiments, a triggering condition for a first decision rule 118 comprises an indication in the data repository 106 that the subject is concurrently taking an anticoagulants and an antiplatelet agent. Such a combination can result in increased risk of bleeding.

In some embodiments, a triggering condition for a first decision rule 118 comprises an indication in the data repository 106 that the subject is concurrently taking an anticoagulants (e.g., heparin, warfarin, etc.) and an antiplatelet agent (e.g., aspirin, clopidogrel, etc.). Both inhibit blood clotting through different mechanisms, leading to an additive effect and increased bleeding risk.

In some embodiments, a triggering condition for a first decision rule 118 comprises an indication in the data repository 106 that the subject is concurrently taking phenytoin and an oral contraceptive. Such a combination can result in reduced effectiveness of the oral contraceptive. Phenytoin induces hepatic enzymes that increase the metabolism of oral contraceptives, reducing their efficacy and increasing the risk of unintended pregnancy.

Block 234. Referring to block 234, in some embodiments, the corresponding triggering condition 120 for the first decision rule 118 comprises an absence of an indication in the data repository 106 of a clinical trial for which the subject is currently eligible. For instance, consider the case in which the data repository 106 indicates that the subject is an adult female, between the ages of 18 and 64, with endometriosis, that lives in Mobile Alabama. In this instance, a decision rule may be activated when it is further noted that there is no indication in the data repository 106 that the subject has not considered enrolment in clinical trial NCT05862272, which is a phase 3B study to evaluate bone mineral density with long-term use of Relugolix combination table in women with endometriosis. Available clinical trials are found at clinicaltrials.gov. In some embodiments, decision rules are built around all the qualifying criteria of each of the clinical trials currently enrolling subjects. In such instances, the qualifying criteria of a particular clinical trial are the triggering conditions 120 for a particular decision rule. In some embodiments, when the data repository 106 indicates that the subject has the qualifying criteria for a clinical trial and the subject is not enrolled in the clinical trial, a corresponding decision rule 118 is activated for the purposes of notifying one or more contacts 112 that the subject is eligible for the clinical trial.

Block 236. Referring to block 236, in some embodiments, the corresponding triggering condition 120 for the first decision rule 118 comprises an absence of a therapy in the data repository 106. This decision rule is similar to the decision rules described above in conjunction with block 230 and a number of the decision rules described in block 230 are applicable for block 236.

An additional example of a decision rule in accordance with block 236 is a corresponding triggering condition for a decision rule that is triggered when the data repository 106 indicates that the subject presently has a bacterial infection but there is no indication in the data repository 106 that the subject is taking an antibiotic.

Another example is a corresponding triggering condition for a decision rule that is triggered when the data repository 106 indicates that the subject presently has cancer but there is no indication in the data repository 106 that the subject is taking chemotherapy.

Another example is a corresponding triggering condition for a decision rule that is triggered when the data repository 106 indicates that the subject has hypertension but there is no indication in the data repository 106 that the subject is taking an antihypertensive to manage high blood pressure (e.g., lisinopril, amlodipine, etc.)

Another example is a corresponding triggering condition for a decision rule that is triggered when the data repository 106 indicates that the subject has diabetes but there is no indication in the data repository 106 that the subject is on insulin therapy to regulate blood sugar levels.

Another example is a corresponding triggering condition for a decision rule that is triggered when the data repository 106 indicates that the subject smokes but there is no indication in the data repository 106 that the subject has been provided information about smoking cessation programs.

Block 238. Referring to block 238, in some embodiments, the corresponding triggering condition 120 for the first decision rule 118 arises in the data repository 106 when the data repository is missing a medical procedure. This decision rule is similar to the decision rules described above in conjunction with block 230 and a number of the decision rules described in block 230 are applicable for block 236.

An additional example of a decision rule in accordance with block 238 is a corresponding triggering condition for a decision rule that is triggered when the data repository 106 indicates that the subject presently has appendicitis but there is no indication in the data repository 106 that the subject has had an appendectomy.

Another example is a corresponding triggering condition for a decision rule that is triggered when the data repository 106 indicates that the subject has coronary heart disease but there is no indication in the data repository 106 that the subject has received angioplasty or stenting.

Another example is a corresponding triggering condition for a decision rule that is triggered when the data repository 106 indicates that the subject has a gallstone but there is no indication in the data repository 106 that the subject has received a laparoscopic cholecystectomy to remove the gallstone.

Another example is a corresponding triggering condition for a decision rule that is triggered when the data repository 106 indicates that the subject has chronic sinusitis but there is no indication in the data repository 106 that the subject has received endoscopic sinus surgery to remove blockages in the sinuses.

Another example is a corresponding triggering condition for a decision rule that is triggered when the data repository 106 indicates that the subject has a gastrointestinal disorder but there is no indication in the data repository 106 that the subject has received an endoscopy to examine the digestive tract.

Another example is a corresponding triggering condition for a decision rule that is triggered when the data repository 106 indicates that the subject has incurred significant blood loss but there is no indication in the data repository 106 that the subject has received a blood transfusion to replace the lost blood.

Another example is a corresponding triggering condition for a decision rule that is triggered when the data repository 106 indicates that the subject has arrhythmias but there is no indication in the data repository 106 that the subject has a pacemaker implantation.

Another example is a corresponding triggering condition for a decision rule that is triggered when the data repository 106 indicates that the subject has leukemia, lymphoma, or other blood disorders but there is no indication in the data repository 106 that the subject has received a bone marrow transplant.

Another example is a corresponding triggering condition for a decision rule that is triggered when the data repository 106 indicates that the subject is morbidly obese but there is no indication in the data repository 106 that the subject has undergone gastric bypass surgery.

Another example is a corresponding triggering condition for a decision rule that is triggered when the data repository 106 indicates that the subject has kidney stones but there is no indication in the data repository 106 that the subject has undergone extracorporeal shock wave lithotripsy to break the kidney stones down.

Block 240. Referring to block 240, in some embodiments, the corresponding triggering condition 120 for the first decision rule 118 arises in the data repository 106 when the data repository includes a medical procedure result that specifies the subject has a predetermined condition specified by the corresponding triggering condition of the first decision rule.

In one such example in accordance with block 240, a blood assay indicates that the subject has a fasting blood glucose that is greater than or equal to 126 mg/dl and HbA1c that is greater than 6.5%, indicating that the subject has diabetes.

In another such example in accordance with block 240, the subject is male and a blood assay indicates that the subject has hemoglobin of less than 13.5 g/dL and hematocrit of less than 41%, indicating that the subject is anemic.

In another such example in accordance with block 240, a blood assay indicates that the subject has total cholesterol of greater than or equal to 240 mg/dL, LDL cholesterol of greater than or equal to 160 mg/dL, and HDL cholesterol of less than or equal to 50 mg/dl indicating that the subject has hyperlipidemia.

In another such example in accordance with block 240, a chest x-ray or CT scan indicates that the presence of an irregularly shaped mass or nodule in the lungs, indicating that the subject has lung cancer.

In another such example in accordance with block 240, an x-ray reveals joint space narrowing, osteophytes (bone spurs), subchondral sclerosis, and/or subchondral cysts, indicating that the subject has osteoarthritis.

In another such example in accordance with block 240, an electrocardiogram shows irregular rhythm with no distinct P waves indicating that the subject has undergone an atrial fibrillation.

Block 242. Referring to block 242, in some embodiments, the response to the first condition 120 at the first entity is a first institution based care plan at an institution and the first subset of decision rules model the first institution based care plan for a first diagnosis indicated by the activating of the first decision rule 118/124 as a first decision tree. An example of this is shown in FIG. 3, where the first diagnosis is rectal cancer with synchronous liver/lung metastasis and each of the decision rules in the decision tree 302 are carried out at the same institution in accordance with the decision tree.

Block 243. Referring to block 243, the plurality of processor executable instructions further comprises instructions for identifying a first notification rule 128 in a notification rule repository 126 that is actionable upon the activating of the first decision rule 118/124. Each such notification rule 128 specifies who is to be notified and what the notification should be.

Block 244. Referring to block 244, the plurality of processor executable instructions further comprises instructions for communicating a first notification to a first notification path 130 in accordance with the first notification rule 128, using a computer network. The first notification path consists of a first subset of the plurality of contacts 110.

An example of a first notification path that consists of a first subset of the plurality of contacts 110, consider the case where a subject has been a patient at an institution for a number of years, during which time the subject has had regular physical examinations, treatment for asthma, and hearing tests. As part of a recent annual physical examination, blood work results are ordered indicating that the subject that high cholesterol levels. Months go by since that blood tests are ordered with no indication in the data repository that the subject has been put on cholesterol medications or counseled on change of diet or exercise. In this instance, a decision rule 118 that checks for such a care gap (triggering condition 120) is activated and a notification rule 128 that is actionable because the decision rule 118 has been activated results in the communication of a first notification to a first notification path in accordance with the notification rule. In this example, the message is sent to the subject's primary physician regarding the possible need to counsel the subject on the high cholesterol. The other health workers that have attended the subject over the years, such as the audiologist and allergy specialist are not included in this notification path.

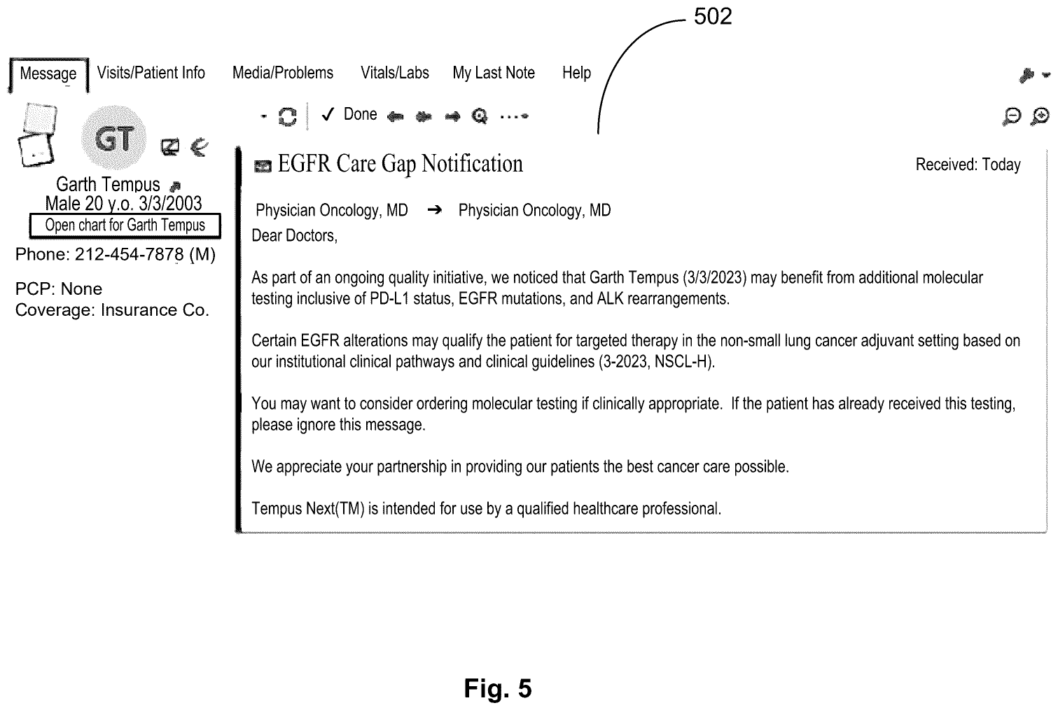

In some embodiments the first notification is an encrypted E-mail, an update to the electronic health record, and/or a message provided in a portal associated with system 100. FIG. 5 illustrate a first notification in accordance with an embodiment of the present disclosure. The notification is directed to the oncology specialist that is treating the subject. In this example the subject's name is “Garth Tempus.” The notification, triggered by the activation of a decision rule 118 and communicated in accordance with a notification rule 128 associated with the activated decision rule, indicates that the subject may benefit from additional molecular testing inclusive of PD-L1 status, EGFR mutations, and ALK rearrangements. The notification further indicates that certain EGFR mutations may qualify the subject for a targeted therapy in a non-small cell lung cancer adjuvant setting.

Block 246. Referring to block 246, in some embodiments, the response to the first condition 110 at the first entity is a first institution based care plan at an institution and the first notification path 130 is an identity of a first contact 112 in the plurality of contacts having a first specialty associated with activation of the first decision rule 118/124. This embodiment can be illustrated using the case of the recent annual physical examination given above in block 244. During the physical examination, the subject complains of allergies and asthma. Allergy tests are ordered. The allergy tests indicate that the allergies are compounding the asthma. Months go by since that allergy tests are ordered with no indication in the data repository that the subject has been advised of the allergy test results and counseled on how to treat the allergies to alleviate the asthma. In this instance, a decision rule 118 that checks for such a care gap (triggering condition 120) is activated and a notification rule 128 that is actionable because the decision rule 118 has been activated results in the communication of a first notification to a first notification path in accordance with the notification rule. In this example, the message is sent to both the subject's primary physician and to the subject's allergist regarding the possible need to counsel the subject on the allergies. The other health workers that have attended the subject over the years, such as the audiologist, are not included in this notification path.

Block 248. Referring to block 248, in some such embodiments, a second notification is communicated to a second contact having the first specialty when the first contact fails to respond to the first notification. For example, in the example of block 246, if the subject's allergist is on vacation or otherwise does not open or otherwise respond to the first notification, in accordance with block 248, a second notification is communicated to a second allergist at the institution so that the subject's allergies and asthma are alleviated.

Block 250. Referring to block 250, in some embodiments, the response to the first condition 110 at the first entity is a first institution based care plan at an institution and the first notification path 130 is an identity of a primary caregiver for the subject, in the plurality of contacts associated with the subject, at the institution. Such an embodiment has been described in the example given in block 244.

Block 252. Referring to block 252, in some embodiments, the response to the first condition 110 at the first entity is a first institution based care plan at an institution and the first notification path 130 is an identity of a caregiver for the subject, in the plurality of contacts associated with the subject, that most recently attended to the subject at the institution.

Block 254. Referring to block 254, in some embodiments, the response to the first condition 110 at the first entity is a first institution based care plan at an institution and the first notification path 130 is an identity of a caregiver for the subject, in the plurality of contacts associated with the subject, that most frequently attends to the subject at the institution.

Block 256. Referring to block 256, in some embodiments, the response to the first condition 110 at the first entity is a first institution based care plan at an institution and the first notification path 130 is an identity of a caregiver for the subject, in the plurality of contacts associated with the subject, that has been assigned responsibility for administering an upcoming medical procedure in accordance with the first institution based care plan.

Block 258. Referring to block 258, in some embodiments, the first notification rule 128 is associated with a first type of caregiver and, when the plurality of contacts associated with the subject includes a first caregiver of the first type, the first notification path includes the first caregiver of the first type, and when the plurality of contacts associated with the subject does not include a first caregiver of the first type, the first notification path 130 includes a primary caregiver, in the plurality of contacts 112 associated with the subject.

This embodiment can be illustrated using the case of the recent annual physical examination given above in block 244. During the physical examination, the subject complains of allergies and asthma. Allergy tests are ordered. The allergy tests indicate that the allergies are compounding the asthma. Months go by since that allergy tests are ordered with no indication in the data repository that the subject has been advised of the allergy test results and counseled on how to treat the allergies to alleviate the asthma. In this instance, a decision rule 118 that checks for such a care gap (triggering condition 120) is activated and a notification rule 128 that is actionable because the decision rule 118 has been activated results in the communication of a first notification to a first notification path in accordance with the notification rule. In this example, the message is sent to the subject's allergist regarding the possible need to counsel the subject on the allergies. However, if the subject does not have an allergist, the notification is sent to the subject's primary caregiver. The other health workers that have attended the subject over the years, such as the audiologist, are not included in this notification path.

In other embodiments in accordance with block 258, the first notification is intended to drive an appropriate caregiver to rectify a care gap, while the second notification is intended to elicit a recommendation for a caregiver who can rectify the care gap.

In other embodiments, the first and second notifications are the same notification.

Block 260. Referring to block 260, in some embodiments, the plurality of processor executable instructions further comprises instructions for firing, using the evaluation module 122, a second decision rule in the first subset of decision rules whose corresponding triggering condition 120 arises in the data repository 106. Moreover a notification associated with the second decision rule is suppressed when the first decision rule is fired. Such an embodiment is useful to prevent excessive notifications from being sent. For instance a high cholesterol result may activate a first decision rule when the subject is not subsequently counseled on the condition, as discussed in the examples above. The high cholesterol result may also activate a second decision rule regarding the high cholesterol itself. The notifications, while different, need not both be sent. Thus, in some embodiments in accordance with block 260, only one of the two notification is sent.

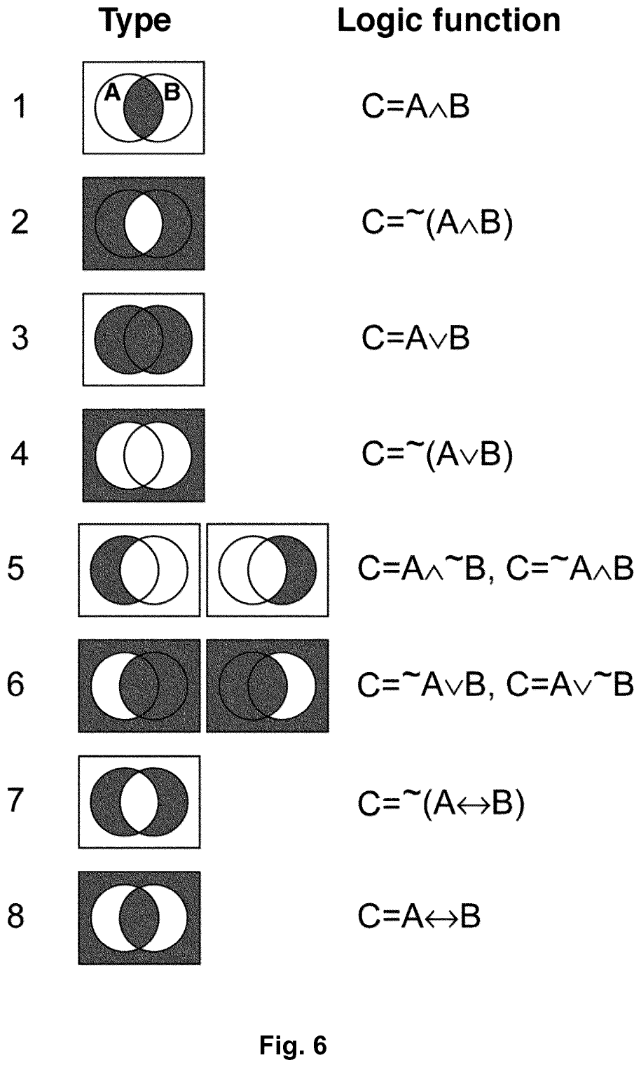

Any form of logic can be applied to decide whether or not certain notifications are communicated based on which decision rules have been activated. In this regard, in some embodiments of the present disclosure, a notification rule may have a more complex relationship with activated decisions rules. FIG. 6 illustrates some examples.

In example 1 of FIG. 6, a first notification is communicated to a first notification path 130 in accordance with a first notification rule 128 if and on if both a first decision rule 118-1 and a second decision rule 118-2 have been activated.

In example 2 of FIG. 6, a first notification is communicated to a first notification path 130 in accordance with a first notification rule 128 if a first decision rule 118-1 has not been activated or a second decision rule 118-2 has not been activated.

In example 3 of FIG. 6, a first notification is communicated to a first notification path 130 in accordance with a first notification rule 128 if a first decision rule 118-1 has been activated or a second decision rule 118-2 has been activated.

In example 4 of FIG. 6, a first notification is communicated to a first notification path 130 in accordance with a first notification rule 128 if both a first decision rule 118-1 and a second decision rule 118-2 have both not been activated.

In example 5 of FIG. 6, a first notification is communicated to a first notification path 130 in accordance with a first notification rule 128 if (i) a first decision rule 118-1 has been activated while a second decision rule 118-2 has not been activated or (ii) the first decision rule 118-1 has not been activated and the second decision rule 118-2 has been activated.

In example 6 of FIG. 6, a first notification is communicated to a first notification path 130 in accordance with a first notification rule 128 if (i) the second decision rule 118-2 has been activated while the first decision rule 118-1 has not been activated or (ii) the second decision rule 118-2 has not been activated and the first decision rule 118-1 has been activated.

In example 7 of FIG. 6, a first notification is communicated to a first notification path 130 in accordance with a first notification rule 128 if one of either the first decision rule 118-1 or the second decision rule 118-2 has been activated.

In example 8 of FIG. 6, a first notification is communicated to a first notification path 130 in accordance with a first notification rule 128 if (i) the first decision rule 118-1 and the second decision rule 118-2 have both been activated or (ii) the first decision rule 118-1 and the second decision rule 118-2 have both not been activated.

Block 262. Referring to block 262, in some embodiments, the plurality of processor executable instructions further comprises instructions for firing, using the evaluation module 122, a second decision rule in the first subset of decision rules whose corresponding triggering condition 120 arises in the data repository 106. A second notification rule 128 in the notification rule repository 126 is identified that is actionable upon the firing of the second decision rule. The second notification rule is a hierarchical notification rule that is dependent upon the firing of the first decision rule and the second decision rule. A second notification is communicated to a second notification path specified by the second notification rule. The second notification path consists of a second subset of the plurality of contacts. This embodiment is similar to example 1 of block with the exception that two notifications are communication, a first one along a first notification path, and a second one along a second notification path.

### III. Examples

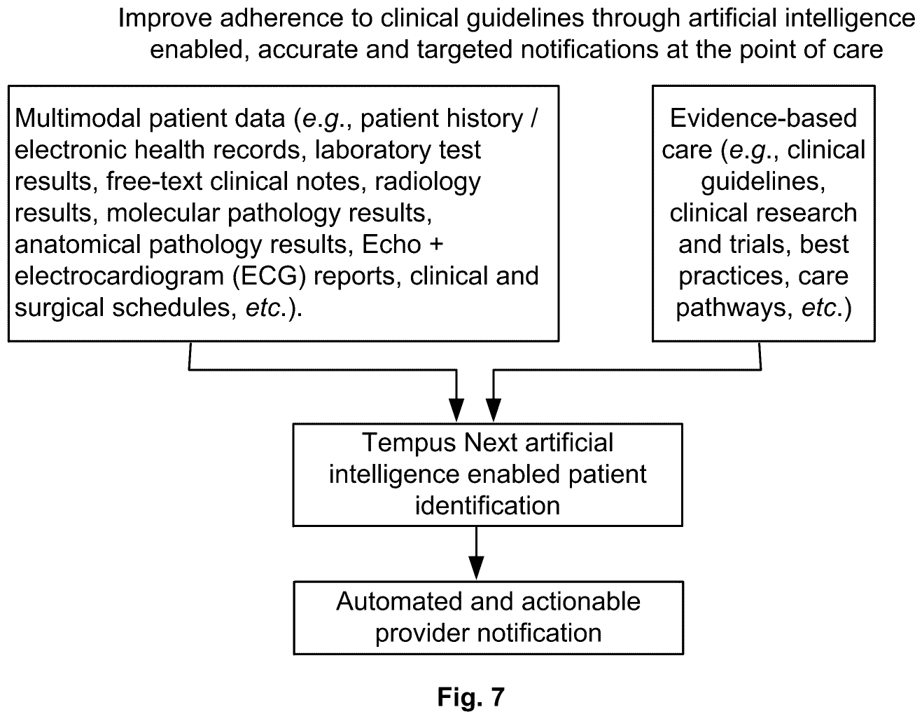

FIGS. 7 through 13 illustrate an example of the present disclosure in the form of Tempus Next, an artificial intelligence enabled care pathway platform that is designed to help medical practitioners spot and address care gaps (triggering conditions 120 of decision rules 118) to accelerate the adoption of evidence-based care.

In this example Tempus Next is used to bridge the genomic testing care gap in early stage non-small cell lung cancer. To this end, Tempus Next plays a key role in notifying clinicians at key moments in the care of early stage non-small cell lung cancer patient so that patients receive the testing they need for precision therapies. As illustrated in FIG. 7, and described in further detail in blocks 202 and 204 above, the present disclosure (e.g., Tempus Next) uses the power of multimodal data and up to date clinical guidelines, to sift through these the multimodal data and guidelines to pinpoint care opportunities (e.g., triggering conditions 120).

When a care gap is detected (e.g., a decision rule 118 is activated because the conditions of its triggering condition 120 have been satisfied), Tempus Next promptly delivers an actionable and automated notification to the clinician bringing the right patient into focus at the right moment for intervention.

In this example, when a project is initiated, a specific subset of patients categorized by ICD-10 codes and other clinical information at each institution where Tempus Next is deployed was identified. In the case of early stage non-small cell lung cancer, across Next projects, thousands of patients have been screened across multiple sites in order to close meaningful patient care gaps in this population. Multimodal clinical data from this group of patients was sent to Tempus for in-depth analysis. In the background, the clinical documents from patients, including both structured and unstructured data, such as surgical notes, radiology and pathology reports, was scanned using artificial intelligence and natural language processing to extract relevant information to identify gaps in care.

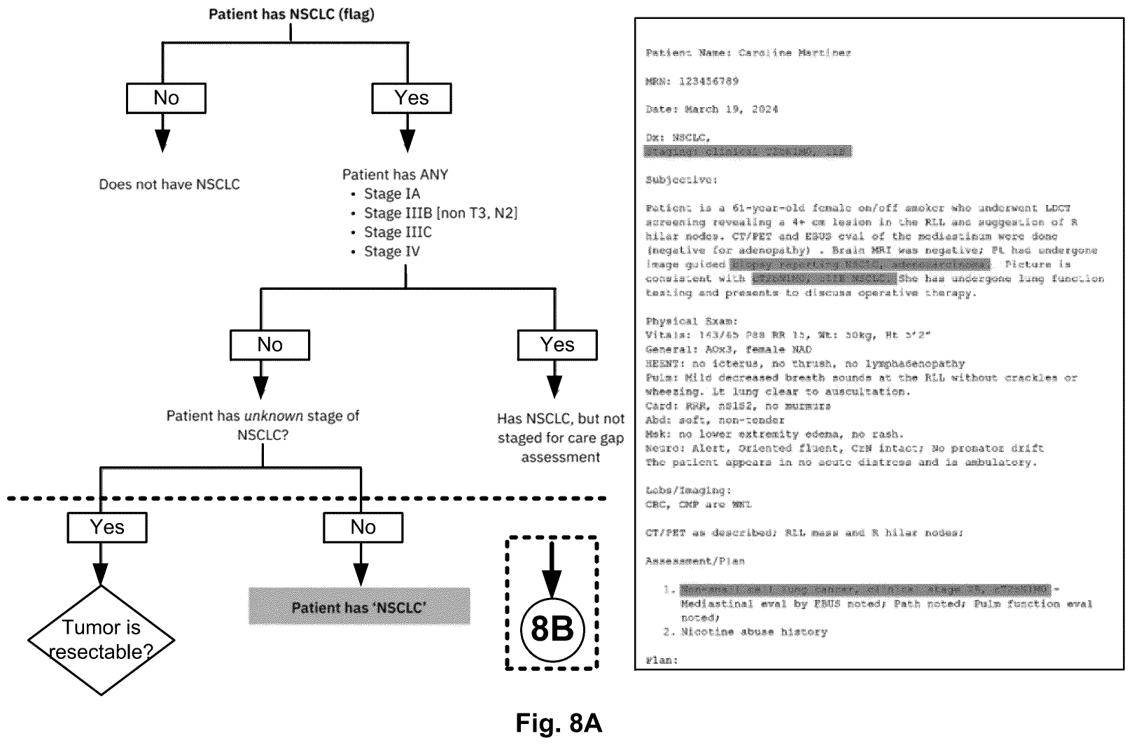

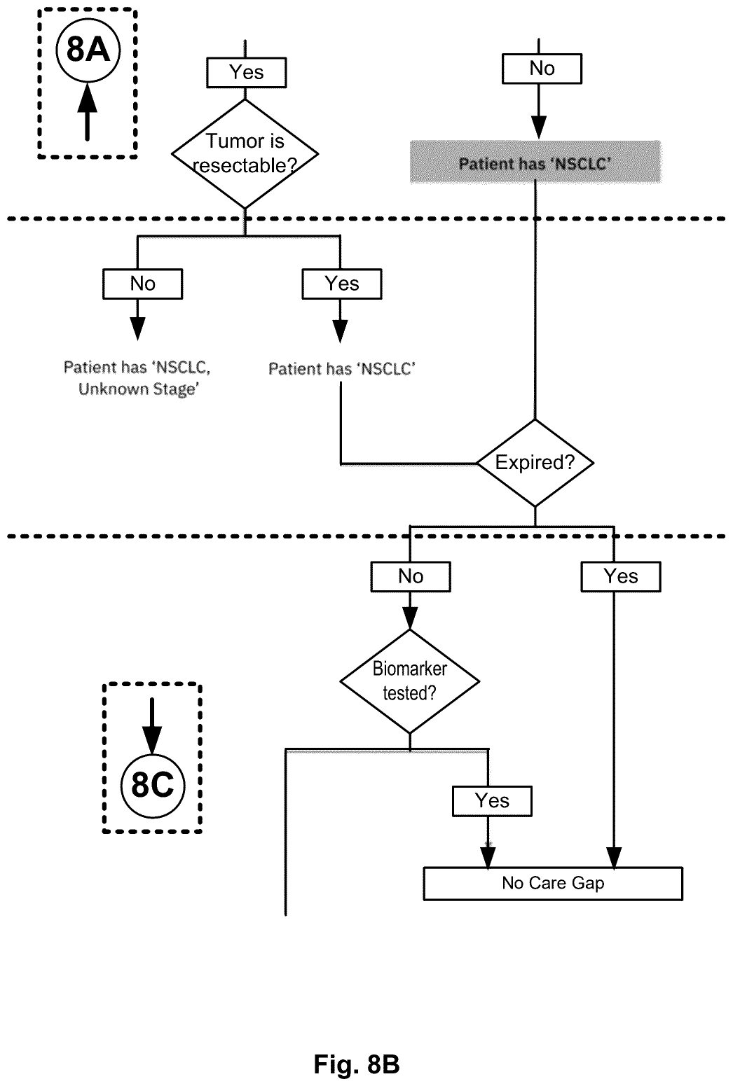

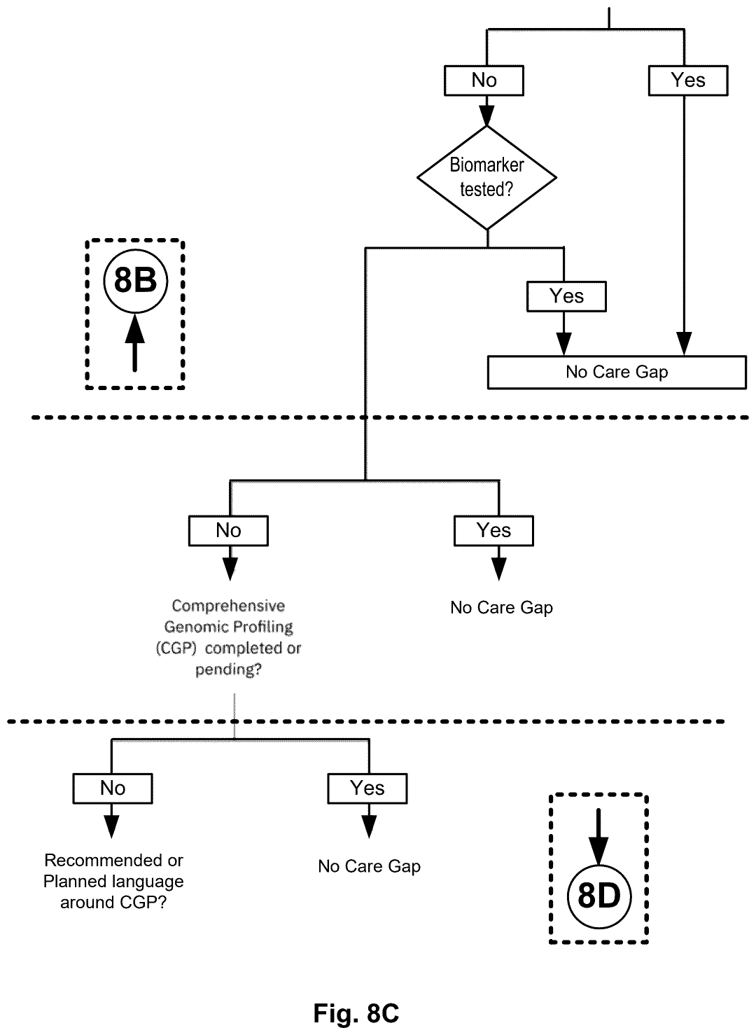

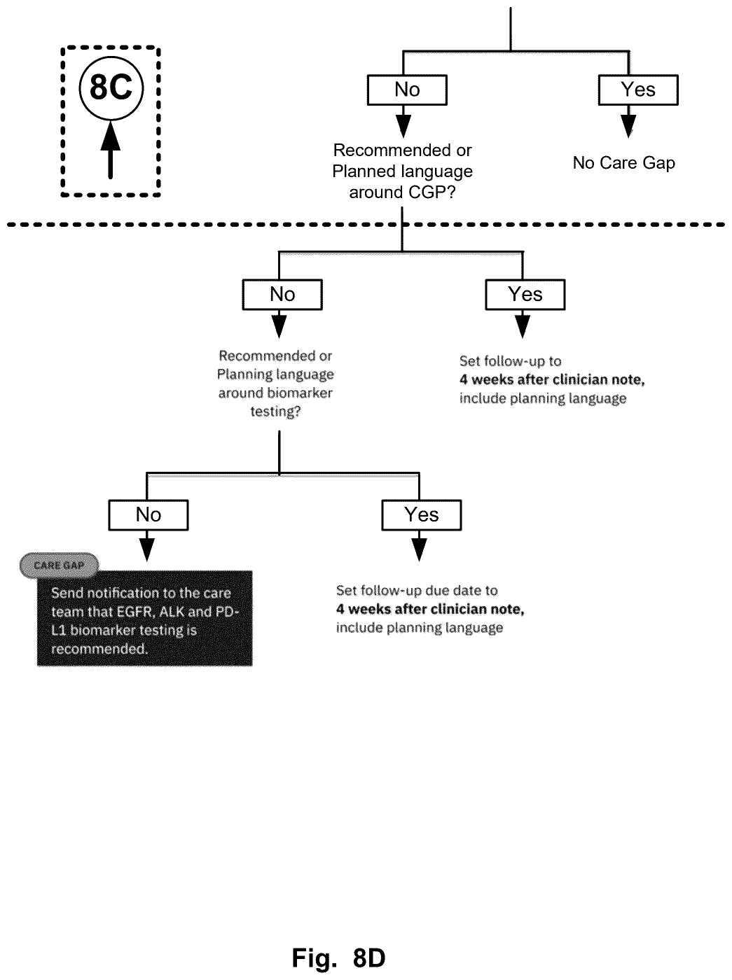

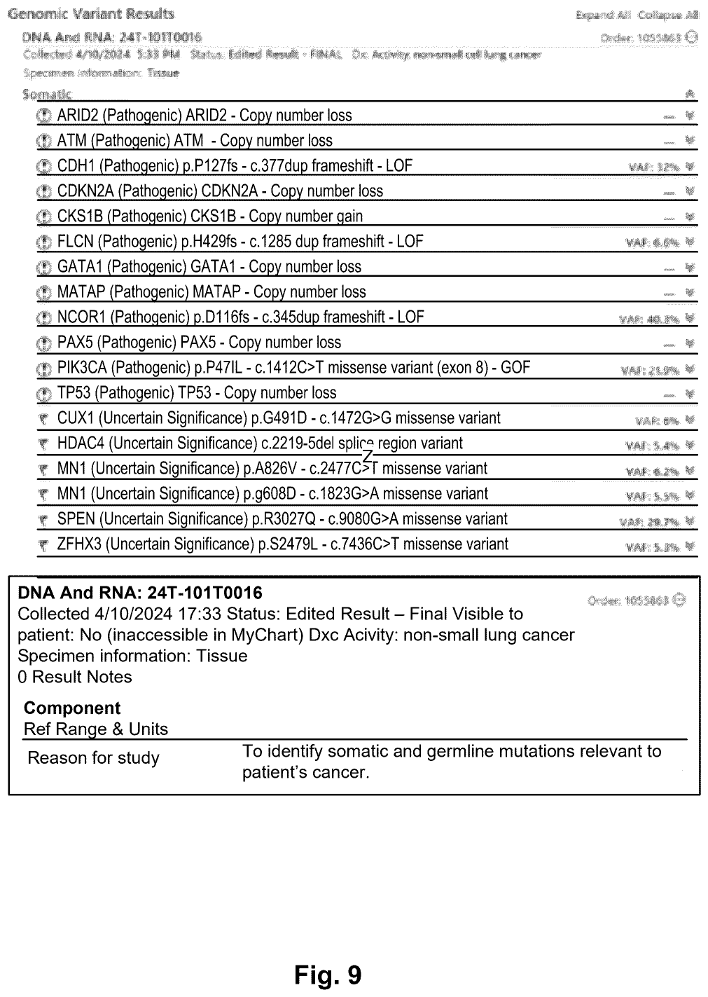

In one such patient, illustrated in FIGS. 8A, 8B, 8C, and 8D, Tempus Next compared relevant clinical information in the data to clinical guidelines (e.g., decision rules 118) and identified that an important biomarker testing had been overlooked for the patient. As illustrated in FIGS. 8A, 8B, 8C, and 8D, there is a care gap (triggering condition 120 of a decision rule 118) identified for EGFR, ALK, and PDLI testing for the patient that was eligible and should have received it based on clinical guidelines. A test was ordered for this patient and the biomarker results were added to the subject's electronic health record (EHR) as illustrated in FIG. 9. With recent updates to clinical guidelines, certain biomarker linked therapies have become available for adjuvant treatment of early stage operable non-small cell lung cancer such as those with EGFR or ALK mutations, a group that historically may not have been biomarker tested. So identifying these care gaps can have significant impact on a patient's care.

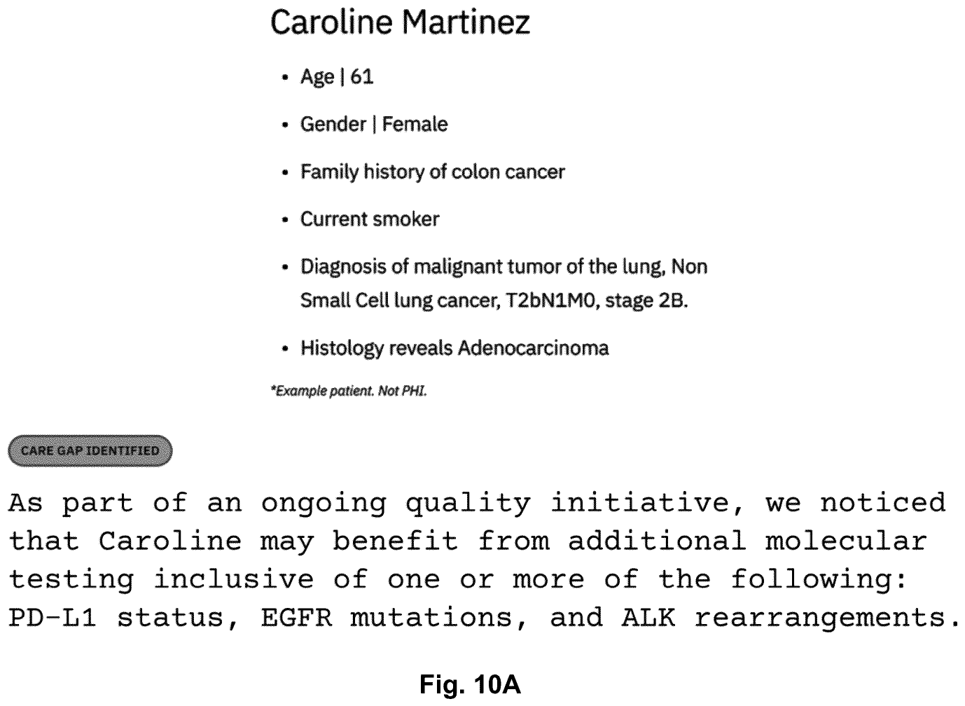

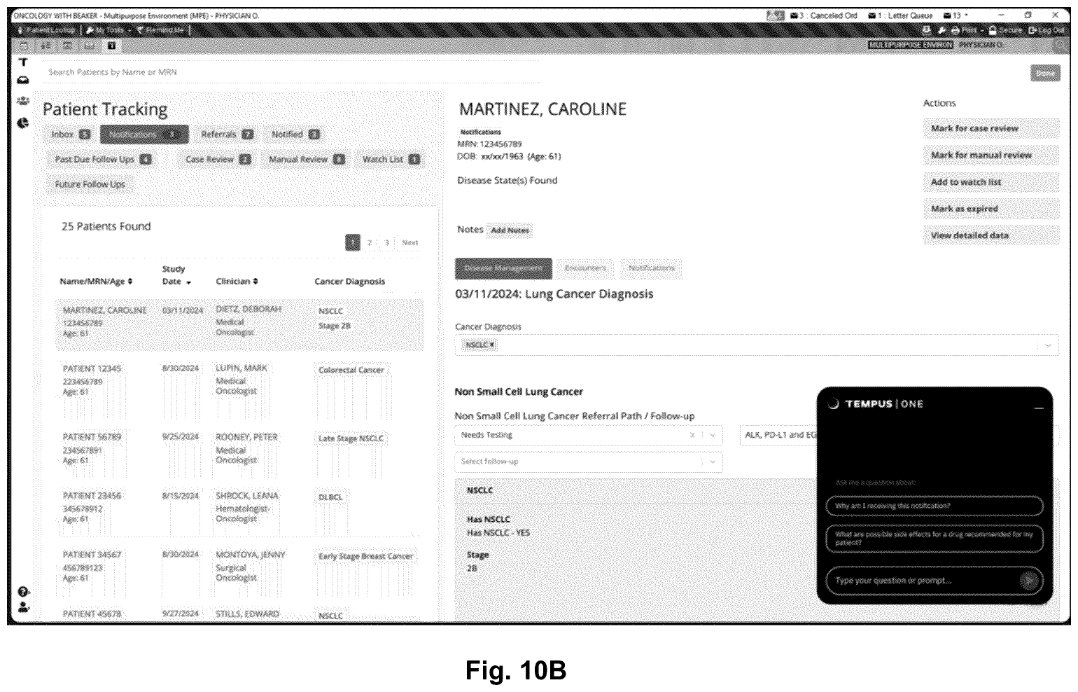

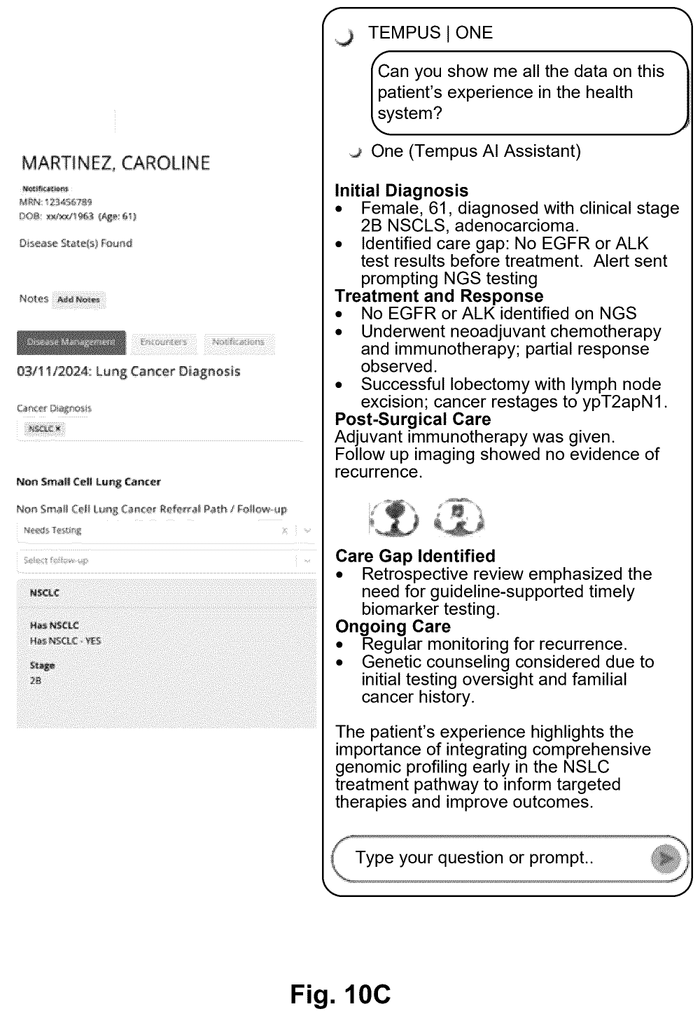

With reference to FIG. 10A, the example continues with, Caroline Martinez, a hypothetical patient who had been diagnosed with resectable clinical stage 2B non-small cell lung cancer. Caroline was eligible for biomarker testing for EGFR, ALK, and PDLI, which would help guide perioperative treatment decisions but had not yet received it. So she was identified by Tempus Next for intervention. Her provider was notified and the provider ordered the appropriate testing for Caroline to help guide perioperative treatment decisions regarding adjuvant targeted therapy or neoadjuvant targeted IO chemo based therapy. When Tempus Next identifies a care gap, such as the missing biomarker tests for Caroline, it promptly notifies the care team with reference to the clinical guideline (e.g. using notification rules 128). For instance, such a notification is illustrated in the bottom portion of FIG. 10A. These notifications put the right data at the care team's fingertips at the right time. They are highly tailored, relevant for the patient and actionable. Moreover, there is flexibility on how to customize how notifications are received, whether through an encrypted email or notification directly within the most commonly used electronic health records, within the Tempus Next portal, or through automated or manual notifications sent to providers or care coordinators enabling the right team member to take relevant action swiftly to get patients care.

Referring to FIG. 10B, Tempus One is an artificial intelligence enabled clinical assistant that can help clinicians that have additional questions about the guideline recommended next steps for their patients. In this case, the clinician wants to know more about Caroline's longitudinal patient journey. Tempus One is asked: Can you show me all the data on this patient's experience in the health system? In response, as illustrated in FIG. 10C, Tempus One is able to query the documents reviewed by Tempus, including the multimodal data such as radiology, imaging, pathology and/or test results in order to provide a chronological history of the patient's journey.

The systems and methods of the present disclosure can be asked questions such as “Why am I receiving this notification?” or “What are possible side effects for a drug recommended for my patient?” The systems and methods of the present disclosure can be asked questions such as “Why was this patient flagged for the care gap?” Thus, as illustrated in FIG. 10, the systems and methods of the present disclosure can deliver on the promise of using technology to help serve patients at the health system level.

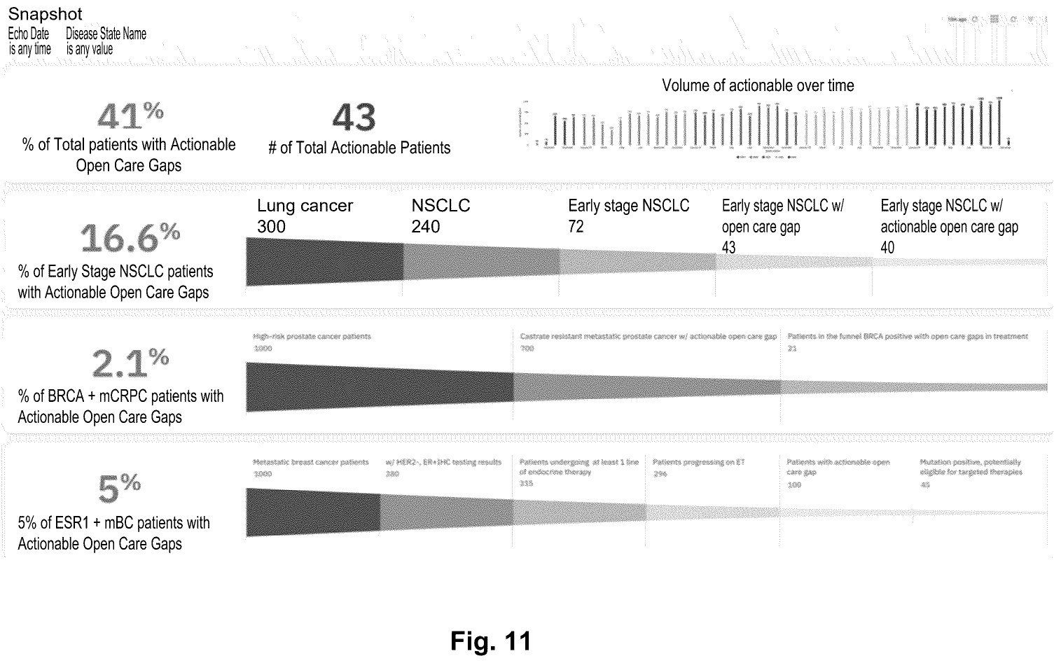

The systems and methods of the present disclosure equip administrators with a streamlined portal, including patient tracking for management of patients with current disease, patient lists to search for subsets of patients, and dashboards to track insights into a hospital patient population. FIG. 11 provides an example of how early stage non-small cell lung cancer might be tracked across the institution, in this case where 16.6% of patients have actionable open care gaps.

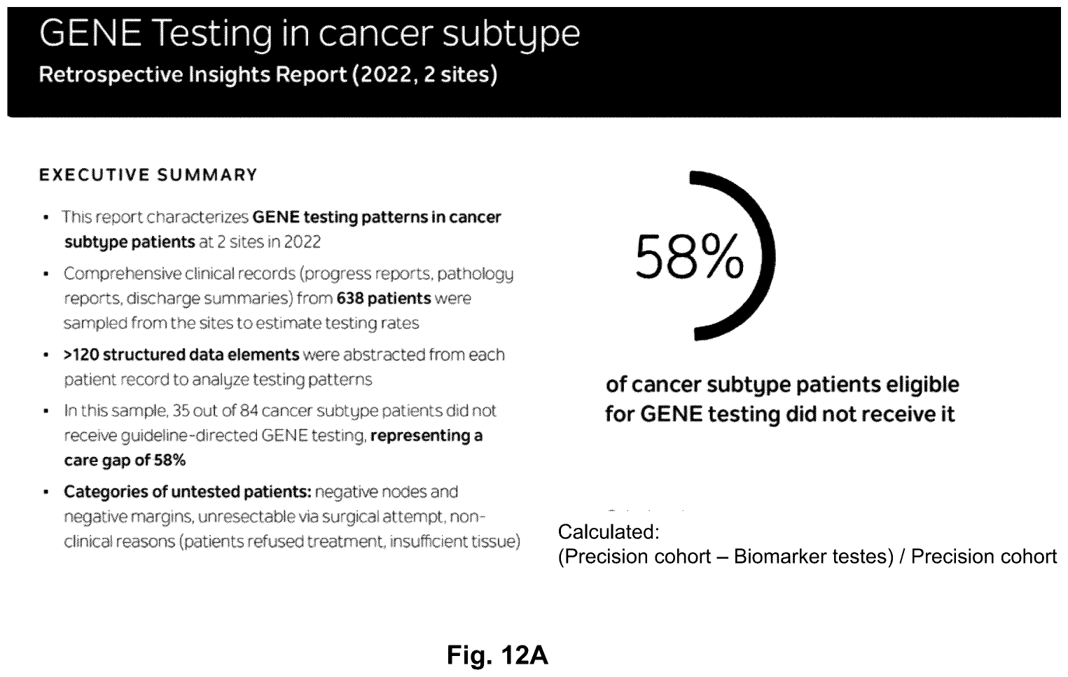

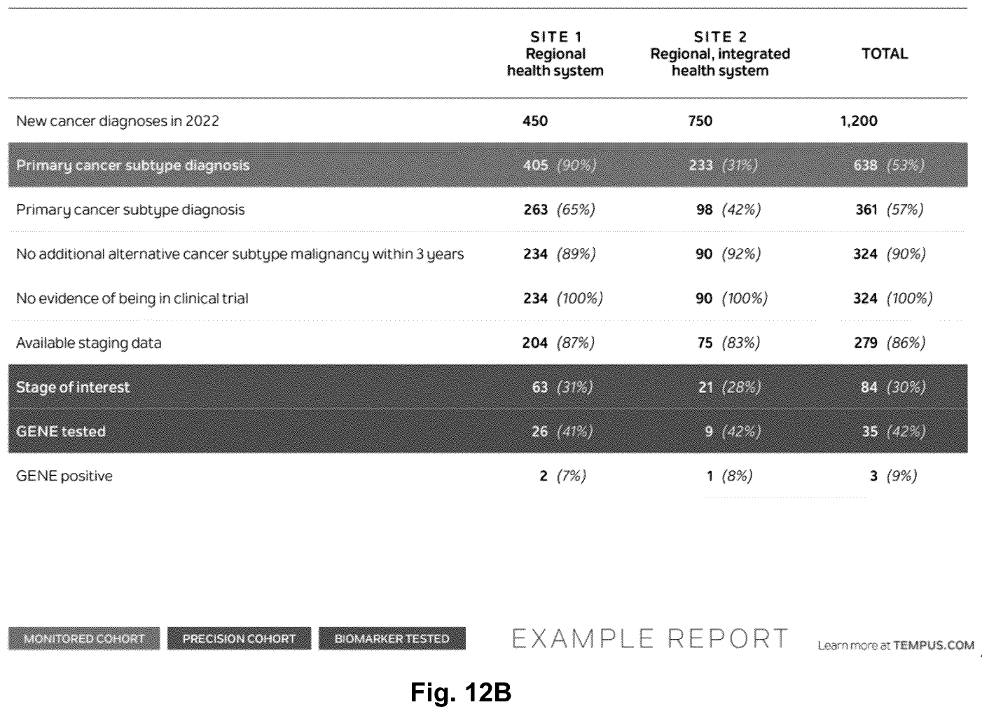

For evaluation of institutional trends, the systems and methods of the present disclosure provides institutional level insight reports to help stakeholders understand effectiveness of the notifications as illustrated in FIGS. 12A and 12B. Emerging trends in guideline adoption and patient outcome interventions like the one Caroline experienced exemplify the transformative potential of artificial intelligence and precision medicine. FIG. 12 illustrates how the present disclosure deliver the technology that saves medical practitioners time.

### IV. Additional Embodiments for Application of Logic to Determine Who Gets Notifications

The following are additional embodiments of the present disclosure directed to determining the path for notifications.

Embodiment 1. A computer system for treatment monitoring, the computer system comprising a memory and a processor, the memory storing instructions executable by the processor to perform a method, the method comprising: obtaining, through a computer network, a plurality of data items associated with the treatment of the subject from multimodal treatment data for a subject; populating a data repository associated with the subject using the plurality of data items; identifying one or more diagnosis associated with the subject, and a plurality of caregivers, using the data repository; accessing a plurality of decision rules, wherein each decision rule in the plurality of decision rules has a corresponding triggering condition and wherein the plurality of decision rules model a plurality of institution based care plans for a plurality of diagnoses including the one or more diagnosis associated with the subject; firing, using an evaluation module, a first decision rule in the plurality of decision rules whose corresponding triggering condition arises in the data repository, wherein the first decision is associated with (i) a first diagnosis in the one or more diagnosis associated with the subject and (ii) a first institution based care plan in the plurality of institution based care plans; identifying a first notification rule in a notification rule repository that is actionable upon the firing of the first decision rule; and communicating a first notification to a first notification path specified by the first notification rule, wherein the first notification path consists of a first subset of the plurality of caregivers thereby monitoring treatment of the subject.

Embodiment 2. The computer system of embodiment 1, wherein the corresponding triggering condition for the first decision rule arises in the data repository when the data repository is missing a diagnostic test result associated with the first diagnosis.

Embodiment 3. The computer system of embodiment 1, wherein the corresponding triggering condition for the first notification rule arises in the data repository when the data repository includes a diagnostic test result that satisfies a diagnostic test result threshold.

Embodiment 4. The computer system of any one of embodiments 1-3, wherein the corresponding triggering condition for the first decision rule is an absence of an evaluation of the subject in the multimodal treatment data for the subject during a predetermined time period for the first diagnosis.

Embodiment 5. The computer system of any one of embodiments 1-4, wherein the corresponding triggering condition for the first decision rule is an indication in the data repository that the subject has been prescribed a combination of medications having a documented adverse pharmacodynamic or pharmacokinetic interaction with each other.

Embodiment 6. The computer system of any one of embodiments 1-5, wherein the corresponding triggering condition for the first decision rule is an absence of an indication in the data repository of a clinical trial for which the subject is currently eligible.

Embodiment 7. The computer system of any one of embodiments 1-6, wherein the corresponding triggering condition for the first decision rule is an absence of a therapy implicated for the first diagnosis in the data repository.

Embodiment 8. The computer system of any one of embodiments 1-7, wherein the corresponding triggering condition for the first decision rule arises in the data repository when the data repository is missing a medical procedure associated with the first diagnosis.

Embodiment 9. The computer system of any one of embodiments 1-8, wherein the corresponding triggering condition for the first decision rule arises in the data repository when the data repository includes a medical procedure result that specifies the subject has a predetermined condition specified by the corresponding triggering condition of the first decision rule.

Embodiment 10. The computer system of any one of embodiments 1-9, wherein the first institution based care plan is at an institution and the first notification path is an identity of a first caregiver having a first specialty associated with treatment of the first diagnosis.

Embodiment 11. The computer system of embodiment 10, the method further comprising: communicating a second notification to a second caregiver having the first specialty associated with treatment of the first diagnosis when the first caregiver fails to respond to the first notification.

Embodiment 12. The computer system of any one of embodiments 1-9, wherein the first institution based care plan is at an institution and the first notification path is an identity of a primary caregiver for the subject at the institution.

Embodiment 13. The computer system of any one of embodiments 1-9, wherein the institution based care plan is at an institution and the first notification path is an identity of a caregiver for the subject that most recently attended to the subject at the institution.

Embodiment 14. The computer system of any one of embodiments 1-9, wherein the first institution based care plan is at an institution and the first notification path is an identity of a caregiver for the subject that most frequently attends to the subject at the institution.

Embodiment 15. The computer system of any one of embodiments 1-9, wherein the first institution based care plan is at an institution and the first notification path is an identity of a caregiver for the subject that has been assigned responsibility for administering an upcoming medical procedure in accordance with the first institution based care plan for the first diagnosis.

Embodiment 16. The computer system of any one of embodiments 1-15, wherein the first notification rule is associated with a first type of caregiver and wherein: when the plurality of caregivers includes a first caregiver of the first type, the first notification path includes the first caregiver of the first type, and when the plurality of caregivers fails to include a first caregiver of the first type, the first notification path includes a primary caregiver associated with the subject.

Embodiment 17. The computer system of any one of embodiments 1-16, the method further comprising: firing, using the evaluation module, a second decision rule in the plurality of decision rules whose corresponding triggering condition arises in the data repository; and suppressing a notification associated with the second decision rule when the first decision rule is fired.

Embodiment 18. The computer system of any one of embodiments 1-16, the method further comprising: firing, using the evaluation module, a second first decision rule in the plurality of decision rules whose corresponding triggering condition arises in the data repository; identifying a second notification rule in the notification rule repository that is actionable upon the firing of the second decision rule, wherein the second notification rule is a hierarchical notification rule that is dependent upon the firing of the first decision rule and the second decision rule; and communicating a second notification to a second notification path specified by the second notification rule, wherein the second notification path consists of a second subset of the plurality of caregivers.

Embodiment 19. The computer system of any one of embodiments 1-18, wherein the plurality of decision rules includes a plurality of subsets of decision rules, where each respective subset of decision rules in the plurality of subsets of decision rules model a respective institution based care plan for a diagnosis in a plurality of diagnoses.

Embodiment 20. The computer system of embodiment 19, the plurality of decision rules model respective institution based care plans for more than 50 diagnoses.

Embodiment 21. The computer system of any one of embodiments 1-20, wherein the plurality of decisions rules is in the form of a directed graph comprising a plurality of nodes and a plurality of edges, each respective node in the plurality of nodes is a decision rule in the plurality of decision rules, and each edge in the plurality of edges links a respective first node in the plurality of nodes with at least one other respective second node in the plurality of nodes and specifies what conditions are to be satisfied by the data in the data repository in order to include the decision rule of the at least one other respective second node in the first subset of decision rules when the decision rule of the first respective node is included in the first subset of decision rules.

Embodiment 22. The computer system of any one of embodiments 1-20, wherein the plurality of decisions rules is in the form of a decision tree comprising a plurality of decisions, and each respective decision in the plurality of decisions dictates whether or not to include a decision rule in the plurality of decision rules in the first subset of decision rules based on satisfying or failing to satisfy one or more conditions using data in the data repository.

### V. Additional Embodiments for Selection of Decision Rules for Condition

The following are additional embodiments directed to selection of decision rules for a condition,

Embodiment 23. A computer system for treatment monitoring, the computer system comprising a memory and a processor, the memory storing instructions executable by the processor to perform a method, the method comprising: obtaining, through a computer network, a plurality of data items associated with the treatment of the subject from multimodal treatment data for a subject; populating a data repository associated with the subject using the plurality of data items; identifying one or more diagnosis associated with the subject, and a plurality of caregivers, using the data repository; selecting a first subset of decision rules from among a plurality of decision rules based on a first diagnosis in the one or more diagnoses, wherein each decision rule in the first subset of decision rules has a corresponding triggering condition and wherein the subset of decision rules model a first institution based care plan for the first diagnosis; firing, using an evaluation module, a first decision rule in the first subset of decision rules whose corresponding triggering condition arises in the data repository wherein the firing generates a first notification, through the computer network, that the first decision rule has been fired, thereby monitoring treatment of the subject.

Embodiment 24. The computer system of embodiment 23, wherein the corresponding triggering condition for the first decision rule arises in the data repository when the data repository is missing a diagnostic test result associated with the first diagnosis.

Embodiment 25. The computer system of embodiment 23 or 24, wherein the corresponding triggering condition for the first decision rule arises in the data repository when the data repository includes a diagnostic test result that satisfies a diagnostic test result threshold.

Embodiment 26. The computer system of any one of embodiments 23-25, wherein the corresponding triggering condition for the first decision rule comprises an absence of an evaluation of the subject in the multimodal treatment data for the subject during a predetermined time period for the first diagnosis.

Embodiment 27. The computer system of any one of embodiments 23-26, wherein the corresponding triggering condition for the first decision rule comprises an indication in the data repository that the subject has been prescribed a combination of medications having a documented adverse pharmacodynamic or pharmacokinetic interaction with each other.

Embodiment 28. The computer system of any one of embodiments 23-27, wherein the corresponding triggering condition for the first decision rule comprises an absence of an indication in the data repository of a clinical trial for which the subject is currently eligible.

Embodiment 29. The computer system of any one of embodiments 23-28, wherein the corresponding triggering decision for the first notification rule comprises an absence of a therapy implicated for the first diagnosis in the data repository.

Embodiment 30. The computer system of any one of embodiments 23-29, wherein the corresponding triggering condition for the first decision rule arises in the data repository when the data repository is missing a medical procedure associated with the first diagnosis.

Embodiment 31. The computer system of any one of embodiments 23-30, wherein the corresponding triggering condition for the first decision rule arises in the data repository when the data repository includes a medical procedure result that specifies the subject has a predetermined condition specified by the corresponding triggering condition of the first decision rule.

Embodiment 32. The computer system of any one of embodiments 23-31, wherein the first institution based care plan is at an institution and the first notification is sent to a first caregiver having a first specialty associated with treatment of the first diagnosis.

Embodiment 33. The computer system of embodiment 32, the method further comprising: communicating a second notification to a second caregiver having the first specialty associated with treatment of the first diagnosis when the first caregiver fails to respond to the first notification.

Embodiment 34. The computer system of any one of embodiments 23-31, wherein the first institution based care plan is at an institution and the first notification is sent to a primary caregiver for the subject at the institution.

Embodiment 35. The computer system of any one of embodiments 23-31, wherein the institution based care plan is at an institution and the first notification is sent to caregiver for the subject that most recently attended to the subject at the institution.

Embodiment 36. The computer system of any one of embodiments 13-31, wherein the first institution based care plan is at an institution and the first notification is sent to a caregiver for the subject that most frequently attends to the subject at the institution.

Embodiment 37. The computer system of any one of embodiments 23-31, wherein the first institution based care plan is at an institution and the first notification is sent to a caregiver for the subject that has been assigned responsibility for administering an upcoming medical procedure in accordance with the first institution based care plan for the first diagnosis.

Embodiment 38. The computer system of any one of embodiments 23-37, wherein the first notification rule is associated with a first type of caregiver and wherein: when the plurality of caregivers includes a first caregiver of the first type, the first notification is sent to the first caregiver of the first type, and when the plurality of caregivers fails to include a first caregiver of the first type, the first notification is sent to a primary caregiver associated with the subject.

Embodiment 39. The computer system of any one of embodiments 23-38, the method further comprising: firing, using the evaluation module, a second decision rule in the first subset of decision rules whose corresponding triggering condition arises in the data repository; and suppressing a notification associated with the second decision rule when the first decision rule is fired.

Embodiment 40. The computer system of any one of embodiments 23-39, wherein the plurality of decision rules includes a plurality of subsets of decision rules, where each respective subset of decision rules in the plurality of subsets of decision rules model a respective institution based care plan for a diagnosis in a plurality of diagnoses.

Embodiment 41. The computer system of embodiment 40, the plurality of decision rules model respective institution based care plans for more than 50 diagnoses.

Embodiment 42. The computer system of any one of embodiments 23-41, wherein the first subset of decision rules models the first institution based care plan for the first diagnosis as a first decision tree.

Embodiment 43. The computer system of any one of embodiments 23-41, wherein the plurality of decisions rules is in the form of a directed graph comprising a plurality of nodes and a plurality of edges, each respective node in the plurality of nodes is a decision rule in the plurality of decision rules, each edge in the plurality of edges links a respective first node in the plurality of nodes with at least one other respective second node in the plurality of nodes and specifies what conditions are to be satisfied by the data in the data repository in order to include the decision rule of the at least one other respective second node in the first subset of decision rules when the decision rule of the first respective node is included in the first subset of decision rules, and the selecting the first subset of decision rules from among the plurality of decision rules based on the first diagnosis comprises traversing the directed graph beginning at an initial node in the directed graph.

Embodiment 44. The computer system of any one of embodiments 23-41, wherein the plurality of decisions rules is in the form of a decision tree comprising a plurality of decisions, each respective decision in the plurality of decisions dictates whether or not to include a decision rule in the plurality of decision rules in the first subset of decision rules based on satisfying or failing to satisfy one or more conditions using data in the data repository, and the selecting the first subset of decision rules from among the plurality of decision rules based on the first diagnosis comprises traversing the decision tree beginning at an initial decision in the decision tree.

### VI. Additional Embodiments Computer Systems and Non-Transitory Computer-Readable Storage Media

Another aspect of the present disclosure provides a computer system comprising one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

Although inventions have been particularly shown and described with reference to a preferred embodiment and various alternate embodiments, it will be understood by persons skilled in the relevant art that various changes in form and details can be made therein without departing from the spirit and scope of the invention.

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### Additional Considerations

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter, in some embodiments, includes not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein, in some embodiments, are claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

1. A computer system for check point identification for a subject through in silico modeling, the computer system comprising a memory and a processor, the memory storing a plurality of processor executable instructions executable by the processor, the plurality of processor executable instructions comprising:
instructions for identifying one or more first conditions associated with a subject, and a plurality of contacts associated with the subject, in a data repository comprising a plurality of treatment information units associated with the subject;
instructions for discovering a first subset of decision rules from among a plurality of decision rules through a modeling procedure that aligns a first condition in the one or more first conditions against the plurality of decision rules, wherein
each decision rule in the first subset of decision rules has a corresponding triggering condition, and
the first subset of decision rules models a response to the first condition at a first entity;
instructions for activating, using an evaluation module, a first decision rule in the first subset of decision rules whose corresponding triggering condition arises in the data repository;
instructions for identifying a first notification rule in a notification rule repository that is actionable upon the activating of the first decision rule; and
instructions for communicating a first notification to a first notification path in accordance with the first notification rule, using a computer network, wherein the first notification path consists of a first subset of the plurality of contacts.

2. The computer system of claim 1, wherein the plurality of instructions further comprises instructions for updating the data repository using multimodal treatment data for the subject.

3. The computer system of claim 1, wherein the one or more first conditions comprises a first diagnosis associated with the subject.

4. The computer system of claim 1, wherein the plurality of contacts associated with the subject comprises:
a third party sponsoring a clinical trial, or
a plurality of caregivers that treat the subject at a first institution.

5. The computer system of claim 1, wherein the plurality of contacts associated with the subject includes the subject.

6. (canceled)

7. The computer system of claim 1, wherein the corresponding triggering condition for the first decision rule arises in the data repository when:
the data repository is missing a diagnostic test result specified by the first decision rule, or
the data repository includes a diagnostic test result that satisfies a diagnostic test result threshold.

8. (canceled)

9. The computer system of claim 1, wherein
the plurality of instructions further comprises instructions for updating the data repository using multimodal treatment data for the subject, and
the corresponding triggering condition for the first decision rule comprises an absence of an evaluation of the subject in the multimodal treatment data for the subject during a predetermined time period associated with the first decision rule.

10. The computer system of claim 1, wherein the corresponding triggering condition for the first decision rule comprises:
an indication in the data repository that the subject has been prescribed a combination of medications having a documented adverse pharmacodynamic or pharmacokinetic interaction with each other,
an absence of an indication in the data repository of a clinical trial for which the subject is currently eligible, or
an absence of a therapy in the data repository.

11. (canceled)

12. (canceled)

13. The computer system of claim 1, wherein the corresponding triggering condition for the first decision rule arises in the data repository when:
the data repository is missing a medical procedure, or
the data repository includes a medical procedure result that specifies the subject has a predetermined condition specified by the corresponding triggering condition of the first decision rule.

14. (canceled)

15. The computer system of claim 1, wherein the response to the first condition at the first entity is a first institution based care plan at an institution and the first notification path is an identity of a first contact in the plurality of contacts having a first specialty associated with activation of the first decision rule.

16. The computer system of claim 15, the plurality of instructions further comprising:
communicating a second notification to a second contact having the first specialty when the first contact fails to respond to the first notification.

17. The computer system of claim 1,
wherein the response to the first condition at the first entity is a first institution based care plan at an institution and the first notification path is an identity of a primary caregiver for the subject, in the plurality of contacts associated with the subject, at the institution, or,
wherein the response to the first condition at the first entity is a first institution based care plan at an institution and the first notification path is an identity of a caregiver for the subject, in the plurality of contacts associated with the subject, that most recently attended to the subject at the institution,
wherein the response to the first condition at the first entity is a first institution based care plan at an institution and the first notification path is an identity of a caregiver for the subject, in the plurality of contacts associated with the subject, that most frequently attends to the subject at the institution.

18. (canceled)

19. (canceled)

20. The computer system of claim 1, wherein the response to the first condition at the first entity is a first institution based care plan at an institution and the first notification path is an identity of a caregiver for the subject, in the plurality of contacts associated with the subject, that has been assigned responsibility for administering an upcoming medical procedure in accordance with the first institution based care plan.

21. The computer system of claim 1, wherein the first notification rule is associated with a first type of caregiver and wherein:
when the plurality of contacts associated with the subject includes a first caregiver of the first type, the first notification path includes the first caregiver of the first type, and
when the plurality of contacts associated with the subject does not include a first caregiver of the first type, the first notification path includes a primary caregiver, in the plurality of contacts associated with the subject, associated with the subject.

22. The computer system of claim 1, wherein the plurality of processor executable instructions further comprises:
instructions for firing, using the evaluation module, a second decision rule in the first subset of decision rules whose corresponding triggering condition arises in the data repository; and
suppressing a notification associated with the second decision rule when the first decision rule is fired.

23. The computer system of claim 1, wherein the plurality of processor executable instructions further comprises:
instructions for firing, using the evaluation module, a second decision rule in the first subset of decision rules whose corresponding triggering condition arises in the data repository;
identifying a second notification rule in the notification rule repository that is actionable upon the firing of the second decision rule, wherein the second notification rule is a hierarchical notification rule that is dependent upon the firing of the first decision rule; and
communicating a second notification to a second notification path specified by the second notification rule, wherein the second notification path consists of a second subset of the plurality of contacts.

24. The computer system of claim 1,
wherein the plurality of decision rules includes a plurality of subsets of decision rules, wherein each respective subset of decision rules in the plurality of subsets of decision rules models a respective institution based care plan for a diagnosis in a plurality of diagnoses.

25. (canceled)

26. The computer system of claim 1, wherein the response to the first condition at the first entity is a first institution based care plan at an institution and wherein the first subset of decision rules model the first institution based care plan for a first diagnosis indicated by the activating of the first decision rule as a first decision tree.

27. The computer system of claim 1, wherein
the plurality of decisions rules is in the form of a directed graph comprising a plurality of nodes and a plurality of edges,
each respective node in the plurality of nodes is a decision rule in the plurality of decision rules,
each edge in the plurality of edges links a respective first node in the plurality of nodes with at least one other respective second node in the plurality of nodes and specifies what conditions are to be satisfied by the data in the data repository in order to include the decision rule of the at least one other respective second node in the first subset of decision rules when the decision rule of the first respective node is included in the first subset of decision rules, and
the instructions for discovering the first subset of decision rules from among the plurality of decision rules through a modeling procedure that aligns a first condition in the one or more first conditions against the plurality of decision rules comprises traversing the directed graph beginning at an initial node in the directed graph.

28. The computer system of claim 1, wherein
the plurality of decisions rules is in the form of a decision tree comprising a plurality of decisions,
each respective decision in the plurality of decisions dictates whether or not to include a decision rule in the plurality of decision rules in the first subset of decision rules based on satisfying or failing to satisfy one or more conditions using data in the data repository, and
the instruction for discovering the first subset of decision rules from among the plurality of decision rules through a modeling procedure that aligns a first condition in the one or more first conditions against the plurality of decision rules comprises traversing the decision tree beginning at an initial decision in the decision tree.
